(19) United States
(12) Patent Application Publication
(10) Pub. No.: US 2022/0002726 A1
(43) Pub. Date: Jan. 6, 2022

(54) METHODS OF SAFE ADMINISTRATION OF AN IRF5 ANTISENSE OLIGONUCLEOTIDE

(71) Applicant: Janssen Biotech, Inc., Horsham, PA (US)

(72) Inventors: Damayanthi Devineni, Belle Mead, NJ (US); Bart Frederick, Downingtown, PA (US); Cathye Shu, Lansdale, PA (US)

(21) Appl. No.: 17/364,396

(22) Filed: Jun. 30, 2021

Related U.S. Application Data
(60) Provisional application No. 63/047,695, filed on Jul. 2, 2020.
(60) Provisional application No. 63/047,169, filed on Jul. 1, 2020.

(51) Int. Cl. C12N 15/113

(52) U.S. Cl. CPC C12N 15/113; C12N 2320/30; C12N 2310/3341; C12N 2310/322; C12N 2310/341; C12N 2310/11; C12N 2310/321; C12N 2310/315

(57) ABSTRACT

Methods of treating a human subject having a disease associated with Interferon Regulatory Factor 5 (IRF5) comprise administering to the subject an antisense oligomer to an IRF 5 nucleic acid.

### CROSS-REFERENCE TO RELATED APPLICATIONS

This application claims priority to and the benefit of U.S. Provisional Patent Application No. 63/047,169, filed Jul. 1, 2020; and U.S. Provisional Patent Application No. 63/047,695, filed Jul. 2, 2020; the disclosures of each of which are hereby incorporated herein by reference in their entireties.

### SUBMISSION OF SEQUENCE LISTING ON ASCII TEXT FILE

The content of the following submission on ASCII text file is incorporated herein by reference in its entirety; a computer readable form (CRF) of the Sequence Listing (JBI6348USNP1_SL.txt, created on Jun. 30, 2021, and is 64,959 bytes in size.).

### FIELD OF THE INVENTION

The present invention relates to methods and compositions for inhibiting the expression and/or reducing the amount of Interferon Regulatory Factor 5 in a human subject and, in particular, to the treatment, prevention and/or amelioration of a disease associated with Interferon Regulatory Factor 5.

### BACKGROUND

Inflammatory bowel diseases (IBD) are characterized by intestinal inflammation, disruption of the epithelial barrier, and microbial dysbiosis. The cause of IBD remains unknown, but evidence suggests that IBD results from an inappropriate inflammatory response to intestinal microbes in a genetically susceptible host. The two major forms of inflammatory bowel disease in humans are Crohn's disease and ulcerative colitis (UC). The current standard of medical care involves treatment with anti-inflammatory agents, corticosteroids, immunomodulators, including azathioprine, or its active metabolite 6-mercaptopurine, methotrexate, biologic agents, including tumor necrosis factor antagonist therapies, anti-integrin therapies, and anti-interleukin (IL) 12/23 therapy. However, many patients do not fully respond to these therapeutic approaches and such treatments may lose efficacy over time.

Genome-wide association studies (GWAS) have linked the risk of IBD to single nucleotide polymorphisms (SNPs) in over 200 loci. A number of these IBD risk polymorphisms are associated with genes involved in macrophage and dendritic cell function, including Interferon Regulatory Factor 5 (IRF5) which is a transcription factor that regulates inflammatory and immune responses. The IBD risk polymorphisms in the IRF5 gene are associated with elevated expression of IRF5. Eames, H. L., et al., Interferon regulatory factor 5 in human autoimmunity and murine models of autoimmune disease. Transl Res. 167(1):167-182 (2016): Graham, R. R., et al., Three functional variants of IFN regulatory factor 5 (IRF5) define risk and protective haplotypes for human lupus, Proc. Natl. Acad. Sci. 104(16):6758-6763 (2007); Hedl, M., et al., IRF5 risk polymorphisms contribute to interindividual variance in pattern recognition receptor-mediated cytokine secretion in human monocyte-derived cells, J. Immunol. 188(11):5348-5356 (2012); Hedl, M., et al., IRF5 and IRF5 Disease-Risk Variants Increase Glycolysis and Human M1 Macrophage Polarization by Regulating Proximal Signaling and Akt2 Activation, Cell Rep. 16(9):2442-2455 (2016).

Studies of IRF5 indicate its involvement in macrophage and dendritic cell activation in response to bacterial stimuli and subsequent cytokine expression. IRF5 knockout mice are viable and resistant to endotoxic shock, highlighting a critical role of IRF5 in response to Toll-like receptor (TLR) 4 signaling. IRF5 heterozygous mice demonstrate an intermediate release of cytokines in response to multiple TLR stimulations as compared to wild type or a homozygous deletion. In vivo studies have shown that small interfering ribonucleic acids (siRNA) targeting IRF5 are able to lower production of the proinflammatory cytokines in mice, including TNFa and interleukins IL-12, IL-23 and IL-1β, as well as reduce the number of proinflammatory macrophages.

The effects of knocking down IRF5 have not been fully investigated in human disease, but studies suggest that lowering levels of IRF5 expression is a promising therapeutic strategy for IBD. Macrophages isolated from donors with increased IRF5 expression presented an increased release of proinflammatory cytokines after bacterially derived muramyl dipeptide stimulation. Monocyte-derived cells from donors carrying IRF5 risk alleles show increased secretion of pattern recognition receptor-induced cytokines. Gain of function SNPs in IRF5 have been identified in a number of autoimmune diseases including IBD, systemic lupus erythematosus, and rheumatoid arthritis. In addition, IRF5 siRNA was effective in reducing TNFa, IL-12/23 and IL-1β in human inflammatory macrophages in vitro.

Therefore, it would be desirable to provide compositions and methods for targeting IRF5 expression that have high efficacy and tolerability in human subjects in the treatment of IBD and other diseases associated with IRF5.

### BRIEF SUMMARY OF THE INVENTION

Methods of treating a human subject having a disease associated with Interferon Regulatory Factor 5 (IRF5) are described, that comprise administering to the subject a safe and effective amount of an antisense oligomer to an IRF 5 nucleic acid. In one embodiment, the antisense oligomer is administered in an amount of about 120 mg/day to about 1200 mg/day, such as a once daily oral administration of 120 mg/day, 360 mg/day, 720 mg/day or 1200 mg/day.

In one embodiment the antisense oligomer comprises an oligonucleotide that is complementary to an IRF5 nucleic acid. In certain embodiments, the oligonucleotide has a nucleobase sequence that is complementary to an IRF5 nucleic acid selected from the group consisting of: SEQ ID NO: 10, SEQ ID NO: 11, SEQ ID NO: 12, SEQ ID NO: 13, SEQ ID NO: 14, SEQ ID NO: 15, and SEQ ID NO: 16. In certain embodiments, the oligonucleotide is a modified oligonucleotide that comprises at least one modification selected from the group consisting of: a modified sugar, a modified internucleoside linkage, a modified nucleobase, and combinations thereof. In certain such embodiments, the modified oligonucleotide comprises a gapmer motif having a 5′ wing segment consisting of three linked nucleosides, a 3′ wing segment consisting of three linked nucleosides, and a gap segment consisting of ten linked deoxynucleosides that is positioned between the 5′ wing segment and the 3′ wing segment, and wherein each nucleoside of the wing segments comprises a cEt sugar, each internucleoside linkage is a phosphorothioate linkage, and each cytosine is a 5-methylcytosine.

### BRIEF DESCRIPTION OF THE DRAWINGS

The foregoing summary and the following detailed description of the invention will be better understood when read in conjunction with the appended drawings. For the purpose of illustrating the invention, there are shown in the drawings embodiments which are presently preferred. It should be understood, however, that the invention is not limited to the precise embodiments shown in the drawings.

FIG. 1 is a schematic diagram showing an overview of a study for evaluating the single and multiple dose safety, tolerability, pharmacokinetics, pharmacodynamics, and immunogenicity of an antisense oligomer targeted to an IRF5 nucleic acid.

### DETAILED DESCRIPTION OF THE INVENTION

Methods are described for preventing, ameliorating, or otherwise treating diseases in an individual that are associated with Interferon Regulatory Factor 5 (IRF5). Examples of such diseases include inflammatory bowel disease (IBD), ulcerative colitis, Crohn's disease, systemic lupus erythematosus (SLE), rheumatoid arthritis, primary biliary cirrhosis, systemic sclerosis, Sjogren's syndrome, multiple sclerosis, scleroderma, interstitial lung disease (SSc-ILD), polycystic kidney disease (PKD), chronic kidney disease (CKD), nonalcoholic steatohepatitis (NASH), liver fibrosis, asthma, and severe asthma.

In one embodiment, the method of treating a disease associated with IRF5 comprises administering a compound to an individual that targets IRF5 to inhibit IRF5 expression, and preferably comprises administering to the individual a compound comprising an IRF5 specific inhibitor. In further embodiments, the individual is identified as having, or at risk of having an inflammatory disease, such as an inflammatory gastrointestinal disease. In a preferred embodiment, the gastrointestinal disease is ulcerative colitis or Crohn's disease.

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the present invention. Such discussion is not an admission that any or all of these matters form part of the prior art with respect to any inventions disclosed or claimed.

It is to be understood that both the foregoing general description and the following detailed description are exemplary and explanatory only and are not restrictive of the embodiments, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting.

The section headings used herein are for organizational purposes only and are not to be construed as limiting the subject matter described. All documents, or portions of documents, cited in this application, including, but not limited to, patents, patent applications, articles, books, treatises, and GenBank and NCBI reference sequence records are hereby expressly incorporated by reference for the portions of the document discussed herein, as well as in their entirety.

It is understood that the sequence set forth in each SEQ ID NO in the examples contained herein is independent of any modification to a sugar moiety, an internucleoside linkage, or a nucleobase. As such, compounds defined by a SEQ ID NO may comprise, independently, one or more modifications to a sugar moiety, an internucleoside linkage, or a nucleobase. Compounds described by ION number indicate a combination of nucleobase sequence, chemical modification, and motif.

### Definitions

Unless otherwise indicated, the following terms have the following meanings:

“2′-deoxyfuranosyl sugar moiety” or “2′-deoxyfuranosyl sugar” means a furanosyl sugar moiety having two hydrogens at the 2′-position. 2′-deoxyfuranosyl sugar moieties may be unmodified or modified and may be substituted at positions other than the 2′-position or unsubstituted. A β-D-2′-deoxyribosyl sugar moiety in the context of an oligonucleotide is an unsubstituted, unmodified 2′-deoxyfuranosyl and is found in naturally occurring deoxyribonucleic acids (DNA).

“2′-deoxynucleoside” means a nucleoside comprising 2′-H(H) furanosyl sugar moiety, as found in naturally occurring deoxyribonucleic acids (DNA). In certain embodiments, a 2′-deoxynucleoside may comprise a modified nucleobase or may comprise an RNA nucleobase (uracil).

“2′-O-methoxyethyl” (also 2′-MOE) refers to a 2′-O(CH₂)₂—OCH₃ in the place of the 2′-OH group of a ribosyl ring. A 2′-O-methoxyethyl modified sugar is a modified sugar. “2′-MOE nucleoside” (also 2′-O-methoxyethyl nucleoside) means a nucleoside comprising a 2′-MOE modified sugar moiety.

“2′-substituted nucleoside” or “2-modified nucleoside” means a nucleoside comprising a 2′-substituted or 2′-modified sugar moiety. As used herein, “2′-substituted” or “2-modified” in reference to a sugar moiety means a sugar moiety comprising at least one 2′-substituent group other than H or OH.

“3′ target site” refers to the nucleotide of a target nucleic acid which is complementary to the 3′-most nucleotide of a particular compound.

“5′ target site” refers to the nucleotide of a target nucleic acid which is complementary to the 5′-most nucleotide of a particular compound. “5-methylcytosine” means a cytosine with a methyl group attached to the 5 position. “About” means within +10°,% of a value. For example, if it is stated, “the compounds affected about 70% inhibition of PNPLA3”, it is implied that PNPLA3 levels are inhibited within a range of 60% and 80%.

“Administration” or “administering” refers to routes of introducing a compound or composition provided herein to an individual to perform its intended function. An example of a route of administration that can be used includes, but is not limited to parenteral administration, such as subcutaneous, intravenous, or intramuscular injection or infusion.

“Administered concomitantly” or “co-administration” means administration of two or more compounds in any manner in which the pharmacological effects of both are manifest in the patient. Concomitant administration does not require that both compounds be administered in a single pharmaceutical composition, in the same dosage form, by the same route of administration, or at the same time. The effects of both compounds need not manifest themselves at the same time. The effects need only be overlapping for a period of time and need not be coextensive. Concomitant administration or co-administration encompasses administration in parallel or sequentially.

“Amelioration” refers to an improvement or lessening of at least one indicator, sign, or symptom of an associated disease, disorder, or condition. In certain embodiments, amelioration includes a delay or slowing in the progression or severity of one or more indicators of a condition or disease. The progression or severity of indicators may be determined by subjective or objective measures, which are known to those skilled in the art.

“Animal” refers to a human or non-human animal, including, but not limited to, mice, rats, rabbits, dogs, cats, pigs, and non-human primates, including, but not limited to, monkeys and chimpanzees.

“Antisense activity” means any detectable and/or measurable activity attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound to the target.

“Antisense compound” means a compound comprising an oligonucleotide and optionally one or more additional features, such as a conjugate group or terminal group. Examples of antisense compounds include single-stranded and double-stranded compounds, such as, oligonucleotides, ribozymes, siRNAs, shRNAs, ssRNAs, and occupancy-based compounds.

“Antisense inhibition” means reduction of target nucleic acid levels in the presence of an antisense compound complementary to a target nucleic acid compared to target nucleic acid levels in the absence of the antisense compound.

“Antisense mechanisms” are all those mechanisms involving hybridization of a compound with target nucleic acid, wherein the outcome or effect of the hybridization is either target degradation or target occupancy with concomitant stalling of the cellular machinery involving, for example, transcription or splicing.

“Antisense oligonucleotide” means an oligonucleotide having a nucleobase sequence that is complementary to a target nucleic acid or region or segment thereof. In certain embodiments, an antisense oligonucleotide is specifically hybridizable to a target nucleic acid or region or segment thereof.

“Bicyclic nucleoside” or “BNA” means a nucleoside comprising a bicyclic sugar moiety. “Bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety.

“Branching group” means a group of atoms having at least 3 positions that are capable of forming covalent linkages to at least 3 groups. In certain embodiments, a branching group provides a plurality of reactive sites for connecting tethered ligands to an oligonucleotide via a conjugate linker and/or a cleavable moiety.

“Cell-targeting moiety” means a conjugate group or portion of a conjugate group that is capable of binding to a particular cell type or particular cell types.

“cEt” or “constrained ethyl” means a ribosyl bicyclic sugar moiety wherein the second ring of the bicyclic sugar is formed via a bridge connecting the 4′-carbon and the 2′-carbon, wherein the bridge has the formula: 4′-CH(CH₃)—O-2′, and wherein the methyl group of the bridge is in the S configuration.

“cEt nucleoside” means a nucleoside comprising a cEt modified sugar moiety. “Chemical modification” in a compound describes the substitutions or changes through chemical reaction, of any of the units in the compound relative to the original state of such unit. “Modified nucleoside” means a nucleoside having, independently, a modified sugar moiety and/or modified nucleobase. “Modified oligonucleotide” means an oligonucleotide comprising at least one modified internucleoside linkage, a modified sugar, and/or a modified nucleobase.

“Chemically distinct region” refers to a region of a compound that is in some way chemically different than another region of the same compound. For example, a region having 2′-O-methoxyethyl nucleotides is chemically distinct from a region having nucleotides without 2′-O-methoxyethyl modifications.

“Chimeric antisense compounds” means antisense compounds that have at least 2 chemically distinct regions, each position having a plurality of subunits.

“Cleavable bond” means any chemical bond capable of being split. In certain embodiments, a cleavable bond is selected from among: an amide, a polyamide, an ester, an ether, one or both esters of a phosphodiester, a phosphate ester, a carbamate, a di-sulfide, or a peptide.

“Cleavable moiety” means a bond or group of atoms that is cleaved under physiological conditions, for example, inside a cell, an animal, or a human.

“Complementary” in reference to an oligonucleotide means the nucleobase sequence of such oligonucleotide or one or more regions thereof matches the nucleobase sequence of another oligonucleotide or nucleic acid or one or more regions thereof when the two nucleobase sequences are aligned in opposing directions. Nucleobase matches or complementary nucleobases, as described herein, are limited to the following pairs: adenine (A) and thymine (T), adenine (A) and uracil (U), cytosine (C) and guanine (G), and 5-methyl cytosine (mC) and guanine (G) unless otherwise specified. Complementary oligonucleotides and/or nucleic acids need not have nucleobase complementarity at each nucleoside and may include one or more nucleobase mismatches. By contrast, “fully complementary” or “100% complementary” in reference to oligonucleotides means that such oligonucleotides have nucleobase matches at each nucleoside without any nucleobase mismatches.

“Conjugate group” means a group of atoms that is attached to an oligonucleotide. Conjugate groups include a conjugate moiety and a conjugate linker that attaches the conjugate moiety to the oligonucleotide.

“Conjugate linker” means a group of atoms comprising at least one bond that connects a conjugate moiety to an oligonucleotide.

“Conjugate moiety” means a group of atoms that is attached to an oligonucleotide via a conjugate linker.

“Contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

“Designing” or “Designed to” refer to the process of designing a compound that specifically hybridizes with a selected nucleic acid molecule.

“Diluent” means an ingredient in a composition that lacks pharmacological activity, but is pharmaceutically necessary or desirable. For example, the diluent in an injected composition can be a liquid, e.g. saline solution.

“Differently modified” means chemical modifications or chemical substituents that are different from one another, including absence of modifications. Thus, for example, a MOE nucleoside and an unmodified DNA nucleoside are “differently modified,” even though the DNA nucleoside is unmodified. Likewise, DNA and RNA are “differently modified,” even though both are naturally-occurring unmodified nucleosides. Nucleosides that are the same but for comprising different nucleobases are not differently modified. For example, a nucleoside comprising a 2′-OMe modified sugar and an unmodified adenine nucleobase and a nucleoside comprising a 2′-OMe modified sugar and an unmodified thymine nucleobase are not differently modified.

“Dose” means a specified quantity of a compound or pharmaceutical agent provided in a single administration, or in a specified time period. In certain embodiments, a dose may be administered in two or more boluses, tablets, or injections. For example, in certain embodiments, where subcutaneous administration is desired, the desired dose may require a volume not easily accommodated by a single injection. In such embodiments, two or more injections may be used to achieve the desired dose. In certain embodiments, a dose may be administered in two or more injections to minimize injection site reaction in an individual. In other embodiments, the compound or pharmaceutical agent is administered by infusion over an extended period of time or continuously. Doses may be stated as the amount of pharmaceutical agent per hour, day, week or month.

“Dosing regimen” is a combination of doses designed to achieve one or more desired effects.

“Double-stranded antisense compound” means an antisense compound comprising two oligomeric compounds that are complementary to each other and form a duplex, and wherein one of the two said oligomeric compounds comprises an oligonucleotide.

“Effective amount” means the amount of compound sufficient to effectuate a desired physiological outcome in an individual in need of the compound. The effective amount may vary among individuals depending on the health and physical condition of the individual to be treated, the taxonomic group of the individuals to be treated, the formulation of the composition, assessment of the individual's medical condition, and other relevant factors.

“Efficacy” means the ability to produce a desired effect.

“Expression” includes all the functions by which a gene's coded information is converted into structures present and operating in a cell. Such structures include, but are not limited to, the products of transcription and translation.

“Gapmer” means an oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.”

“Hybridization” means the annealing of oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases. In certain embodiments, complementary nucleic acid molecules include, but are not limited to, an antisense compound and a nucleic acid target. In certain embodiments, complementary nucleic acid molecules include, but are not limited to, an oligonucleotide and a nucleic acid target.

“Immediately adjacent” means there are no intervening elements between the immediately adjacent elements of the same kind (e.g. no intervening nucleobases between the immediately adjacent nucleobases).

“Individual” means a human or non-human animal selected for treatment or therapy. “Inhibiting the expression or activity” refers to a reduction or blockade of the expression or activity relative to the expression of activity in an untreated or control sample and does not necessarily indicate a total elimination of expression or activity.

“Internucleoside linkage” means a group or bond that forms a covalent linkage between adjacent nucleosides in an oligonucleotide. “Modified internucleoside linkage” means any internucleoside linkage other than a naturally occurring, phosphate internucleoside linkage. Non-phosphate linkages are referred to herein as modified internucleoside linkages.

“IRF5” means any nucleic acid or protein of IRF5. “IRF5 nucleic acid” means any nucleic acid encoding IRF5. For example, in certain embodiments, an IRF5 nucleic acid includes a DNA sequence encoding IRF5, an RNA sequence transcribed from DNA encoding IRF5 (including genomic DNA comprising introns and exons), and an mRNA sequence encoding IRF5. “IRF5 mRNA” means an mRNA encoding a IRF5 protein. The target may be referred to in either upper or lower case.

“IRF5 specific inhibitor” refers to any agent capable of specifically inhibiting IRF5 RNA and/or IRF5 protein expression or activity at the molecular level. For example, IRF5 specific inhibitors include nucleic acids (including antisense compounds), peptides, antibodies, small molecules, and other agents capable of inhibiting the expression of IRF5 RNA and/or IRF5 protein.

“Lengthened oligonucleotides” are those that have one or more additional nucleosides relative to an oligonucleotide disclosed herein, e.g. a parent oligonucleotide.

“Linked nucleosides” means adjacent nucleosides linked together by an internucleoside linkage.

“Linker-nucleoside” means a nucleoside that links an oligonucleotide to a conjugate moiety. Linker-nucleosides are located within the conjugate linker of a compound. Linker-nucleosides are not considered part of the oligonucleotide portion of a compound even if they are contiguous with the oligonucleotide.

“Mismatch” or “non-complementary” means a nucleobase of a first oligonucleotide that is not complementary to the corresponding nucleobase of a second oligonucleotide or target nucleic acid when the first and second oligonucleotides are aligned. For example, nucleobases including but not limited to a universal nucleobase, inosine, and hypoxanthine, are capable of hybridizing with at least one nucleobase but are still mismatched or non-complementary with respect to nucleobase to which it hybridized. As another example, a nucleobase of a first oligonucleotide that is not capable of hybridizing to the corresponding nucleobase of a second oligonucleotide or target nucleic acid when the first and second oligonucleotides are aligned is a mismatch or non-complementary nucleobase.

“Modulating” refers to changing or adjusting a feature in a cell, tissue, organ or organism. For example, modulating IRF5 RNA can mean to increase or decrease the level of IRF5 RNA and/or IRF5 protein in a cell, tissue, organ or organism. A “modulator” effects the change in the cell, tissue, organ or organism. For example, an IRF5 compound can be a modulator that decreases the amount of IRF5 RNA and/or IRF5 protein in a cell, tissue, organ or organism.

“MOE” means methoxyethyl.

“Monomer” refers to a single unit of an oligomer. Monomers include, but are not limited to, nucleosides and nucleotides.

“Motif” means the pattern of unmodified and/or modified sugar moieties, nucleobases, and/or internucleoside linkages, in an oligonucleotide.

“Natural” or “naturally occurring” means found in nature.

“Non-bicyclic modified sugar” or “non-bicyclic modified sugar moiety” means a modified sugar moiety that comprises a modification, such as a substituent, that does not form a bridge between two atoms of the sugar to form a second ring.

“Nucleic acid” refers to molecules composed of monomeric nucleotides. A nucleic acid includes, but is not limited to, ribonucleic acids (RNA), deoxyribonucleic acids (DNA), single-stranded nucleic acids, and double-stranded nucleic acids.

“Nucleobase” means a heterocyclic moiety capable of pairing with a base of another nucleic acid. As used herein a “naturally occurring nucleobase” is adenine (A), thymine (T), cytosine (C), uracil (U), and guanine (G). A “modified nucleobase” is a naturally occurring nucleobase that is chemically modified. A “universal base” or “universal nucleobase” is a nucleobase other than a naturally occurring nucleobase and modified nucleobase, and is capable of pairing with any nucleobase.

“Nucleobase sequence” means the order of contiguous nucleobases in a nucleic acid or oligonucleotide independent of any sugar or internucleoside linkage.

“Nucleoside” means a compound comprising a nucleobase and a sugar moiety. The nucleobase and sugar moiety are each, independently, unmodified or modified. “Modified nucleoside” means a nucleoside comprising a modified nucleobase and/or a modified sugar moiety. Modified nucleosides include abasic nucleosides, which lack a nucleobase.

“Oligomeric compound” means a compound comprising a single oligonucleotide and, optionally, one or more additional features, such as a conjugate group or terminal group.

“Oligonucleotide” means a polymer of linked nucleosides each of which can be modified or unmodified, independent one from another. Unless otherwise indicated, oligonucleotides consist of 8-80 linked nucleosides. “Modified oligonucleotide” means an oligonucleotide, wherein at least one sugar, nucleobase, or internucleoside linkage is modified. “Unmodified oligonucleotide” means an oligonucleotide that does not comprise any sugar, nucleobase, or internucleoside modification.

“Parent oligonucleotide” means an oligonucleotide whose sequence is used as the basis of design for more oligonucleotides of similar sequence but with different lengths, motifs, and/or chemistries. The newly designed oligonucleotides may have the same or overlapping sequence as the parent oligonucleotide.

“Parenteral administration” means administration through injection or infusion. Parenteral administration includes subcutaneous administration, intravenous administration, intramuscular administration, intraarterial administration, intraperitoneal administration, or intracranial administration, e.g. intrathecal or intracerebroventricular administration.

“Participant” means the common term “subject”.

“Pharmaceutically acceptable carrier or diluent” means any substance suitable for use in administering to an individual. For example, a pharmaceutically acceptable carrier can be a sterile aqueous solution, such as PBS or water-for-injection.

“Pharmaceutically acceptable salts” means physiologically and pharmaceutically acceptable salts of compounds, such as oligomeric compounds or oligonucleotides, i.e., salts that retain the desired biological activity of the parent compound and do not impart undesired toxicological effects thereto.

“Pharmaceutical agent” means a compound that provides a therapeutic benefit when administered to an individual.

“Pharmaceutical composition” means a mixture of substances suitable for administering to an individual. For example, a pharmaceutical composition may comprise one or more compounds or salt thereof and a sterile aqueous solution.

“Phosphorothioate linkage” means a modified phosphate linkage in which one of the non-bridging oxygen atoms is replaced with a sulfur atom. A phosphorothioate internucleoside linkage is a modified internucleoside linkage.

“Phosphorus moiety” means a group of atoms comprising a phosphorus atom. In certain embodiments, a phosphorus moiety comprises a mono-, di-, or tri-phosphate, or phosphorothioate.

“Portion” means a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an oligomeric compound.

“Prevent” refers to delaying or forestalling the onset, development or progression of a disease, disorder, or condition for a period of time from minutes to indefinitely.

“Prodrug” means a compound in a form outside the body which, when administered to an individual, is metabolized to another form within the body or cells thereof. In certain embodiments, the metabolized form is the active, or more active, form of the compound (e.g., drug). Typically conversion of a prodrug within the body is facilitated by the action of an enzyme(s) (e.g., endogenous or viral enzyme) or chemical(s) present in cells or tissues, and/or by physiologic conditions.

“Reduce” means to bring down to a smaller extent, size, amount, or number.

“RefSeq No.” is a unique combination of letters and numbers assigned to a sequence to indicate the sequence is for a particular target transcript (e.g., target gene). Such sequence and information about the target gene (collectively, the gene record) can be found in a genetic sequence database. Genetic sequence databases include the NCBI Reference Sequence database, GenBank, the European Nucleotide Archive, and the DNA Data Bank of Japan (the latter three forming the International Nucleotide Sequence Database Collaboration or INSDC).

“Region” is defined as a portion of the target nucleic acid having at least one identifiable structure, function, or characteristic.

“RNAi compound” means an antisense compound that acts, at least in part, through RISC or Ago2, but not through RNase H, to modulate a target nucleic acid and/or protein encoded by a target nucleic acid. RNAi compounds include, but are not limited to double-stranded siRNA, single-stranded RNA (ssRNA), and microRNA, including microRNA mimics.

“Segments” are defined as smaller or sub-portions of regions within a nucleic acid.

“Side effects” means physiological disease and/or conditions attributable to a treatment other than the desired effects. In certain embodiments, side effects include injection site reactions, liver function test abnormalities, renal function abnormalities, liver toxicity, renal toxicity, central nervous system abnormalities, myopathies, and malaise. For example, increased aminotransferase levels in serum may indicate liver toxicity or liver function abnormality. For example, increased bilirubin may indicate liver toxicity or liver function abnormality.

“Single-stranded” in reference to a compound means the compound has only one oligonucleotide.

“Self-complementary” means an oligonucleotide that at least partially hybridizes to itself. A compound consisting of one oligonucleotide, wherein the oligonucleotide of the compound is self-complementary, is a single-stranded compound. A single-stranded compound may be capable of binding to a complementary compound to form a duplex.

“Sites” are defined as unique nucleobase positions within a target nucleic acid.

“Specifically hybridizable” refers to an oligonucleotide having a sufficient degree of complementarity between the oligonucleotide and a target nucleic acid to induce a desired effect, while exhibiting minimal or no effects on non-target nucleic acids. In certain embodiments, specific hybridization occurs under physiological conditions.

“Specifically inhibit” with reference to a target nucleic acid means to reduce or block expression of the target nucleic acid while exhibiting fewer, minimal, or no effects on non-target nucleic acids. Reduction does not necessarily indicate a total elimination of the target nucleic acid's expression.

“Standard cell assay” means assay(s) described in the Examples and reasonable variations thereof.

“Standard in vivo experiment” means the procedure(s) described in the Example(s) and reasonable variations thereof.

“Stereorandom chiral center” in the context of a population of molecules of identical molecular formula means a chiral center having a random stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center. The stereochemical configuration of a chiral center is considered random when it is the result of a synthetic method that is not designed to control the stereochemical configuration. In certain embodiments, a stereorandom chiral center is a stereorandom phosphorothioate internucleoside linkage.

“Study intervention” as used herein, refers to an antisense oligomeric compound targeted to an IRF5 nucleic acid (e.g., ION 729018), or a placebo.

“Sugar moiety” means an unmodified sugar moiety or a modified sugar moiety. As used herein, “unmodified sugar moiety” means a β-D-ribosyl moiety, as found in naturally occurring RNA, or a β-D-2′-deoxyribosyl sugar moiety as found in naturally occurring DNA. As used herein, “modified sugar moiety” or “modified sugar” means a sugar surrogate or a furanosyl sugar moiety other than a β-D-ribosyl or a β-D-2′-deoxyribosyl. Modified furanosyl sugar moieties may be modified or substituted at a certain position(s) of the sugar moiety, substituted, or unsubstituted, and they may or may not have a stereoconfiguration other than β-D-ribosyl. Modified furanosyl sugar moieties include bicyclic sugars and non-bicyclic sugars.

“Sugar surrogate” means a modified sugar moiety that does not comprise a furanosyl or tetrahydrofuranyl ring (is not a “furanosyl sugar moiety”) and that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

“Synergy” or “synergize” refers to an effect of a combination that is greater than additive of the effects of each component alone at the same doses.

“Target gene” refers to a gene encoding a target.

“Targeting” means the specific hybridization of a compound to a target nucleic acid in order to induce a desired effect.

“Target nucleic acid,” “target RNA,” “target RNA transcript” and “nucleic acid target” all mean a nucleic acid capable of being targeted by compounds described herein.

“Target region” means a portion of a target nucleic acid to which one or more compounds is targeted.

“Target segment” means the sequence of nucleotides of a target nucleic acid to which a compound is targeted. “5′ target site” refers to the 5′-most nucleotide of a target segment. “3′ target site” refers to the 3′-most nucleotide of a target segment.

“Terminal group” means a chemical group or group of atoms that is covalently linked to a terminus of an oligonucleotide.

“Therapeutically effective amount” means an amount of a compound, pharmaceutical agent, or composition that provides a therapeutic benefit to an individual.

“Treat” refers to administering a compound or pharmaceutical composition to effect an alteration or improvement of a disease, disorder, or condition in a subject.

### Certain Embodiments

Compositions and methods are described for inhibiting IRF5 expression in a human subject using compounds targeted to an IRF5 nucleic acid. In one embodiment, the IRF5 nucleic acid has the sequence set forth in RefSeq or GENBANK Accession No. U51127.1 (incorporated by reference, disclosed herein as SEQ ID NO: 4); GENBANK Accession No. NT_007933.14 truncated from nucleotides 53761170 to U.S. Pat. No. 53,774,065 (incorporated by reference, disclosed herein as SEQ ID NO. 2); GENBANK Accession No. DC427600.1 (incorporated by reference, disclosed herein as SEQ ID NO: 5); GENBANK Accession No. NM_001098627.3 (incorporated by reference, disclosed herein as SEQ ID NO: 1); GENBANK Accession No. NM_001098629.2 (incorporated by reference, disclosed herein as SEQ ID NO: 3); GENBANK Accession No. NM_001098630.2 (incorporated by reference, disclosed herein as SEQ ID NO: 6); GENBANK Accession No. NM_001242452.2 (incorporated by reference, disclosed herein as SEQ ID NO: 7); GENBANK Accession No. NM_032643.4 (incorporated by reference, disclosed herein as SEQ ID NO: 8); and GENBANK Accession No. NC_000007.14 truncated from nucleotides 128935001 to 128953000 (incorporated by reference, disclosed herein as SEQ ID NO: 9).

In one embodiment, the compound targeted to an IRF5 nucleic acid is an oligomeric compound. The oligomeric compound may comprise or consist of an oligonucleotide, which may be a modified oligonucleotide. In one embodiment, the oligonucleotide is 8, 9, 10, 11, 12, 13, 14, 15, 16, 17, 18, 19, 20, 21, 22, 23, 24, 25, 26, 27, 28, 29, or 30 linked subunits in length, or a range defined by any two of the above values—e.g., 8 to 80, 12 to 22, 12 to 30, 12 to 50, 13 to 30, 13 to 50, 14 to 20, 14 to 30, 14 to 50, 15 to 20, 15 to 30, 15 to 50, 16 to 20, 16 to 30, 16 to 50, 17 to 20, 17 to 30, 17 to 50, 18 to 20, 18 to 22, 18 to 24, 18 to 30, 18 to 50, 19 to 22, 19 to 30, 19 to 50, or 20 to 30 linked subunits. The linked subunits may be nucleotides, nucleosides, or nucleobases. The compound may also comprise additional features or elements, such as a conjugate group (e.g., that is attached to the oligonucleotide). In embodiments where a conjugate group comprises a nucleoside (i.e. a nucleoside that links the conjugate group to the oligonucleotide), the nucleoside of the conjugate group is not counted in the length of the oligonucleotide.

In a preferred embodiment, the oligomeric compound is an antisense compound comprising an oligonucleotide that includes a nucleobase sequence complementary to that of IRF5—i.e. having a nucleobase sequence that, when written in the 5′ to 3′ direction, comprises the reverse complement of a target segment of an IRF5 nucleic acid. In certain embodiments, the antisense activity involves a change in an amount of an IRF5 nucleic acid or encoded protein, or a change in the ratio of splice variants of an IRF5 nucleic acid or encoded protein. In certain antisense activities, the hybridization of the antisense compound to an IRF5 nucleic acid results in recruitment of a protein that cleaves the IRF5 nucleic acid. For example, certain compounds described herein result in RNase H mediated cleavage of an IRF5 nucleic acid. RNase H is a cellular endonuclease that cleaves the RNA strand of an RNA:DNA duplex. The DNA in such an RNA:DNA duplex need not be unmodified DNA. In certain embodiments, compounds described herein are sufficiently “DNA-like” to elicit RNase H activity. Further, in certain embodiments, one or more non-DNA-like nucleosides in the gap of a gapmer are tolerated.

In certain antisense activities, the antisense compound or a portion of the compound is loaded into an RNA-induced silencing complex (RISC), ultimately resulting in cleavage of an IRF5 nucleic acid. For example, certain compounds described herein result in cleavage of an IRF5 nucleic acid by an Argonaute protein. Compounds that are loaded into a RISC are RNAi compounds. The RNAi compounds may be double-stranded (siRNA) or single-stranded (ssRNA).

In certain embodiments, the hybridization of the antisense compound to an IRF5 nucleic acid results in the alteration of splicing of the IRF5 nucleic acid. In certain embodiments, hybridization of the antisense compound to an IRF5 nucleic acid results in inhibition of a binding interaction between the IRF5 nucleic acid and a protein or other nucleic acid. For example, hybridization of the compound to an IRF5 nucleic acid may result in alteration of translation of the IRF5 nucleic acid.

Non-complementary nucleobases between the antisense compound and an IRF5 nucleic acid may be tolerated, provided that the compound remains able to specifically hybridize to the IRF5 nucleic acid. Moreover, a compound may hybridize over one or more segments of an IRF5 nucleic acid such that intervening or adjacent segments are not involved in the hybridization event (e.g., a loop structure, mismatch or hairpin structure). In certain embodiments, the antisense compound or a specified portion thereof are at least, or are up to 70%, 80%, 85%, 86%, 87%, 88%, 89%, 90%, 91%, 92%, 93%, 94%, 95%, 96%, 97%, 98%, 99%, or 100% complementary to an IRF5 nucleic acid, or region, segment or portion thereof. In certain embodiments, the compounds provided herein, or a specified portion thereof, are 70% to 75%, 75% to 80%, 80% to 85%, 85% to 90%, 90% to 95%, 95% to 100%, or any number in between these ranges, complementary to an IRF5 nucleic acid, target region, target segment, or specified portion thereof.

In one embodiment, the compound comprises a portion with at least 8 contiguous nucleobases, and preferably comprises an 8, 9, 10, 11, 12, 13, 14, 15, or 16 contiguous nucleobases that are complementary to an IRF5 nucleic acid. In certain embodiments, the oligonucleotide is up to 11, 12, 13, 14, 15, 16, 17, 18, 19, or 20 nucleobases in length and comprises no more than 4, no more than 3, no more than 2, or no more than 1 non-complementary nucleobase(s) relative to an IRF5 nucleic acid, or region, segment or portion thereof. In certain embodiments, the oligonucleotide is up to 11, 12, 13, 14, 15, 16, 17, 18, 19, 20, 21, 22, 23, 24, 25, 26, 27, 28, 29, or 30 nucleobases in length, and comprises no more than 6, no more than 5, no more than 4, no more than 3, no more than 2, or no more than 1 non-complementary nucleobase(s) relative to an IRF5 nucleic acid, or region, segment or portion thereof.

The non-complementary nucleobases may be clustered or interspersed with complementary nucleobases and need not be contiguous to each other or to complementary nucleobases. The location of a non-complementary nucleobase or nucleobases may be at the 5′ end, 3′ end, or at an internal position of the compound. In certain embodiments, the compound comprises one or more mismatched nucleobases relative to an IRF5 nucleic acid which reduce antisense activity against the IRF5 nucleic acid, but activity against a non-target is reduced by a greater amount, thereby improving the selectivity of the compound. In certain embodiments, the mismatch is specifically positioned within an oligonucleotide having a gapmer motif. In certain embodiments, a non-complementary nucleobase is located in the wing segment of a gapmer oligonucleotide. In certain such embodiments, the mismatch is at position 1, 2, 3, 4, 5, 6, 7, or 8 from the 5′-end of the gap region. In certain such embodiments, the mismatch is at position 9, 8, 7, 6, 5, 4, 3, 2, 1 from the 3′-end of the gap region. In certain such embodiments, the mismatch is at position 1, 2, 3, or 4 from the 5′-end of the wing region. In certain such embodiments, the mismatch is at position 4, 3, 2, or 1 from the 3′-end of the wing region. In certain embodiments, the mismatch is specifically positioned within an oligonucleotide not having a gapmer motif. In certain such embodiments, the mismatch is at position 1, 2, 3, 4, 5, 6, 7, 8, 9, 10, 11, or 12 from the 5′-end of the oligonucleotide. In certain such embodiments, the mismatch is at position 1, 2, 3, 4, 5, 6, 7, 8, 9, 10, 11, or 12 from the 3′-end of the oligonucleotide.

The antisense compound may comprise a single-stranded or a double stranded oligonucleotide. A single-stranded oligonucleotide comprises a region that is complementary to IRF5. In one embodiment, the oligonucleotide may comprise a self-complementary nucleobase sequence. A double-stranded antisense compound comprises a first oligonucleotide that has a region complementary to IRF5, and a second oligonucleotide that has a region complementary to the first oligonucleotide. In one embodiment, the first oligonucleotide of the double stranded antisense compound may comprise or consist of a modified oligonucleotide, and the second oligonucleotide of the double-stranded antisense compound may be modified or unmodified. Either or both oligonucleotides of the double-stranded antisense compound may comprise a conjugate group. Either or both oligonucleotides of the double-stranded antisense compound may include non-complementary overhanging nucleosides. In one embodiment, the double-stranded antisense compound has a first oligonucleotide that is a modified oligonucleotide 16-30 linked nucleosides in length, and a second oligonucleotide that is a modified oligonucleotide 16-30 linked nucleosides in length.

It is possible to increase or decrease the length of a compound, such as an oligonucleotide, and/or introduce mismatch bases without eliminating activity. Woolf et al. Proc. Natl. Acad. Sci. USA 89:7305-7309 (1992); Gautschi et al. J. Natl. Cancer Inst. 93:463-471 (March 2001); Maher and Dolnick, Nuc. Acid. Res. 16:3341-3358 (1998). However, seemingly small changes in oligonucleotide sequence, chemistry and motif can make large differences in one or more of the many properties required for clinical development. Seth et al., J. Med. Chem. 52:10 (2009); Egli et al., J. Am. Chem. Soc. 133:16642 (2011).

In one embodiment, the antisense compound may comprise an oligonucleotide that is shortened or truncated. For example, a shortened or truncated antisense compound targeted to an IRF5 nucleic acid may have a single subunit deleted from the 5′ end (5′ truncation), or alternatively from the 3′ end (3′ truncation) of the oligonucleotide. Similarly, the oligonucleotide may have two subunits deleted from the 5′ end, or alternatively, may have two subunits deleted from the 3′ end. Alternatively, the deleted nucleosides may be dispersed throughout the compound.

In one embodiment, the antisense compound may comprise an oligonucleotide that is lengthened. When a single additional subunit is present in a lengthened compound, the additional subunit may be located at the 5′ or 3′ end of the oligonucleotide. When two or more additional subunits are present, the added subunits may be adjacent to each other. For example, a compound may have two subunits added to the 5′ end (5′ addition) or, alternatively, to the 3′ end (3′ addition) of the oligonucleotide. Alternatively, the added subunits may be dispersed throughout the compound.

In one embodiment, the oligonucleotide is an RNA oligonucleotide, in which a thymine nucleobase in the oligonucleotide is replaced by a uracil nucleobase. An RNA which contains a uracil in place of a thymidine in a DNA sequence would be considered identical to the DNA sequence, since both uracil and thymidine pair with adenine. Examples of single-stranded and double-stranded RNA oligonucleotides include interfering RNA compounds (RNAi), which include double-stranded RNA compounds (also referred to as short-interfering RNA or siRNA) and single-stranded RNAi compounds (or ssRNA). Such compounds work at least in part through the RISC pathway to degrade and/or sequester a target nucleic acid (thus, include microRNA/microRNA-mimic compounds). As used herein, the term siRNA is meant to be equivalent to other terms used to describe nucleic acid molecules that are capable of mediating sequence-specific RNAi, for example, short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically modified siRNA, post-transcriptional gene silencing RNA (ptgsRNA), and others known in the art. As used herein, the term “RNAi” is meant to be equivalent to other terms used to describe sequence-specific RNA interference, such as post transcriptional gene silencing, translational inhibition, or epigenetics.

In one embodiment, the antisense compound comprises a single-stranded RNAi (ssRNAi) that has at least an 8, 9, 10, 11, 12, 13, 14, 15, or 16 contiguous nucleobase portion complementary to a target segment of an IRF5 nucleic acid. In certain embodiments, the compound comprises a double-stranded oligonucleotide having a first strand that is an siRNA guide strand and a second strand that is an siRNA passenger strand. In certain such embodiments, the second strand of the compound is complementary to the first strand. In certain such embodiments, each strand of the compound is 16, 17, 18, 19, 20, 21, 22, or 23 linked nucleosides in length. In certain such embodiments, the first or second strand of the compound can comprise a conjugate group.

In one embodiment, the antisense compound comprises one or more modified nucleotides in which the 2′ position in the sugar contains a halogen (such as fluorine group; 2′-F) or contains an alkoxy group (such as a methoxy group; 2′-OMe). In certain embodiments, the compound comprises at least one 2′-F sugar modification and at least one 2′-OMe sugar modification. In certain embodiments, the at least one 2′-F sugar modification and at least one 2′-OMe sugar modification are arranged in an alternating pattern for at least 2, 3, 4, 5, 6, 7, 8, 9, 10, 11, 12, 13, 14, 15, 16, 17, 18, 19, or 20 contiguous nucleobases along a strand of the compound. In certain embodiments, the compound comprises one or more linkages between adjacent nucleotides other than a naturally-occurring phosphodiester linkage. Examples of such linkages include phosphoramide, phosphorothioate, and phosphorodithioate linkages. The compounds may also be chemically modified nucleic acid molecules as taught in U.S. Pat. No. 6,673,616. In other embodiments, the compound contains a capped strand, as disclosed, for example, by WO 00/63364, filed Apr. 19, 2000. In certain embodiments, the compound consists of 16, 17, 18, 19, 20, 21, 22, or 23 linked nucleosides. In certain embodiments, the compound can comprise a conjugate group.

In one embodiment, the antisense compound comprises a double-stranded oligonucleotide with a first strand that has a region complementary to IRF5 and that comprises an RNA oligonucleotide which has uracil (U) in place of thymine (T). In certain embodiments, the compound comprises one or more modified nucleotides in which the 2 position of the sugar contains a halogen (such as fluorine group; 2′-F) or contains an alkoxy group (such as a methoxy group; 2′-OMe). In certain embodiments, the compound comprises at least one 2′-F sugar modification and at least one 2′-OMe sugar modification. In certain embodiments, the at least one 2′-F sugar modification and at least one 2′-OMe sugar modification are arranged in an alternating pattern for at least 2, 3, 4, 5, 6, 7, 8, 9, 10, 11, 12, 13, 14, 15, 16, 17, 18, 19, or 20 contiguous nucleobases along a strand of the dsRNA compound. In certain embodiments, the compound comprises one or more linkages between adjacent nucleotides other than a naturally-occurring phosphodiester linkage, such as phosphoramide, phosphorothioate and/or phosphorodithioate linkages. The compounds may also be chemically modified nucleic acid molecules as taught in U.S. Pat. No. 6,673,616. In other embodiments, the compound contains one or two capped strands, as disclosed, for example, by WO 00/63364, filed Apr. 19, 2000.

In one embodiment, the antisense compound comprises a modified oligonucleotide including a gap segment consisting of linked deoxynucleosides—e.g., a 5′ wing segment consisting of linked nucleosides; and a 3′ wing segment consisting of linked nucleosides, wherein the gap segment is positioned immediately adjacent to and between the 5′ and 3′ wing segments. In certain embodiments, each nucleoside of each wing segment comprises a modified sugar.

In one embodiment, the antisense compound targets an IRF5 nucleic acid having the nucleobase sequence of SEQ ID NO: 2, and preferably nucleotides 11737-11752 of SEQ ID NO: 2. In certain embodiments, the compound targets within nucleotides 4366-4381, 5141-5156, 5140-5160, 5179-5194, 11544-11559, 11542-11596, 11736-11751, 11737-11752, 11720-11790, or 11794-11809 of SEQ ID NO: 2. In certain embodiments, the compound is single-stranded or double-stranded. In certain embodiments, the compound is a modified oligonucleotide 12 to 30 linked nucleosides in length, and preferably 16 to 30 linked nucleosides in length. In certain embodiments, the compound comprises a modified oligonucleotide that is at least 85%, at least 90%, at least 95%, or 100% complementary to nucleobases 4366-4381, 5141-5156, 5140-5160, 5179-5194, 11544-11559, 11542-11596, 11736-11751, 11737-11752, 11720-11790, or 11794-11809 of SEQ ID NO: 2. In certain such embodiments, the compound comprises at least an 8, 9, 10, 11, 12, 13, 14, 15, or 16 contiguous nucleobase portion complementary to an equal length portion within nucleotides 4366-4381, 5141-5156, 5140-5160, 5179-5194, 11544-11559, 11542-11596, 11736-11751, 11737-11752, 11720-11790, or 11794-11809 of SEQ ID NO: 2.

In one embodiment, the antisense compound comprises a modified oligonucleotide that is 12 to 30, preferably 16-30, and more preferably 16 nucleosides in length, and has a nucleobase sequence comprising at least an 8, 9, 10, 11, 12, 13, 14, 15, or 16 contiguous nucleobase portion any one of SEQ ID NOs: 10, 11, 12, 13, 14, 15, and 16 (Table 1). In certain embodiments, the modified oligonucleotide is 12 to 30, preferably 16-30, and more preferably 16 linked nucleosides in length, and has a nucleobase sequence comprising any one of SEQ ID NOs: 10, 11, 12, 13, 14, 15, and 16. In certain embodiments, the modified oligonucleotide has a nucleobase sequence consisting of any one of SEQ ID NOs: 10, 11, 12, 13, 14, 15, and 16.

TABLE 1
Antisense Oligonucleotide Sequences

| SEQ | | SEQ ID NO: 2 |
|---|---|---|
| ID | | Start Site- |
| NO: | Sequence | Stop Site |
| 10 | CCTATACAGCTAGGCC | 11544-11559 |
| 11 | TCTGATATGATACCTA | 11737-11752 |
| 12 | TATTTCTGCTCCAGGT | 11794-11809 |
| 13 | CTGATATGATACCTAA | 11736-11751 |
| 14 | ACGAGTTATGGGAAGG | 5141-5156 |
| 15 | ATGAGTAATAGGAGTG | 5179-5194 |
| 16 | TGTCTAGTGTCATGGA | 4366-4381 |

In one embodiment, the foregoing modified oligonucleotides may comprise at least one modified internucleoside linkage, at least one modified sugar, and/or at least one modified nucleobase. In certain such embodiments, the at least one modified sugar comprises a 2′-O-methoxyethyl group. In certain embodiments, at least one modified sugar is a bicyclic sugar, such as a 4′-CH(CH₃)—O-2′ group, a 4′-CH₂—O-2′ group, or a 4′-(CH₂)₂—O-2′ group. In certain embodiments, the modified oligonucleotide comprises at least one modified internucleoside linkage, such as a phosphorothioate internucleoside linkage. In certain embodiments, the modified oligonucleotide comprises at least one modified nucleobase, such as 5-methylcytosine.

In one embodiment, the antisense compound may comprise a pharmaceutically acceptable salt of the foregoing modified oligonucleotides, such as a sodium salt or a potassium salt.

In one embodiment, the antisense compound comprises or consists of a modified oligonucleotide that is 12-30 linked nucleobases in length, with a nucleobase sequence that comprises the sequence recited in any one of SEQ ID NOs: 10, 11, 13, 14, and 15. The modified oligonucleotide comprises: a gap segment consisting of ten linked deoxynucleosides, a 5′ wing segment consisting of three linked nucleosides, and a 3′ wing segment consisting of three linked nucleosides. The gap segment is positioned between the 5′ wing segment and the 3′ wing segment. Each nucleoside of the wing segments comprises a cEt sugar, each internucleoside linkage is a phosphorothioate linkage, and each cytosine is a 5-methylcytosine. In certain embodiments, the modified oligonucleotide consists of 16-30 linked nucleosides and, more preferably, 16 linked nucleosides.

In one embodiment, the antisense compound comprises or consists of a modified oligonucleotide 12-30 linked nucleobases in length having a nucleobase sequence comprising the sequence recited in SEQ ID NO: 12. The modified oligonucleotide comprises: a gap segment consisting of ten linked deoxynucleosides, a 5′ wing segment consisting of two linked nucleosides, and a 3′ wing segment consisting of four linked nucleosides. The gap segment is positioned between the 5′ wing segment and the 3′ wing segment. Each nucleoside in the 5′ wing segment comprises a cEt sugar. The nucleosides in the 3′ wing segment comprise from the 5′ to 3′ direction, a cEt sugar, a 2′-MOE sugar, a cEt sugar, and a 2′-MOE sugar. Each internucleoside linkage is a phosphorothioate linkage, and each cytosine is a 5-methylcytosine. In certain embodiments, the modified oligonucleotide consists of 16-30 linked nucleosides and, more preferably, 16 linked nucleosides.

In one embodiment, the antisense compound comprises or consists of a modified oligonucleotide 12-30 linked nucleobases in length having a nucleobase sequence comprising the sequence recited in any one of SEQ ID NOs: 12 and 16. The modified oligonucleotide comprises: a gap segment consisting of nine linked deoxynucleosides, a 5′ wing segment consisting of two linked nucleosides, and a 3′ wing segment consisting of five linked nucleosides. The gap segment is positioned between the 5′ wing segment and the 3′ wing segment. Each nucleoside in the 5′ wing segment comprises a cEt sugar. The nucleosides in the 3′ wing segment comprise from 5′ to 3′ direction, a 2′-MOE sugar, a 2′-MOE sugar, a 2′-MOE sugar, a cEt sugar and a cEt sugar. Each internucleoside linkage is a phosphorothioate linkage, and each cytosine is a 5-methylcytosine. In certain embodiments, the modified oligonucleotide consists of 16-30 linked nucleosides and, more preferably, 16 linked nucleosides.

In a preferred embodiment, the compound comprises or consists of ION 729018, which is a 3-10-3 constrained-ethyl (cEt) gapmer antisense oligonucleotide (ASO). cEt gapmer ASOs are known to confer higher stability and target RNA affinity, which results in better drug-like properties compared to other ASOs. ION 729018 consists of 16 nucleotides with a uniform phosphorothioated backbone and 3 nucleotides on both the 5′ and 3′ ends containing cEt-modified ribose. The chemical structure of ION 729018 is shown below:

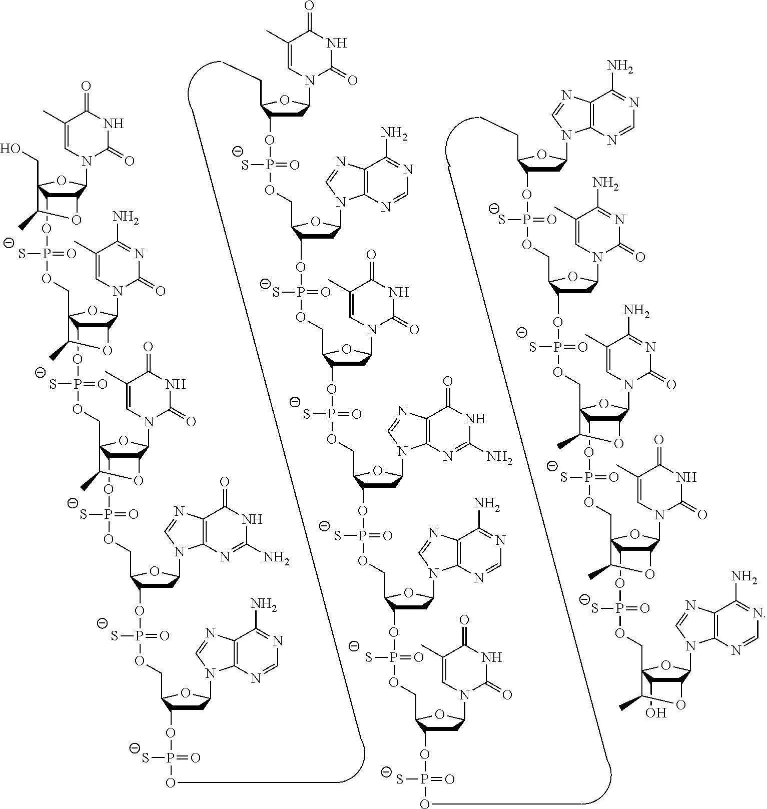

The compound may also comprise or consist of the sodium salt of ION 729018, having the following chemical structure:

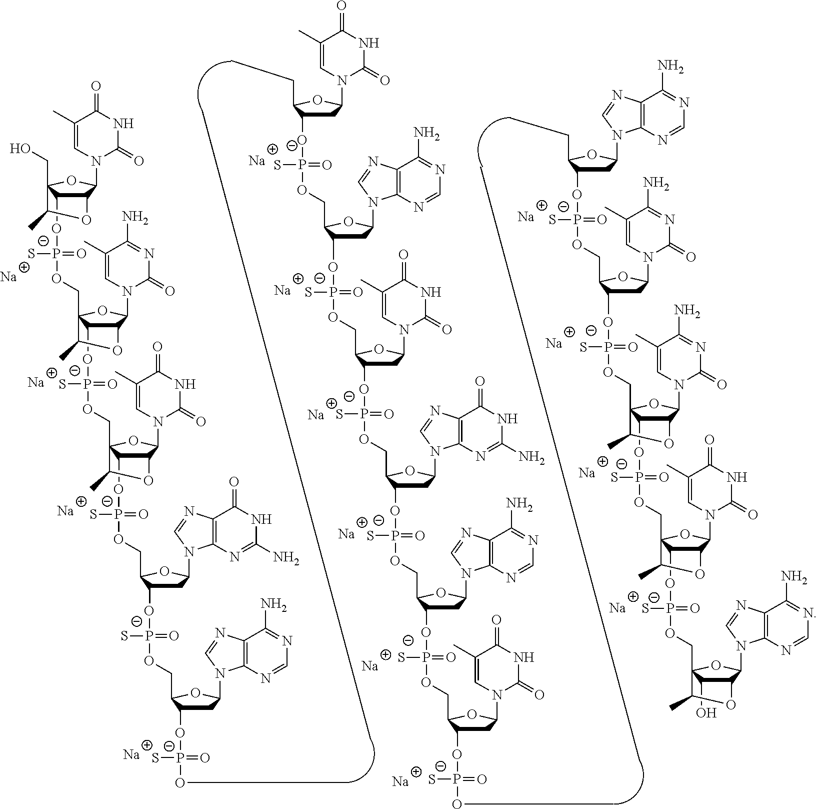

Studies support the use of ION-729018 as a particularly preferred compound for targeting IRF5 in the treatment of diseases associated with IRF5, such as IBD, including Crohn's disease and UC. ION-729018 targets human IRF5 messenger ribonucleic acid (mRNA), leading to degradation. ION-729018 has been shown to be selective and potent in targeting and reducing expression of human IRF5 mRNA and protein in human cell lines, in human primary peripheral blood mononuclear cells (PBMCs), and in primary human monocyte derived macrophages and dendritic cells. In particular, ION-729018 was tested for potential intrinsic immunostimulatory properties in an in vitro PBMC activation assay. Treatment of human PBMCs isolated from 4 different healthy donors with increasing concentrations of ION-729018 was found to elicit similar or less cytokine production than the negative control. ION-729018 was also shown to reduce IRF5 RNA and protein, and anti-inflammatory properties in stimulated human monocyte derived macrophages and dendritic cells—specifically, lowered secretion of IL-23 and interferon-γ-induced protein 10, and reduced inflammatory and interferon-dependent gene expression. Analyses of ION-729018 binding to SEMA3A and STAT4 RNA transcripts in comparison to IRF5, suggests minimal impact of off-target binding. ION-729018 was also found to decrease the expression of IRF5 RNA in an in vivo transgenic mouse model.

In one embodiment, the antisense compound targeted to an IRF5 nucleic acid is used in the manufacture or preparation of a composition or medicament for treating a human subject having, or at risk of having a disease associated with IRF5. In certain embodiments, the composition comprises or consists of a compound that is an IRF5 specific inhibitor, and preferably is an antisense compound targeted to IRF5. In certain embodiments, the compound inhibits expression of IRF5 in the gastrointestinal tract, liver, lungs, kidneys, and/or joints. In certain embodiments, the disease is an inflammatory disease. In certain embodiments, the disease is a gastrointestinal disease. In certain embodiments, the gastrointestinal disease is ulcerative colitis or Crohn's disease. In certain embodiments, the disease is inflammatory bowel disease (IBD), systemic lupus erythematosus (SLE), rheumatoid arthritis, primary biliary cirrhosis, systemic sclerosis, Sjogren's syndrome, multiple sclerosis, scleroderma, interstitial lung disease (SSc-ILD), polycystic kidney disease (PKD), chronic kidney disease (CKD), nonalcoholic steatohepatitis (NASH), liver fibrosis, asthma, or severe asthma. In certain embodiments, the medicament is used for treating inflammation, cirrhosis, fibrosis, proteinuria, joint inflammation, autoantibody production, inflammatory cell infiltration, collagen deposits, or inflammatory cytokine production in an individual having, or at risk of having, a disease associated with IRF5. In certain embodiments, the medicament is used for treating inflammation in the gastrointestinal tract, diarrhea, pain, fatigue, abdominal cramping, blood in the stool, intestinal inflammation, disruption of the epithelial barrier of the gastrointestinal tract, dysbiosis, increased bowel frequency, tenesmus or painful spasms of the anal sphincter, constipation, or unintended weight loss in an individual having, or at risk of having, a disease associated with IRF5.

In one embodiment, a method of inhibiting expression of IRF5 in an individual having, or at risk of having a disease associated with IRF5, comprises administering to the individual a composition comprising or consisting of a compound targeted to an IRF5 nucleic acid to inhibit expression of IRF5 in the individual. In certain embodiments, the compound is an IRF5 specific inhibitor, and preferably is an antisense compound targeted to IRF5. In certain embodiments, the compound inhibits expression of IRF5 in the gastrointestinal tract, liver, lungs, kidneys, and/or joints. In certain embodiments, the disease is an inflammatory disease. In certain embodiments, the disease is a gastrointestinal disease. In certain embodiments, the gastrointestinal disease is ulcerative colitis or Crohn's disease. In certain embodiments, the individual has, or is at risk of having, inflammatory bowel disease (IBD), systemic lupus erythematosus (SLE), rheumatoid arthritis, primary biliary cirrhosis, systemic sclerosis, Sjogren's syndrome, multiple sclerosis, scleroderma, interstitial lung disease (SSc-ILD), polycystic kidney disease (PKD), chronic kidney disease (CKD), NASH, liver fibrosis, asthma, or severe asthma. In certain embodiments, the individual has, or is at risk of having, inflammation, cirrhosis, fibrosis, proteinuria, joint inflammation, autoantibody production, inflammatory cell infiltration, collagen deposits, or inflammatory cytokine production. In certain embodiments, the individual has, or is at risk of having, inflammation in the gastrointestinal tract, diarrhea, pain, fatigue, abdominal cramping, blood in the stool, intestinal inflammation, disruption of the epithelial barrier of the gastrointestinal tract, dysbiosis, increased bowel frequency, tenesmus or painful spasms of the anal sphincter, constipation, or unintended weight loss.

In one embodiment, the antisense compound may be administered parenterally. Parenteral administration includes subcutaneous administration, intravenous administration, intramuscular administration, intraarterial administration, intraperitoneal administration, or intracranial administration, e.g. intrathecal or intracerebroventricular administration. In certain embodiments, the compound is administered through injection or infusion.

In one embodiment, the antisense compound may be administered orally. In certain embodiments, the antisense compound is provided as an active pharmaceutical ingredient (API) supplied in capsules for oral administration.

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the scope of the invention.

In some embodiments, the antisense oligomer described herein is administered in an amount of about 20-1200 mg per dose administration. In some embodiments, the antisense oligomer described herein is administered in an amount of about 20-50 mg, 50-75 mg, 75-100 mg, 100-125 mg, 100-130 mg, 110-120 mg, 115-130 mg, 125-150 mg, 150-175 mg, 175-200 mg, 200-225 mg, 225 250 mg, 250-275 mg, 275-300 mg, 300-325 mg, 325-350 mg, 350-375 mg, 375-400 mg, 25-75 mg, 50-100 mg, 100-150 mg, 150-200 mg, 200-250 mg, 250-300 mg, 300-350 mg, 350-400 mg, 25-100 mg, 50-150 mg, 100-200 mg, 150-250 mg, 200-300 mg, 300-400 mg, 400-500 mg, 500-600 mg, 600-700 mg, 700-800 mg, 800-900 mg, 900-1000 mg, 1000-1100 mg, or 1100-1200 mg per dose administration. In some embodiments, the antisense oligomer described herein is administered in an amount of about 120 mg, about 240 mg, about 360 mg, about 480 mg, about 600 mg, about 720 mg, about 840 mg, about 940 mg, about 1060 mg, or about 1200 mg per dose administration. In some embodiments, the antisense oligomer described herein is administered in an amount of about 100-200 mg per dose administration, about 300-400 mg per dose administration, about 650-850 mg per dose administration, or about 1000-1200 mg per dose administration. In some embodiments, the antisense oligomer described herein is administered in an amount of about 20 mg per dose administration. In some embodiments, the antisense oligomer described herein is administered in an amount of about 30 mg per dose administration. In some embodiments, the antisense oligomer described herein is administered in an amount of about 60 mg per dose administration. In some embodiments, the antisense oligomer described herein is administered in an amount of about 120 mg per dose administration. In some embodiments, the antisense oligomer described herein is administered in an amount of about 240 mg per dose administration. In some embodiments, the antisense oligomer described herein is administered in an amount of about 360 mg per dose administration. In some embodiments, the antisense oligomer described herein is administered in an amount of about 480 mg per dose administration. In some embodiments, the antisense oligomer described herein is administered in an amount of about 600 mg per dose administration. In some embodiments, the antisense oligomer described herein is administered in an amount of about 720 mg per dose administration. In some embodiments, the antisense oligomer described herein is administered in an amount of about 840 mg per dose administration. In some embodiments, the antisense oligomer described herein is administered in an amount of about 960 mg per dose administration. In some embodiments, the antisense oligomer described herein is administered in an amount of about 1080 mg per dose administration. In some embodiments, the antisense oligomer described herein is administered in an amount of about 1200 mg per dose administration.

In some embodiments, the antisense oligomer described herein is administered in an amount of about 20-1200 mg per daily administration. In some embodiments, the antisense oligomer described herein is administered in an amount of about 20-50 mg, 50-75 mg, 75-100 mg, 100-125 mg, 100-130 mg, 110-120 mg, 115-130 mg, 125-150 mg, 150-175 mg, 175-200 mg, 200-225 mg, 225 250 mg, 250-275 mg, 275-300 mg, 300-325 mg, 325-350 mg, 350-375 mg, 375-400 mg, 25-75 mg, 50-100 mg, 100-150 mg, 150-200 mg, 200-250 mg, 250-300 mg, 300-350 mg, 350-400 mg, 25-100 mg, 50-150 mg, 100-200 mg, 150-250 mg, 200-300 mg, 300-400 mg, 400-500 mg, 500-600 mg, 600-700 mg, 700-800 mg, 800-900 mg, 900-1000 mg, 1000-1100 mg, or 1100-1200 mg per daily administration. In some embodiments, the antisense oligomer described herein is administered in an amount of about 120 mg, about 240 mg, about 360 mg, about 480 mg, about 600 mg, about 720 mg, about 840 mg, about 940 mg, about 1060 mg, or about 1200 mg per daily administration. In some embodiments, the antisense oligomer described herein is administered in an amount of about 100-200 mg per daily administration, about 300-400 mg per daily administration, about 650-850 mg per daily administration, or about 1000-1200 mg per daily administration. In some embodiments, the antisense oligomer described herein is administered in an amount of about 20 mg per daily administration. In some embodiments, the antisense oligomer described herein is administered in an amount of about 30 mg per daily administration. In some embodiments, the antisense oligomer described herein is administered in an amount of about 60 mg per daily administration. In some embodiments, the antisense oligomer described herein is administered in an amount of about 120 mg per daily administration. In some embodiments, the antisense oligomer described herein is administered in an amount of about 240 mg per daily administration. In some embodiments, the antisense oligomer described herein is administered in an amount of about 360 mg per daily administration. In some embodiments, the antisense oligomer described herein is administered in an amount of about 480 mg per daily administration. In some embodiments, the antisense oligomer described herein is administered in an amount of about 600 mg per daily administration. In some embodiments, the antisense oligomer described herein is administered in an amount of about 720 mg per daily administration. In some embodiments, the antisense oligomer described herein is administered in an amount of about 840 mg per daily administration. In some embodiments, the antisense oligomer described herein is administered in an amount of about 960 mg per daily administration. In some embodiments, the antisense oligomer described herein is administered in an amount of about 1080 mg per daily administration. In some embodiments, the antisense oligomer described herein is administered in an amount of about 1200 mg per daily administration.

In some embodiments, the antisense oligomer described herein is administered once daily. In some embodiments, the antisense oligomer described herein is administered twice daily. In some embodiments, the antisense oligomer described herein is administered three times daily.

In some embodiments, the antisense oligomer described herein is administered in about 1 day, 2-day, 3-day, 4-day, 5-day, 6-day, 7-day, about 14-day, about 21-day or about 28-day intervals. In some embodiments, the antisense oligomer described herein is administered in 2-day intervals or about 2-day intervals. In some embodiments, the antisense oligomer described herein is administered in 3-day intervals or about 3-day intervals. In some embodiments, the antisense oligomer described herein is administered in 4-day intervals or about 4-day intervals. In some embodiments, the antisense oligomer described herein is administered in 5-day intervals or about 5-day intervals. In some embodiments, the antisense oligomer described herein is administered in 6-day intervals or about 6-day intervals. In some embodiments, the antisense oligomer described herein is administered in 7-day intervals or about 7-day intervals (i.e., Q1W). In some embodiments, the antisense oligomer described herein is administered in 14-day intervals or about 14-day intervals (i.e., Q2W). In some embodiments, In some embodiments, the antisense oligomer described herein is administered in an amount of about 120 mg, 240 mg, 360 mg, 480 mg, 600 mg, 720 mg, 840 mg, 960 mg, 1080 mg, 1200 mg, 1320 mg, 1440 mg, 1560 mg, 1680 mg, 1800 mg, 1920 mg, 2040 mg, 2160 mg, 2280 mg or 2400 mg per dose administration in 2-day interval, 3-day intervals, 4-day intervals, 5-day intervals, 6-day intervals, 7-day intervals, or 14-day intervals.

In some embodiments, the antisense oligomer described herein is administered for a duration of about 1 to 6 weeks. In some embodiments, the antisense oligomer described herein is administered for a duration of at least about 1 week, at least about 2 weeks, at least about 3 weeks, at least about 4 weeks, at least about 5 weeks, or at least about 6 weeks. In some embodiments, the antisense oligomer described herein is administered for a duration of at least about 1 week. In some embodiments, the antisense oligomer described herein is administered for a duration of at least about 2 weeks. In some embodiments, the antisense oligomer described herein is administered for a duration of at least about 3 weeks. In some embodiments, the antisense oligomer described herein is administered for a duration of at least about 4 weeks. In some embodiments, the antisense oligomer described herein is administered for a duration of at least about 5 weeks. In some embodiments, the antisense oligomer described herein is administered for a duration of at least about 6 weeks.

In some embodiments, the antisense oligomer described herein is administered for a treatment cycle of about 1 to 6 weeks. In some embodiments, the antisense oligomer described herein is administered for a treatment cycle of at least about 1 week, at least about 2 weeks, at least about 3 weeks, at least about 4 weeks, at least about 5 weeks, or at least about 6 weeks. In some embodiments, the antisense oligomer described herein is administered for a treatment cycle of at least about 1 week. In some embodiments, the antisense oligomer described herein is administered for a treatment cycle of at least about 2 weeks. In some embodiments, the antisense oligomer described herein is administered for a treatment cycle of at least about 3 weeks. In some embodiments, the antisense oligomer described herein is administered for a treatment cycle of at least about 4 weeks. In some embodiments, the antisense oligomer described herein is administered for a treatment cycle of at least about 5 weeks. In some embodiments, the antisense oligomer described herein is administered for a treatment cycle of at least about 6 weeks.

In some embodiments, the treatment cycle can be repeated. In some embodiments, the treatment cycle can be repeated for 2 times, 3 times, 4 times, 5 times, or 6 times. In some embodiments, the treatment method described herein further comprises providing the human subject with a treatment break before the treatment cycle is repeated. In some embodiments, the treatment break is about 1 week, 2 weeks, 3 weeks, 4 weeks, 5 weeks, or 6 weeks. In some embodiments, the treatment break is at least about 1 week, at least about 2 weeks, at least about 3 weeks, at least about 4 weeks, at least about 5 weeks, or at least about 6 weeks. In some embodiments, the treatment break is about 1 week or at least about 1 week. In some embodiments, the treatment break is about 2 weeks or at least about 2 weeks. In some embodiments, the treatment break is about 3 weeks or at least about 3 weeks. In some embodiments, the treatment break is about 4 weeks or at least about 4 weeks. In some embodiments, the treatment break is about 5 weeks or at least about 5 weeks. In some embodiments, the treatment break is about 6 weeks or at least about 6 weeks.

In some embodiments, the human subject is in a fast, semi-fasted, modified fasted, or fed conditions when the antisense oligomer is administered. In some embodiments, the human subject is in a fast condition when the antisense oligomer is administered. In some embodiments, the human subject is in a semi-fasted condition when the antisense oligomer is administered. In some embodiments, the human subject is in a modified fasted condition when the antisense oligomer is administered. In some embodiments, the human subject is in a fed condition when the antisense oligomer is administered. In some embodiments, for a semi-fasted condition, patients are fast overnight for at least 10 hours and continue fasting until a standard meal is served at 1 to 2 hours postdose on Day 1 to Day 14. Patients are not allowed to drink water from 1 hour prior to dosing until 1 hour after dosing. In some embodiments, for a modified fasted condition, no food is consumed for approximately 1 h, 2 h, 3 h, 4 h, 5 h, 6 h, 7 h, 8 h, 9 h, 10 h, 11 h, or 12 h before and approximately 1 h, 2 h, 3 h, 4 h, 5 h, 6 h, 7 h, 8 h, 9 h, 10 h, 11 h, or 12 h after study intervention administration on all days of dosing. Doses can be taken with approximately 240 mL of non carbonated water. An additional 60 mL of water may be ingested for the doses>720 mg. If fed, patients consume a standard meal 30 minutes before dosing on Day 1 to Day 14.

### EXAMPLES

### Example 1—ASO Study Design

A randomized, double-blind, placebo-controlled study is conducted to evaluate the single and multiple dose safety, tolerability, pharmacokinetics (PK), pharmacodynamics (PD), and immunogenicity of an antisense oligomer (ASO) targeted to an IRF5 nucleic acid. These data allow an assessment of safety and effectiveness of the ASO, assist in the development of dosage adjustment guidance and the determination of a potential dose regimen, and provide confirmation of the mechanism of action in treating diseases associated with IRF5, such as UC. The placebo control is used to establish the frequency and magnitude of changes in safety endpoints that may occur in the absence of active intervention. Randomization is used to minimize bias in the assignment of participants to intervention groups, and to increase the likelihood that known and unknown participant attributes (e.g., demographic and baseline characteristics) are evenly balanced across intervention groups. Blinded intervention is used to reduce potential bias during data collection and evaluation of safety endpoints by the investigator.

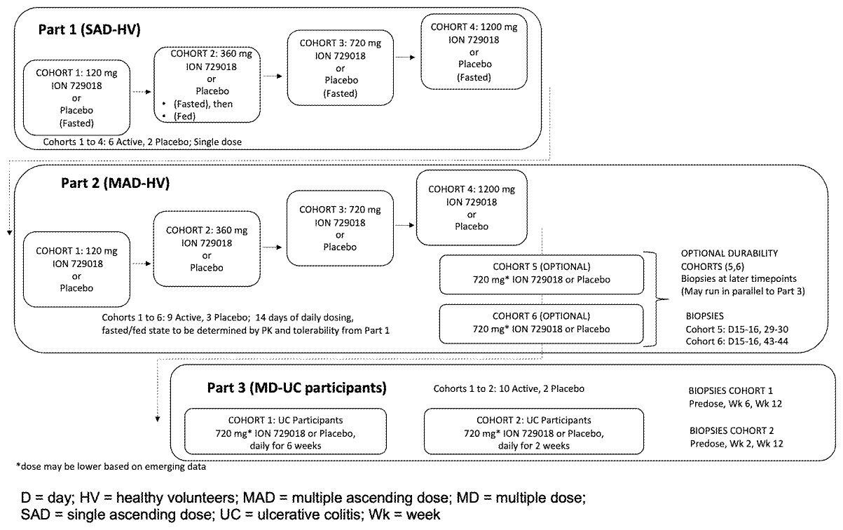

FIG. 1 shows a schematic diagram of the study, which consists of three parts. Part 1 assesses ASO administered orally as a single ascending dose (SAD), including an assessment of the administration of ASO under fasted and fed conditions to evaluate the impact of food on the pharmacokinetics of ASO and decrease the variability of data across participants. Part 2 assesses ASO administered orally as multiple ascending doses (MAD). Participants in Part 1 and Part 2 are healthy male and female subjects of non-childbearing potential between 18 to 60 years. The healthy participants allow the characterization of safety, tolerability, and PK for ASO without potential confounding information from underlying disease and concomitant medications that exist and occur in a patient population.

Part 3 assesses multiple doses (MD) of ASO administered orally in participants with moderately to severely active UC. Participants in Part 3 are male and female subjects with UC between 18 to 70 years, including women of childbearing potential. The UC participants have greater potential to demonstrate PK/PD effects in the patient population and to understand PK/PD differences between patients and healthy participants. The UC participants also allow the assessment of downstream effects of the study intervention, which are unlikely to show any changes in heathy participants.

Approximately 124-168 participants are enrolled in the study. The number of participants chosen for this study is consistent with those of similar designs and considered adequate to provide a preliminary safety assessment and PK assessment of ASO administered as single or multiple oral administrations in healthy participants, and as a multiple dose in participants with moderately to severely active UC. The sample size allows for a qualitative assessment of the food effect on ASO for the oral route of administration in Part 1 of the study, and for clinical UC assessments in Part 3 of this study.

Part 1 comprises approximately 28 participants in 2 cohorts of 8 subjects each, and 1 cohort (fasted-fed) of 12 subjects. The fasted-fed cohort assesses the effect of food on the systemic PK and stool concentrations of ASO. In one embodiment, Part 1 further comprises up to 2 additional cohorts of 8 and 12 subjects to assess safety or to assess a modified dose level within the existing low/high dose range.

Part 2 comprises approximately 48 to 72 participants in 4 escalating cohorts of 12 subjects each. Part 2 may further comprise up to 2 additional cohorts of 12 subjects each to assess durability—i.e. the change in tissue ASO concentration by dose level over time. Part 2 may also comprise up to 2 additional cohorts of 12 subjects each to assess safety or to assess a modified dose level within the existing low/high dose range.

Part 3 comprises approximately 24 participants in 2 cohorts of 12 subjects—1 cohort with daily dosing for 2 weeks, and 1 cohort with daily dosing for 6 weeks. The 2-week regimen allows comparison between participants with UC and healthy participants in Parts 1 and 2. The 6-week regimen is intended to achieve steady state tissue concentrations levels of ASO.

### Example 2—ASO Formulation And Dosage

The antisense oligomer ION-729018 was administered to study participants. Nonclinical studies suggest that ION-729018 has low toxicity and is well tolerated. Pharmacologic studies were conducted to determine the potential effects of ION-729018 on cardiovascular and respiratory parameters. No adverse effects were observed in telemetered conscious cynomolgus monkeys, following subcutaneous (SC) doses up to 30 mg/kg. The plasma concentration at 6 hours following the 30 mg/kg SC dose was 42,787 ng/mL. Functional observational battery (FOB) evaluations were conducted in a 13-week study in mice to determine the effects of ION-729018 on the central nervous system (CNS). No CNS-related effects at any dose. The No-Observed Adverse-Effect Level (NOAEL) for FOB evaluations was 75 mg/kg/week, which was associated with a maximum observed plasma concentration during a dosing interval (Cₘₐₓ) of 122,000 ng/mL and plasma concentration-time curve (AUC_{0-24 h}) of 206,000 ng-h/mL.

No evidence of ION-729018 toxicity was found in studies of oral administration in mice and cynomolgus monkeys where the NOAELs were the highest doses tested. In 13-week oral toxicity studies the No Adverse Effect Level (NOAEL) in mice was 500 mg/kg/day in mice (Cₘₐₓ: 8,900 ng/mL; AUC_{0-24 h}: 7,200 ng-h/mL), and 100 mg/kg/day in monkeys (Day1 Cₘₐₓ and AUC_{0-24 h}: 1,340 ng/mL and 8,820 ng-h/mL, respectively; Day 92 Cₘₐₓ and AUC_{0-24 h}: 1,450 ng/mL and 16,000 ng-h/mL, respectively).

No evidence of ION-729018 toxicity was found in the subcutaneous (SC) administration study in cynomolgus monkeys whereas liver toxicity was observed at the highest dose in the SC study in mice. In monkey 13-week SC toxicity study, the NOAEL was 30 mg/kg/week, the highest dose tested which corresponded to mean Cₘₐₓ and AUC_{0-24 h} values of 86,600 ng/mL and 642,000 ng-h/mL on Day 1 and 68,800 ng/mL and 742,000 ng-h/mL on Day 92, respectively. Minimal to mild transient changes in complement Bb and C3 and prolongation in activated partial thromboplastin time were found, but were considered nonadverse based on the low magnitude of change and transient nature. Microscopic evidence of ASO uptake in various tissues and organs was observed, but was not associated with degenerative or inflammatory changes, and therefore were considered nonadverse. Changes were partially to fully reversed after a 13-week recovery period.

In mouse 13-week SC toxicity study, the NOAEL was 25 mg/kg/week, which corresponded to Cₘₐₓ and AUC_{0-24 h} values of 36,600 ng/mL and 60,600 ng-h/mL, respectively. Minimal to mild hepatocyte necrosis was found at 75 mg/kg/week, which was associated with increases in alanine aminotransferase (ALT), aspartate aminotransferase (AST), and lactate dehydrogenase (LDH). Microscopic evidence of ASO uptake was observed, but was not associated with degenerative or inflammatory changes, and therefore was considered nonadverse. Changes were fully reversed after a 13-week recovery period.

ION-729018 was tested in a fertility and embryo-fetal developmental toxicity study in the mouse. There were no adverse effects on fertility, reproductive performance, or developmental toxicity at the doses tested in the study. The NOAEL was 21.4 mg/kg every other day or 75 mg/kg/week. ION-729018 was tested in an embryo-fetal developmental toxicity study in the rabbit. There was no evidence of developmental toxicity at the doses tested in the study. The NOAEL was 15 mg/kg every other day or 52.5 mg/kg/week.

The pharmacokinetics and metabolism profile of ION-729018 was evaluated in mice and monkeys. Plasma exposure to ION-729018 was increased with increasing oral doses in an approximately dose proportional manner. In monkeys, a slight accumulation (<2-fold increase in AUC) was observed on repeated oral dosing (not evaluated in mouse). The plasma half-life ranged from 2 to 11 hours in monkey after oral dosing (not determined in mouse). Colon tissue concentrations of ION-729018 increased with increasing dose on Day 1 (mice) and Day 92 (mice and monkeys) after oral and SC administrations, and were still detectable after the recovery period. Colon tissue mean half-lives were estimated to range from 254 to 396 hours (10.6 to 16.5 days) in mice and monkeys. In vitro plasma protein binding of ION-729018 (0.5, 5, and 150 pg/mL) was high (>96.75%) and concentration independent for the 2 nonclinical safety species (mouse, monkey) and human. An assessment of the metabolic stability and profile of ION-729018 in mouse, rat, and human feces homogenates indicated that ION-729018 was stable in mouse fecal homogenates, with 86% of unchanged drug (UD) remaining after incubation for 24 hours. However, some degradation was seen in rat and human fecal homogenates, with 77% and 38% UD remaining after 24 hours. The estimated t 1 of ION-729018 in human feces homogenates was 18 hours. Metabolites were 4-mer to 11-mer fragments of ION-729018, caused by hydrolysis of the thiophosphate linkage from both 3′-deletion and 5′-deletion. No human-specific metabolites were found.

The NOAEL doses from the 13-week oral toxicity studies in mouse and monkeys were converted for use in the study, by normalization of the doses to body surface area according to the Human Equivalent Dose (HED) approach proposed by the Food and Drug Administration (FDA). A safety factor of 10 was applied, which is generally considered to provide an appropriate margin of safety to protect human participants receiving the initial clinical dose. A maximum recommended starting dose (MRSD) is calculated in mg/kg/day and adjusted to an individual weighing 60 kg.

Based on a NOAEL of 500 mg/kg/day for mouse (oral daily dosing for 13 weeks) the HED is 40 mg/kg (2,400 mg for a 60-kg adult). With a safety factor of 10, the MRSD is 4 mg/kg (240 mg for a 60-kg adult). Based on a NOAEL of 100 mg/kg/day for monkeys (oral daily dosing for 13 weeks), the HED is 32 mg/kg (1920 mg for a 60-kg adult). With a safety factor of 10, the MRSD is 3.2 mg/kg (192 mg for a 60-kg adult). A starting dose of 120 mg is expected to provide a 20- and 16-fold dose margin compared to the NOAEL doses in the 13-week toxicity study in mouse and monkeys, respectively. Dose levels higher than 1200 mg are not permitted in either Part 1 or Part 2 of the study. Further, no levels that exceed the exposure ceilings set by the NOAEL are assessed.

ION-729018 was administered to study participants as a granulated active pharmaceutical ingredient (API) supplied in 120 mg capsules for oral administration. Antisense oligonucleotides are historically known to have a low rate of absorption when administered orally. ION-729018, administered via the oral route in this study, is intended to deliver therapeutic drug levels directly to the gastrointestinal tract with limited systemic exposure in order to reduce potential systemic toxicities. The placebo contains silicified microcrystalline cellulose filler alone equivalent to a 120 mg capsule. The compositions of the ASO and the matching placebo are described Table 2.

TABLE 2
ASO Compositions

| | ION-729018 | Placebo |
|---|---|---|
| Formulation | enteric protective capsule | enteric protective capsule |
| | | (identical to ASO capsule) |
| Unit Dose | ASO supplied as a 120 mg | placebo is silicified |
| Strength(s) | capsule | microcrystalline cellulose |
| | | filler, supplied as a 120 mg |
| | | capsule (identical to ASO |
| | | capsules) |
| Route Of | oral | oral |
| Administration | | |
| Excipients | Filler - silicified | Filler - silicified |
| | microcrystalline cellulose | microcrystalline cellulose |
| | Capsule shell (enteric | Capsule shell (enteric |
| | protection) - hypromellose | protection) - hypromellose |
| | acetate succinate | acetate succinate |
| | Sealing solution - 96% | Sealing solution - 96% |
| | ethanol/purified water | ethanol/purified water |

### Example 3—ASO Study, Part 1

In Part 1, single oral doses of ASO ranging from about 120 mg to about 1200 mg are assessed in healthy volunteers. Cohort 1 (120 mg), Cohort 3 (720 mg), and Cohort 3 (1200 mg) each consist of 8 participants that receive either a single oral dose of ASO or a placebo on Day 1, in a fasted condition (i.e. after an overnight fast of at least 10 hours)—6 subjects that receive ASO, and 2 subjects that receive placebo. Cohort 2 (360 mg) consists of 12 participants that receive either a single oral dose of ASO or a placebo in a fasted condition on Day 1 and in a fed condition on Day 7 (i.e. after ingestion of a standard high fat meal)—9 subjects that receive ASO, and 3 subjects that receive placebo. Part 1 preferably includes at least 8 female participants.

Participants are screened up to 28 days prior to dose administration. Day 1 is defined as the initial dose administration. Cohort 1 and Cohort 3 have an in-house period from Day −2 to Day 6, and an outpatient follow-up visit on Day 14. Cohort 2 has an in-house period from Day −2 to Day 12 with ASO administered on Day 1 and Day 7, and an outpatient follow-up visit on Day 20. The duration of the study for most participants in Cohort 1 and Cohort 3 is approximately 6 weeks, including the 4 week screening period and 2 weeks post-administration of the ASO. The study duration for Cohort 2 (fasted-fed cohort) is approximately 7 weeks, including the 4 week screening period followed by approximately 3 weeks after the first administration of the ASO.

A sentinel dosing scheme is used, in which the first 2 participants of a cohort are dosed on the same day—1 subject that receives ASO, and 1 subject that receives placebo. If no clinically significant adverse events (AE) are observed after at least 24 hours of inpatient observation, the remaining participants in the same cohort are dosed. The remaining participants in the same cohort are dosed on the following day and are staggered with approximately 15 minutes separating each dosing. Pharmacokinetic, pharmacodynamic, and immunogenicity samples of the participants are collected at various timepoints.

Dose escalation does not occur until safety data through 3 days postdose is evaluated in at least 6 of 8 participants in Cohort 1, and at least 9 of 12 participants in Cohort 2 (fasted-fed cohort). Preliminary systemic PK are assessed in Cohort 1 and Cohort 2 (to the extent available) prior to Cohort 3 dosing, to ensure that exposure is not approaching the NOAEL.

The transition from Part 1 to Part 2 is not initiated until after evaluation of safety data through 3 days postdose, from the top dose group of Part 1 (Cohort 3, 1200 mg).

### Example 4—ASO Study, Part 2

In Part 2, multiple ascending oral doses ranging from about 120 mg to about 1200 mg are assessed in healthy volunteers. Each of the Cohorts 1 to 4 consists of 12 participants who receive once daily oral doses of either about 120 mg (Cohort 1), about 360 mg (Cohort 2), about 720 mg (Cohort 3), or about 1200 mg (Cohort 4) of ASO or a placebo over a 14-day period, in an ascending dose cohort manner—9 subjects that receive ASO, and 3 subjects that receive placebo. The participants may receive the dose in either a fasted or fed condition, based on an assessment of tolerability and PK from Part 1. Each of the dose levels are taken using the 120 mg capsule strength, including the starting dose of 120 mg. Part 2 preferably includes at least 6 female participants in total.

Participants are screened 28 days prior to dose administration. Cohorts 1-4 have an inpatient period from Day −2 to Day 18, and an outpatient follow-up visit on Day 28. The duration of the study for all participants is approximately 8 weeks, including the 4-week screening period and 2 weeks post-administration of the ASO.

A sentinel dosing scheme is used for each Cohort 1-4, in the same manner as described for Part 1. Pharmacokinetic, pharmacodynamic, and immunogenicity samples are collected at various timepoints. Biopsy samples obtained by flexible sigmoidoscopy for study intervention concentration determination and biomarker analysis including whole blood samples for RNA are collected.

Dose escalations do not occur until safety data through 14 days after the initial dose administration is evaluated in at least 9 of the 12 participants from the previous cohort Preliminary systemic PK is assessed from Cohort 1 (120 mg) prior to dosing Cohort 3 (720 mg), to ensure that exposure is not approaching the NOAEL. The doses can be given under semi-fasted, modified fasted, or fed conditions based on tolerability and PK information (if measurable) from Part 1 and optional Meal timing cohort (if conducted). If semi-fasted, participants are fast overnight for at least 10 hours and continue fasting until a standard meal is served at 1 to 2 hours postdose on Day 1 to Day 14. Participants are not allowed to drink water from 1 hour prior to dosing until 1 hour after dosing. In a modified fasted state, no food is consumed for approximately X hours before and approximately Y hours after study intervention administration on all days of dosing. The timing conditions, X and Y, can be determined based on the preliminary PK results of the optional Meal timing cohort. Doses can be taken with approximately 240 mL of non carbonated water. An additional 60 mL of water may be ingested for the doses>720 mg. If fed, participants can consume a standard meal 30 minutes before dosing on Day 1 to Day 14.

Additional Cohorts 5 and 6 may be enrolled to investigate the potential durability of IRF5 knockdown for the determination of future doses and treatment regimens. The dose levels of Cohorts 5 and 6 are within the defined escalation range (i.e. 120 mg to 1200 mg). The dose levels are preferably the same as those used in Part 3 to facilitate comparison of PK and PD between healthy participants and participants with UC. Tissue biopsy specimens are collected at time points later than in Cohorts 1-4 (i.e. later than 24 hours post last dose). Biopsy information for Cohort 5 is collected on Days 15/16 and Days 29/30, and for Cohort 6 on Days 15/16 and Days 43/44. Initiation of Cohorts 5 and 6 only occurs after an assessment of safety at the respective dose levels. The PD in Cohorts 1 to 4 may also be assessed before initiation of Cohorts 5 and 6.

The transition from Part 2 to Part 3 is not initiated until after evaluation of the safety data through at least 4 days post last dose (Day 18) from the top dose group of Part 2 (Cohort 4, 1200 mg). Dosing in female participants of child bearing potential with UC does not proceed until after the results of reproductive toxicology studies are available.

### Example 5—ASO Study, Part 3

In Part 3, the PD of ASO is studied in participants with moderately to severely active ulcerative colitis. Unlike healthy volunteers, participants with UC have active inflammation including both activated macrophages and dendritic cells, and thus a greater potential to demonstrate pharmacodynamic effects of the study intervention. The study of patients with UC also allows for assessment of downstream effects of ASO, where healthy participants are unlikely to show any changes.

Multiple oral doses are assessed in participants with moderately to severely active UC. A single dose level of about 720 mg is administered daily. The dose level may be lowered prior to the initiation of Part 3, depending on an assessment of Part 2. Cohort 1 consists of 12 participants who receive once daily doses of either ASO or a placebo over a period of 6 weeks—10 subjects that receive ASO, and 2 subjects that receive placebo. Cohort 2 follows Cohort 1, and consists of 12 participants who receive once daily doses of either ASO or a placebo over a period of 2 weeks—10 subjects that receive ASO, and 2 subjects that receive placebo. The protocol for Cohort 2 may be amended or the study concluded based on data from Cohort 1. Additional cohorts can be added to consist of participants who receive once daily doses of either ASO or a placebo over a period of 3, 4, 5, 7, or 8 weeks—some subjects that receive ASO, and some subjects that receive placebo. The daily doses administrated can be in the range of 120 to 122 mg, (e.g., 120, 360, 720, 800, 900, 1000, 1100, or 1200 mg.

Participants are screened for a period of up to 6 weeks prior to dose administration. During the screening period, predose biopsy samples are collected through a flexible sigmoidoscopy. In one embodiment, participants may also be assessed for the presence and removal of adenomatous polyps. In these situations, the predose disease severity is assessed using the Mayo Score for ulcerative colitis activity, based on the area that covers subsequent flexible sigmoidoscopy procedures. Cohort 1 participants undergo a flexible sigmoidoscopy, procedure for the collection of a tissue biopsy sample on Day 43/44, and for Cohort 2 on Day 15/16. The duration of study participation for all participants is approximately 18 weeks, including the 6-week screening period followed by 12 weeks after the first administration of study invention. The dosing regime can include 2 to 6 weeks of active treatment followed by drug holiday of 6 to 10 weeks.

### Example 6—ASO Study Evaluation

Safety evaluations are conducted on all participants that receive any administration of ASO or placebo. The safety and tolerability of ASO is monitored by physical examinations, electrocardiograms, cardiac telemetry/Holter monitoring, clinical laboratory tests, vital signs, concomitant medications, and adverse event (AE) reporting using the Medical Dictionary for Regulatory Activities (MedDRA) system.

During the period of administration of ASO and follow-up, pharmacokinetic, pharmacodynamic, immunogenicity samples, and stool samples are collected at various timepoints. Pharmacokinetic evaluation is conducted by assessment of plasma, urine, tissue, and stool samples for concentration of ASO or metabolites. Pharmacodynamic evaluation is conducted by assessment of mucosal biopsy samples (rectum and sigmoid colon) collected via flexible sigmoidoscopy in participants in Part 2 and Part 3 of the study. Concentrations of ASO in colon tissue by dose level are determined to assess the uptake of ASO and the effect of ASO on IRF5 RNA. If IRF5 knockdown is observed in tissue biopsy samples, preliminary PK/PD modeling may be done after Part 2 to identify the therapeutic target tissue concentration and to support the dose selection for Part 3. Inflammatory PD markers include C-reactive protein (CRP), fecal calprotectin, and fecal lactoferrin. Microbiome analysis may also be conducted on fecal samples to evaluate the association between inflammatory proteins, microbial activities and ASO and/or UC. The relationships between microbiome, metabolites, and biomarkers in other tissue samples may also be assessed.

Immunogenicity evaluations are conducted by assessment of anti-ASO antibodies in serum samples collected from all participants. Other immunogenicity analyses may be performed to further characterize the immune responses that are generated. Dose levels in Parts 1, 2, and 3 may be adjusted based on a review of the emerging safety, tolerability, and PK data.

Biomarker evaluation is conducted by assessment of biopsy samples and peripheral blood samples for biomarkers associated with IRF5 target engagement and downstream PD effects. Total RNA is isolated for measurement of levels of IRF5 mRNA and protein, as well as analysis of changes in downstream mRNA and protein expression patterns that are relevant to ASO treatment and/or UC. Differential gene expression analyses may include proteins associated with pro-inflammatory and anti-inflammatory effects and the recruitment and proliferation of cells associated with inflammation and repair. The biopsy samples collected are also used for tissue analysis (e.g., histology, immunohistochemistry).

Pharmacogenomic (DNA) evaluation may be conducted by collection of blood samples from consenting participants, to search for links of specific genes to disease or response to drug—e.g., the identification of genetic factors that may influence the PK, PD, safety, or clinical effects of ASO and to identify genetic factors associated with UC. Genetic (DNA) variation may be an important contributory factor in interindividual differences in drug response and associated clinical outcomes. Genetic factors may also serve as markers for disease susceptibility and prognosis, may identify population subgroups that respond differently to the study intervention, and may enable the development of safer, more effective, and ultimately individualized therapies.

Clinical evaluations are conducted in Part 3. Mayo Scores and partial Mayo Scores may be assessed, as developed from the criteria of Truelove and Witts for mild, moderate, and severe UC, and from the criteria of Baron et al. for grading endoscopic appearance. Truelove, S. C., et al., Cortisone in ulcerative colitis: final report on a therapeutic trial, Brit Med J. 2(2947):1041-1048 (1955); Baron, J. H. et al., Variation between observers in describing mucosal appearances in proctocolitis, Brit Med J. 1(5375):89-92 (1964). Participants may be given an Inflammatory Bowel Disease Questionnaire (IBDQ) that evaluates the disease-specific health-related quality of life. Irvine, E. J., et al., Quality of life: a valid and reliable measure of therapeutic efficacy in the treatment of inflammatory bowel disease, Gastroenterology 106(2):287-296 (1994).

While particular embodiments of the present disclosure have been illustrated and described, it would be obvious to those skilled in the art that various other changes and modifications can be made without departing from the spirit and scope of the present disclosure. It is therefore intended to cover in the appended claims all such changes and modifications that are within the scope of this disclosure.

| SEQUENCE LISTING | |
|---|---|
| <210> 1 | |
| <211> 2955 | |
| <212> DNA | |
| <213> Homo sapiens | |
| <400> 1 | |
| SEQ ID NO: 1 | |
| gcagaaagcg gaactgagcc cgcgtgttct gaggccaggg cagggctgga gcgttctgaa | 60 |
| cacctccccg tcccagcccc tgggccaggc aagggccggc cttacctctc ctgggttggt | 120 |
| ggcagcagag ctgggctctg agggaggcct gcaatgtgag acagtagcag ctcagaggcg | 180 |
| gcactaggca ggtgcaaccc caaaagaccc ctctgccatg aaccagtcca tcccagtggc | 240 |
| tcccacccca ccccgccgcg tgcggctgaa gccctggctg gtggcccagg tgaacagctg | 300 |
| ccagtaccca gggcttcaat gggtcaacgg ggaaaagaaa ttattctgca tcccctggag | 360 |
| gcatgccaca aggcatggtc ccagccagga cggagataac accatcttca aggcctgggc | 420 |
| caaggagaca gggaaataca ccgaaggcgt ggatgaagcc gatccggcca agtggaaggc | 430 |
| caacctgcgc tgtgccctta acaagagccg ggacttccgc ctcatctacg acgggccccg | 540 |
| ggacatgcca cctcagccct acaagatcta cgaggtctgc tccaatggcc ctgctcccac | 600 |
| agactcccag ccccctgagg attactcttt tggtgcagga gaggaggagg aagaagagga | 660 |
| agagctgcag aggatgttgc caagcctgag cctcacagag gatgtcaagt ggccgcccac | 720 |
| tctgcagccg cccactctgc ggccgcctac tctgcagccg cccactctgc agccgcccgt | 780 |
| ggtgctgggt ccccctgctc cagaccccag ccccctggct cctccccctg gcaaccctgc | 840 |
| tggcttcagg gagcttctct ctgaggtcct ggagcctggg cccctgcctg ccagcctgcc | 900 |
| ccctgcaggc gaacagctcc cgccagacct gctgatcagc ccccacatgc tgcctctgac | 960 |
| cgacctggag atcaagtttc agtaccgggg gcggccaccc cgggccctca ccatcagcaa | 1020 |
| cccccatggc tgccggctct tctacagcca gctggaggcc acccaggagc aggtggaact | 1080 |
| cttcggcccc ataagcctgg agcaagtgcg cttccccagc cctgaggaca tccccagtga | 1140 |
| caagcagcgc ttctacacga accagctgct ggatgtcctg gaccgcgggc tcatcctcca | 1200 |
| gctacagggc caggaccttt atgccatccg cctgtgtcag tgcaaggtgt tctggagcgg | 1260 |
| gccttgtgcc tcagcccatg actcatgccc caaccccatc cagcgggagg tcaagaccaa | 1320 |
| gcttttcagc ctggagcatt ttctcaatga gctcatcctg ttccaaaagg gccagaccaa | 1380 |
| caccccacca cccttcgaga tcttcttctg ctttggggaa gaatggcctg accgcaaacc | 1440 |
| ccgagagaag aagctcatta ctgtacaggt ggtgcctgta gcagctcgac tgctgctgga | 1500 |
| gatgttctca ggggagctat cttggtcagc tgatagtatc cggctacaga tctcaaaccc | 1560 |
| agacctcaaa gaccgcatgg tggagcaatt caaggagctc catcacatct ggcagtccca | 1620 |
| gcagcggttg cagcctgtgg cccaggcccc tcctggagca ggccttggtg ttggccaggg | 1680 |
| gccctggcct atgcacccag ctggcatgca ataacaaggc tgcagacggt gactggccct | 1740 |
| ggcctcctgg gtggcggtgc ggactgatgt ggagatgtga cagccccgat gagcacctgg | 1800 |
| ctggctgcag ggtcctacct ctgggtttcc tggaagtgga tttgggccaa gaaggagagg | 1860 |
| gagaaaggcc cgagcccctg ccttcccggg cctttctctc ctgggctgtc tctggtctgg | 1920 |
| tcagcctggc tctcgggaaa ttcagccatg agcagggaaa gaactctccc aaccctgggg | 1980 |
| cctagctgta taggaggaat tgcctaaggg tggcccactc ttgtgattgc cccatttcct | 2040 |
| ctggcaacaa aagccagagt gttgtgggcc aagtcccccc acagggcctc tgcagggcat | 2100 |
| ggccctgatt tccctggttt gagactcact tcctcatctc cctgtcctct gagataatat | 2160 |
| gagtgagcac ttaggtatca tatcagatgc tcaaggctgg cagctacccc cttcttgaga | 2220 |
| gtccaagaac ctggagcaga aataattttt atgtattttt ggattaatga atgttaaaaa | 2280 |
| cagactcagc tgtttctttc cttttactac taccagttgc tcccatgctg ctccaccagg | 2340 |
| ccctgtttcg gatgccaact ggcccactcc ccaagcactt gcccccagct tgcgaccatt | 2400 |
| ggcactggga gggcctggct tctgggctga tgggtcagtt gggccttcat aaacactcac | 2460 |
| ctggctggct ttgccttcca ggaggaagct ggctgaagca agggtgtgga attttaaatg | 2520 |
| tgtgcacagt ctggaaaact gtcagaatca gttttcccat aaaagggtgg gctagcattg | 2580 |
| cagctgcatt tgggaccatt caaatctgtc actctcttgt gtatattcct gtgctattaa | 2640 |
| atatatcagg gcagtgcatg taaatcatcc tgatatattt aatatattta ttatattgtc | 2700 |
| ccccgaggtg gggacagtga gtgagttctc ttagtccccc cagagctggt tgttaaagag | 2760 |
| cctggcacct acccgctctc acttcatctg tgtcatctct gcacactcca gcccactttc | 2820 |
| tgccttcagc cattgagtgg aagctgcccc aggcccttac caggtgcaga tgcccaatct | 2880 |
| tgacgcccag ccatcagaac tgtgagccaa ataaaccttt ttctgtacaa attacccaaa | 2940 |
| aaaaaaaaaa aaaaa | 2955 |
| <210> 2 | |
| <211> 12896 | |
| <212> DNA | |
| <213> Homo sapiens | |
| <400> 2 | |
| SEQ ID NO: 2 | |
| gagagccacc ctcgccaggg gtgtaggcag gcgagaggag ggcctggagc tgtgggtcgg | 60 |
| ccacactgcg ccctcatttg tgtgcagccc cggaggacca gagtggggaa gcaccccacc | 120 |
| ctctcccagg gcccaactga gcactgcagc gggaggtacg gggttgtcaa atgacagttt | 180 |
| tgccattcca gattgccaaa agagccagtg gccagtctag ggcaccgcgc cgtctggcat | 240 |
| ctccctggag gccctgggcc tggcccgagg ctcagcccgg atctgcagtt gccagctcag | 300 |
| tgcggggccc ggagtggatt cgcggggcgg ggcggggcac tgcccgcgcc cggagctcag | 360 |
| cagcagctgc ccaggggcgg gggcggcaag acgcggaagt gcccggcagg ttggcggacc | 420 |
| ggcgggaggc gcagcctggg cagagctcag cttggtcccg ccgcccggcc ggtgctccct | 480 |
| ggcgcagcca cgcaggcgca ccgcagacag gtgggtcccg gccgccgcgc tctcctctct | 540 |
| gcgtccgcgc ccggcgcgcc ccgagggtgg cgggagcggt gccggctact gcccccaagt | 600 |
| ctaggcctag actgggcccc gcgcccccca ggcacctgcg ggcggcggga tgaagactgg | 660 |
| agtagggcgg ggtccgcgtc cagctgcgcc tggaaagcga gctcgggggg gtgcctacag | 770 |
| cagggtgcgc ccggccggcc tgggacttcc aaagcgcctc ccacgccccg atcggtttgg | 780 |
| ggtgctggcg cccggggagc ccagtgaccc aggcggcgga gtgggcagcg ctgcgggggg | 840 |
| cgccggctct gctgctctcc ctccccctcg ccatcgccca gaatgggggt tcccgggagc | 900 |
| cccgctggag gctggcttgg accacagagg agcgaggccc gatccttact ttcgatgcac | 960 |
| tcgcccttgc tcttaccggg ccaccctcac cctttcggaa aagaggttga ggttaaagcg | 1020 |
| ttcatccccc gggatcttca ggccaatggc aggaactgtg caagagtctg ggggaagatg | 1080 |
| gtgtcaggta gaggctgcgt ccctgggctc gcggccggga atggcagact ctcgtccccc | 1140 |
| gagcagcgga aaaggatggg gcgcaatagt tcctgggctg gtttcctcag gtcccgtccc | 1200 |
| agaacttaag aaggcaacaa tgaagaggct aaacgtggag gaaaagtgag gctagcatgg | 1260 |
| ccgggatgcg tggggagatg gttgtctccg gaccccggga ggggcgggag cgggtacctg | 1320 |
| ggagcagagg ctggagtggg ggactttccc agcctcgcgg ccacgctgaa cacagcaggg | 1380 |
| cgaggaccgg ggtgctgctc ctccagcagc aggaggagag gtccagaggc cgactctgga | 1440 |
| ggtgggggtg gctccgcggg ctggcccagg gggtgtgccc cagcggagca cgcgggaggg | 1500 |
| gtgggggcgg aggggagggg ggagcagggg cggaggactg ggctgggcct gggctcctcg | 1560 |
| agggcccaga atggggataa gtgaccagaa ccagagaggg ctcggctgta tcgatgtagg | 1620 |
| aaactgtagc ccctcaggag gccctctggc aagacctccc cttcctgccc ccacccccag | 1680 |
| tagttatggg gcctggggtg ctggggctga ggggttccaa gagtcaaggg aagcactggg | 1740 |
| aaatcacccc tttttatcta aaggccctac tttggggttt ttcccctgta ccctggtctt | 1800 |
| cccctaccct gaccctggga ggaagctgaa agaagcttct ttctgggcac cttttgcccc | 1860 |
| agagcctcag cctgtctgga ccaggtgggc agcagggccc agggtgtggg cagctgaccc | 1920 |
| ggaggggtgg gatttggggg tcagggcctg tacagggaac cccttgtcct ctccctgagc | 1980 |
| tgggtgtggg tttgcaagga gacatgtgac ccacaccaac cctgggagca gcagggcgcc | 2040 |
| tgctgtctgg ccactcttac taggactgct gtggcacttc ctcccctagt gggtccctgg | 2100 |
| tgcccatgaa ttgcagctcc cgggtggtgg tgggggcact gtctcctggg actccagcat | 2160 |
| ggccctgggg tgagctgtgg gcttacccca cctcagcagg tcctctaggg ctgcccactg | 2220 |
| gatgcttcgc cgcctcscac aattgtaggg acttcctcag gctgttggat ttccccacct | 2280 |
| tccggggctc aggtccattg acttaggtct agggctccat acatttcacc cagagactcc | 2340 |
| ggagcctggc aggcagacct gttctgacac cgaacttcca aagtcatggg ccttgattgg | 2400 |
| ggtggtctga attagaccta gcccttttct gggcagaagg gagcttctag gaggatggat | 2460 |
| gctgttcggg ttagagctcg tgtggaccta gctgcaggca aaagccttga ggctgagtcc | 2520 |
| ctcctgtggc atggtggaca gactctcgct catcacagcc gggcttgtca cgggagctcc | 2580 |
| tcctccacac ccctccctaa gctgcctgta tggacgcggc cctctgacac tgaggtcgga | 2640 |
| gttatcattt caaaaccttg ctctgtatta aacagccgtg ttgggcaggg ccagactgct | 2700 |
| ggactgacag tagggggcag gcagccggac cctctgagct ccccaacggc accagcgcct | 2760 |
| gcacggcctc agcccagggg gtcatcaggg aagctctccc cgattctgtg cagacagagc | 2820 |
| ttcctctgtc cacccttgct cggccagaat tgtgtgccgc tggtgactgg cacccctcta | 2880 |
| ttctagggcc aaggcctctc aggggtctac agatacaact atgggtgggt gcacacccat | 2940 |
| gttataaacc acactaaatg cacaaaaact gtctgcaagt ttggggtgcg gggaacagct | 3000 |
| ctgggtggga ggttggaaat ttggtctggg ggacccactc ggctccctcc ctcagcccac | 3060 |
| agtgagtctg gtttctgagt tgtcccggtc tagccacttt cgtttcccct ggggccgggt | 3120 |
| ggaggctggg gcagaaagcg gaactgagcc cgcgtgttct gaggccaggg cagggctgga | 3180 |
| gcgctctgaa cacctccccg tcccagcccc tgggccaggc aagggccggc cttacctctc | 3240 |
| ctgggttggt ggcagcagag ctgggctctg agggaggcct acaatgtgag acagtagcag | 3300 |
| ctcagaggcg gcactaggca ggtgcaaccc caaaaggtac tgggcaggga atttcagggg | 3360 |
| aaactgaggc cccatgctgc aaggccaaag caggcccaga cacagggagt tcagctcgaa | 3420 |
| cgtttgctcc ttacttgctg accgacacat tcacttctga tgggctgtgc tgtgtaatga | 3480 |
| cccagtcctt tctgtcttcc acccaggggt ctcaagtgaa gggccaaggg catgggtaag | 3540 |
| gggaggagag ggacagaagg ggactcagga ctgtggagag tttccggttc tgggctggag | 3600 |
| agggagttgg gcaggtgaag gggaggaggc agggtccctc tggcctgact ccagggaggc | 3660 |
| agtgaagctg tggcctggga ggcggggctc gtgcccctgg gtaggtagca gttagagctg | 3720 |
| tggcttctgt ttcctgtagc cttcctaaca ggctccggca ggcgttaggg ccttcctaag | 3780 |
| tgccacccga atgcgtgtcc agtgggagga gaagggggag gaccaagaac ctagatgaat | 3840 |
| ggccctagag aggtaacatc cgtttggtgc ctgctgtcat tggaggtggg ttgagttggt | 3900 |
| tgatgggatt tctctaaaga tattgagaaa ctgctttcct ctgagggcag gtgtccttgg | 3960 |
| gagctccaga tgagattctt gttgagggca tctccggagg gaactctcag gggatggagg | 4020 |
| gtgagaaaac ttgggcaggg aagaatcgaa ctaggggtgt agactgagca ggaggctgtc | 4080 |
| ttcgggctga tcccacagag cgctcgctgt cccaggtagc cagcttgaga caagacagcc | 4140 |
| gggcttttgg tgtcaggcag tcactggctg tgggctgccc cccaagagca aggggtgggc | 4200 |
| ctaacctgtg gtgtgagtag tggaaggcgg ttctctggcc aacgggctgc ttgctgctgt | 4260 |
| tagcagttgg agaatggatg cctctgcccg gtaaagggca cctggggccg cgcccgcagc | 4320 |
| attcactaac tcgtaactct ccttcccttc ctccaaacac aaaattccat gacactagac | 4380 |
| aagaaagctg atgcttggaa aagagaattg gccttaaata cctagatgga ctggagagac | 4440 |
| catcctttgt ggtccatagc ttgacctctg agtaccctgt ccccatccac ctgcagacct | 4500 |
| gctgaggctc ccctctggct ttctcctgca gacccctctg ccatgaacca gtccatccca | 4560 |
| gtggctccca ccccaccccg ccgcgtgcgg ctgaagccct ggctggtggc ccaggtgaac | 4620 |
| agctgccagt acccagggct tcaatgggtc aacggggaaa agaaattatt ctgcatcccc | 4680 |
| tggaggcatg ccacaaggca tggtcccagc caggacggag ataacaccat cttcaaggta | 4740 |
| agccccgggg aggaggttgg ctggacctcc agggcaccct gtccccagaa gaggagcgca | 4800 |
| cataacgcac acaggcagct cctcgaggct ggccacccgc ccagctacca tgctgctgct | 4860 |
| gatgccgggc ccggactaag gggatgcaga cgtagacaca gggtacacct ttttcctttt | 4920 |
| tttttttttt taagacagag tctcgctctg tagcccaggc tggagtgcag tggcacgatc | 4980 |
| tcagctcact gcaacctctg cctcctgggt tcaagcaatt ctcctgcctc agcctcctga | 5040 |
| gtagctggga ttacaggcat gagccaccac gcctggccta gggcacatct tttctaacct | 5100 |
| gcaccctaga gcatcgtggg gactgagggt ccccagaagg ccttcccata actcgtccta | 5160 |
| ctcacccttt gctcgtctca ctcctattac tcatgaggac ttgttcagtg cacgcatatg | 5220 |
| ctaaaggaag ccaacgatca tcatccttct aaaaatttta tttttaaatt agtacatatt | 5280 |
| tatggggtac acagtggtga tttgataagc acagtgatca gatcaggtac ttagcatatc | 5340 |
| cataatctca aacatttgtc atttctttgt gttgggaaca aactattttt aatataagat | 5400 |
| tcagatacat catcaatctt tcaattgttt aaattttcta atttttttta gagacagggc | 5460 |
| ctcacactgt tgaccagatt ccaatttttt tttttttttt tttttttttt ttgagacagg | 5520 |
| gtctcactct gtcacccagg ctggagtgca gtggcttgat ctcagctcac tgcagcctcg | 5580 |
| acctcccagg ctcaggtgag tctcccatct caacctcctt gagtagctgg gattacaggt | 5640 |
| gcctgccacc acaactggct aattttttgt acttttagta gagactttgc catgttgccc | 5700 |
| aggctggtct tgaactcctg gactcaagca atccacccac ctcagcctcc cagagtgctg | 5760 |
| ggattacagg tgtgagccac catgcccggc ccaatctttt tttttaattg atgtactaca | 5820 |
| actgtacata catactctct tttttttttt ttgagatgag ttgttgctct gttgcctagt | 5880 |
| gttgcaccat tggtgagcag tggtgcagtc atagcacaac ctccaactca gctcaagtga | 5940 |
| tcctccagtc tcagcttcct gagtagccgt gactacaggt gcatgccacc atgcccagct | 6000 |
| aatttttttt cttttttttg agatggagtc ttgctttttc accctagctg gagtgcaatg | 6060 |
| gcacaatctc agctcactgc aacctctgcc acctgggttc aagcaattct cctgcctcag | 6120 |
| cctcccgagt agctaggatt acaggcacct gccaccatgc ccggctaatt tttttttttt | 6180 |
| tttttttttt tttttttttt tttgtatttt tggtaaagac agggtttcac cctgttggcc | 6240 |
| aggctggtct tgaactcctg acctcatgat ccacctgcct tggcctccca aagtgctggg | 6300 |
| attacaggcg tgagccaccg tgcccagcct aattttttga ttttgatatt tgtaaagatg | 6360 |
| aggtctcact ttgttgccca ggctggtctc aagctcatgg gctcaagtga tcctcccacc | 6420 |
| tcagcctcct gagtagctgg tatcacaggc gcaagccact gtgctctctc caataatctt | 6480 |
| aatgctagga tgtacctcgc ataatagttt ttgcttacat tttagttttt ttgcacatgt | 6540 |
| gtgtatatgg aatgcaaaat tggaatcaag tgaatatctt gatttatagc ctggattttt | 6600 |
| ttcacccaat atcaggatca gctttccttt ccaacatacg tccacatcac ttttcgtgac | 6660 |
| tgtagaattt ccactggaat ggtttagcac aatttggtca ctgagtcttg gtttttagat | 6720 |
| acttgagatt gcttttaaaa cctgaactta aaaaccacat tgtcatgtag gctgtcttaa | 6780 |
| tgcttccctt tttttcatcc tcaattattt tgggggttga tttcctagag ttgaaaattg | 6840 |
| ctgggtcaaa ggcaatgctt attttttaaa actttgggta catattttct tagtttttgg | 6900 |
| gcttgatcct tacctttcca aaattctttt gctaatccat ttattttgta taggtaatac | 6960 |
| agtcacaaaa ttcaacatta aaaggcatag aatggtttag agcaaaaagt ctccctcctt | 7020 |
| ccctggttca tcaccaccca ctcccctccc tagcctttcc ctagaggcag ccgtgttgtc | 7080 |
| attttcttgt ttagccttcc tgagagattt ttatgcacat gtaagcaaat atggaactat | 7140 |
| cctttcctct acttttcata agaaatgcta gatatctgca tatttgtcta gacttaatac | 7200 |
| ttcttgacga ttgctacatg tcagcttata aacagtttcc tgctttttct ttttttgtgc | 7260 |
| tgcccagtat ttttcattca atgggttggc cgtaatttca ccagcccctc attgatggat | 7320 |
| gttgcattgg gtattttggg tcatctttta cacacagcac tgctgcgaat aactttgtgt | 7380 |
| gtgcaatatt ttgtgtgaat gcttctatga tatttaggat gaattcctag aaatcaaata | 7440 |
| gctgggttaa aaggtagaaa gaaatgttgg taaccctcac ctcacctaat tgccattcaa | 7500 |
| aataatacca acagctccag tgtctcgagc ctgtcctatg tgcaggttta gctctgagtg | 7560 |
| ctttacggac atcaactccc ctcatctttt ttgagatagg gtatcactct gtcgtccagg | 7620 |
| ctggagtgca gagcataatc atggctcact gcagccttga actccccagc tcaagcaatc | 7680 |
| ctctcacctc agcctcctga gtagctggga ccacaggcac ccaccactat gcccagctaa | 7740 |
| ttttttgtaa agacaggagt ctcgttatgt tgcctaggct ggactccaac tcctgggctc | 7800 |
| aagcagtcct cccaccttgg cctcccaaaa tgttgggatt acaggtgcga gccactgtgc | 7860 |
| ccagccaaca tcaacccctt gagtcttcgc tgccactcca tgaggtgggc aactgtgagg | 7920 |
| agatcaagac aaagggaggc agtgacatgg cctggcacac agctggcggg gggcacttcc | 7980 |
| agattcaaac ccagcctggc tccagggtgc tcacccttat cccgcacgct gtcctcctta | 8040 |
| gagatcacac tgtttcaccc tcccaggagc agggactatg gatgcatctc atagccccta | 8100 |
| gatttcatgt agcttgctgg cagcctgagg gctggggcag gactgtggca gactcccacg | 8160 |
| ctgcaaacca ggtctggggc tcagcgaggc tcagcctgta gccgaagttc tccccacaca | 8220 |
| gtagagtctc ctatgggaca ggaggcagac tggggctgtg gcagggatga ggttctctgt | 8280 |
| ggtcggctat ttcttcctgc cccaggcctg ggccaaggag acagggaaat acaccgaagg | 8340 |
| cgtggatgaa gccgatccgg ccaagtggaa ggccaacctg cgctgtgccc ttaacaagag | 8400 |
| ccgggacttc cgcctcatct acgacgggcc ccgggacatg ccacctcagc cctacaagat | 8460 |
| ctacgaggtc tgctccaatg gccctgctcc cacaggtatc aggcctagcc ctctgtgggc | 8520 |
| cacctgggag gctgtgcaat gtcctggccc ccagccatga gctcttgggt gcaggcaggc | 8580 |
| caagggcccc tctagcaggc agtggtccag gaaacgatgc gggggctccc gctaggtcat | 8640 |
| gacacccagg gcttccagga gtggctggga tggctcactg gcatatcagg aatggcttgg | 8700 |
| cgtgcagtca gggacctggg tgcttcttcc ttaccattgc cctcgttttg gcttctggct | 8760 |
| ccagcctagg tctcatggcc catggagtcg gggaggtctt tcccaatcct ggtggctgtg | 8820 |
| ccctccacct cgccctgtgt tgggggcagc tttggggaag gcagaagctg cataggagct | 8880 |
| acaggcagcc tctcagggga tcttgcttct cctccgacat tgactccttt actgccctgc | 8940 |
| ttttctctcc ctgctgtgca gactcccagc cccctgagga ttactctttt ggtgcaggag | 9000 |
| aggaggagga agaagaggaa gaggtgagtg tgggttgagg aggcaggtgg agccctggac | 9060 |
| gagctccctg ctgcccccat cggccttagg tttccgcagc cccactccca tggagccccg | 9120 |
| tggccctctc aatagttctc cttgtttctt ctcctgggat tctgaacgat aggagcacag | 9180 |
| tccccacctg ctccttccca gggcattgtc attaccctgt gtgtgtgacc cacgcagcag | 9240 |
| ttggggcttg gtaggtctga ctccctgcag aaggcaaatg aggaaagtga ggcaaagggc | 9300 |
| ttttctgacc tgcctgggat ggacgagctg ggaccggagg cagggtcttg cctgagctaa | 9360 |
| actgaggcta ggggagttgc ctcatagttc tcgcctgtta tttccccagc cccaggtcag | 9420 |
| tggaataacc tgtcctcctt tctctcccat ctcttccctc ccttgctggt ggtgtccctt | 9480 |
| cagctgcaga ggatgttgcc aagcctgagc ctcacaggtg gggccgggag gtggtggttg | 9540 |
| ggggtctagt atacagagaa gctataggta ccataggtac ctggaagggg gctgatggga | 9600 |
| ggctagggtg gcccagggct gggaggaggt gtgcctggga ggcagttcgt ggaggtggca | 9660 |
| ctgacagccg tccacacgca ctctctgtag atgcagtgca gtctggcccc cacatgacac | 9720 |
| cctattcttt actcaaagag gatgtcaagt ggccgcccac tctgcagccg cccactctgc | 9780 |
| ggccgcctac tctgcagccg cccactctgc agccgcccgt ggtgctgggt ccccctgctc | 9840 |
| cagaccccag ccccctggct cctccccctg gcaaccctgc tggcttcagg gagcttctct | 9900 |
| ctgaggtcct ggagcctggg cccctgcctg ccagcctgcc ccctgcaggc gaacacctcc | 9960 |
| tgccagacct gctgatcagc ccccacatgc tgcctcgtaa ggacccatgg ctgggcacgg | 10020 |
| ggaagoagtg ctgggggatt ggggtaggat tggcaaggag ggtggagggt gctggactcc | 10080 |
| cttgggtggg aaaagtggga gggcggatgg ggctgggcct ggccactggg ctgcagaatg | 10140 |
| gggaggcgtg gggctcaagg acgggatggg cctgccttct gccccacagt gaccgacctg | 10200 |
| gagatcaagt ttcagtaccg ggggcggcca ccccgggccc tcaccatcag caacccccat | 10260 |
| ggctgccggc tcttctacag ccagctggag gccacccagg agcaggtgga actcttcggc | 10320 |
| cccataagcc tggagcaagt gcgcttcccc agccctgagg acatccccag tgacaagcag | 10380 |
| cgcttctaca cgaaccagct gctggatgtc ctggaccgcg ggctcatcct ccagctacag | 10440 |
| ggccaggacc tttatgccat ccgcctgtgt cagtgcaagg tgttctggag cgggccttgt | 10500 |
| gcctcagccc atgactcatg ccccaacccc atccagcggg aggtcaagac caagcttttc | 10560 |
| agcctggagc attttctcaa tggtgagggc ccaaagctgt gatcctcctg gctgcctctt | 10620 |
| gcccagggca cggttccagc ctctgactag ggaccttgat tttgatgcag agctcatcct | 10680 |
| gttccaaaag ggccagacca acaccccacc acccttcgag atcttcttct gctttcggga | 10740 |
| agaatggcct gaccgcaaac cccgagagaa gaagctcatt actgtacagg tacatctccc | 10800 |
| ctatcccaaa gtcggccttg gcttgaaaac tggggaatcc tggggctagg cccttgcccc | 10860 |
| aggctggagg ctcagggctc cctgagcagt gtgaacttgg cggccagaga ccatcaaggc | 10920 |
| tcagagccgg agaatgcggt ctattactca cccctgatgg ctgtcctcat gcacagctgg | 10980 |
| atctggcagc cctgccacag gtctccctgt ctcatctcct ctttgcctcc caggtggtgc | 11040 |
| ctgtagcagc tcgactgctg ctggagatgt tctcagggga gctatcttgg tcagctgata | 11100 |
| gtatccggct acagatctca aacccagacc tcaaagaccg catggtggag caattcaagg | 11160 |
| agctccatca catctggcag tcccagcagc ggttgcagcc tgtggcccag gcccctcctg | 11220 |
| gagcaggcct tggtgttggc caggggccct ggcctatgca cccagctggc atgcaataac | 11280 |
| aaggctgcag acggtgactg gccctggctt cctgggtggc ggtgcggact gatgtggaga | 11340 |
| tgtgacagcc ccgatgagca cctggctggc tgcagggtcc tacctctggg tttcctggaa | 11400 |
| gtggatttgg gccaagaagg agagggagaa aggcccgagc ccctgccttc ccgggccttt | 11460 |
| ctctcctggg ctgtctctgg tctggtcagc ctggctctcg ggaaattcag ccatgagcag | 11520 |
| ggaaagaact ctcccaaccc tggggcctag ctgtatagga ggaattgcct aagggtggcc | 11530 |
| cactcttgtg attgccccat ttcctctggc aacaaaagcc agagtgttgt gggccaagtc | 11640 |
| cccccacagg gcctctgcag ggcatggccc tgatttccct ggtttgagac tcacttcctc | 11700 |
| atctccctgt cctctgagat aatatgagtg agcacttagg tatcatatca gatgctcaag | 11760 |
| gctggcagct acccccttct tgagagtcca agaacctgga gcagaaataa tttttatgta | 11320 |
| tttttggatt aatgaatgtt aaaaacagac tcagctgttt ctttcctttt actactacca | 11880 |
| gttgctccca tgctgctcca ccaggccctg tttcggatgc caactggccc actccccaag | 11940 |
| cacttgcccc cagcttgcga ccattggcac tgggagggcc tggcttctgg gctgatgggt | 12000 |
| cagttgggcc ttcataaaca ctcacctggc tggctttgcc ttccaggagg aagctggctg | 12060 |
| aagcaagggt gtggaatttt aaatgtgtgc acagtctgga aaactgtcag aatcagtttt | 12120 |
| cccataaaag ggtgggctag cattgcagct gcatttggga ccattcaaat ctgtcactct | 12180 |
| cttgtgtata ttcctgtgct attaaatata tcagggcagt gcatgtaaat catcctgata | 12240 |
| tatttaatat atttattata ttgtcccccg aggtggggac agtgagtgag ttctcttagt | 12300 |
| ccccccagag ctggttgtta aagagcctgg cacctacccg ctctcacttc atctgtgtca | 12360 |
| tctctgcaca ctccagccca ctttctgcct tcagccattg agtggaagct gccccaggcc | 12420 |
| cttaccaggt gcagatgccc aatcttgatg cccagccatc agaactgtga gccaaataaa | 12480 |
| cctttttctg tataaattac ccagcctcgg gtcttcgttt acagcaacgc aaaatagatt | 12540 |
| aaacccccat aaatgttcaa ggataccttg ccccacagcc tcgtccacag aatatattgt | 12600 |
| cactgtttgg atttttgcca acctgacagg tgagatagta tctcagtgcc acttctcatt | 12660 |
| atcagcaagg ctgagtagct tttcacatgg ttaagtggcc tgtacagatt tttttaaata | 12720 |
| attttagaat ggttttagat ttatggaaaa gttcctaata gagttcctat ggacccacac | 12780 |
| tttctccaat tattaacatc ttacattact ataacacact tgtgacaata atgaaaccat | 12840 |
| gtggacaatt actatgaact caacttcttt atttggattt catgagtttt tcggat | 12896 |
| <400> 3 | |
| <210> 3 | |
| <211> 2910 | |
| <213> Homo sapiens | |
| <400> 3 | |
| SEQ ID NO: 3 | |
| agtgcccggc aggttggcgg accggcggga ggcgcagcct gggcagagct cagcttggtc | 60 |
| ccgccgcccg gccggtgctc cctggcgcag ccacgcaggc gcaccgcaga cagacccctc | 120 |
| tgccatgaac cagtccatcc cagtggctcc caccccaccc cgccgcgtgc ggctgaagcc | 180 |
| ctggctggtg gcccaggtga acagctgcca gtacccaggg cttcaatggg tcaacgggga | 240 |
| aaagaaatta ttctgcatcc cctggaggca tgccacaagg catggtccca gccaggacgg | 300 |
| agataacacc atcttcaagg cctgggccaa ggagacaggg aaatacaccg aaggcgtgga | 360 |
| tgaagccgat ccggccaagt ggaaggccaa cctgcgctgt gcccttaaca agagccggga | 420 |
| cttccgcctc atctacgacg ggccccggga catgccacct cagccctaca agatctacga | 480 |
| ggtctgctcc aatggccctg ctcccacaga ctcccagccc cctgaggatt actcttttgg | 540 |
| tgcaggagag gaggaggaag aagaggaaga gctgcagagg atgttgccaa gcctgagcct | 600 |
| cacagatgca gtgcagtctg gcccccacat gacaccctat tctttactca aagaggatgt | 660 |
| caagtggccg cccactctgc agccgcccac tctgcggccg cctactctgc agccgcccac | 720 |
| tctgcagccg cccgtggtgc tgggtccccc tgctccagac cccagccccc tggctcctcc | 780 |
| ccctggcaac cctgctggct tcagggagct tctctctgag gtcctggagc ctgggcccct | 840 |
| gcctgccagc ctgccccctg caggcgaaca gctcctgcca gacctgctga tcagccccca | 900 |
| catgctgcct ctgaccgacc tggagatcaa gtttcagtac cgggggcggc caccccgggc | 960 |
| cctcaccatc agcaaccccc atggctgccg gctcttctac agccagctgg aggccaccca | 1020 |
| ggagcaggtg gaactcttcg gccccataag cctggagcaa gtgcgcttcc ccagccctga | 1080 |
| ggacatcccc agtgacaagc agcgcttcta cacgaaccag ctgctggatg tcctggaccg | 1140 |
| cgggctcatc ctccagctac agggccagga cctttatgcc atccgcctgt gtcagtgcaa | 1200 |
| ggtgttctgg agcgggcctt gtgcctcagc ccatgactca tgccccaacc ccatccagcg | 1260 |
| ggaggtcaag accaagcttt tcagcctgga gcattttctc aatgagctca tcctgttcca | 1320 |
| aaagggccag accaacaccc caccaccctt cgagatcttc ttctgctttg gggaagaatg | 1380 |
| gcctgaccgc aaaccccgag agaagaagct cattactgta caggtggtgc ctgtagcagc | 1440 |
| tcgactgctg ctggagatgt tctcagggga gctatcttgg tcagctgata gtatccggct | 1500 |
| acagatctca aacccagacc tcaaagaccg catggtggag caattcaagg agctccatca | 1560 |
| catctggcag tcccagcagc ggttgcagcc tgtggcccag gcccctcctg gagcaggcct | 1620 |
| tggtgttggc caggggccct ggcctatgca cccagctggc atgcaataac aaggctgcag | 1680 |
| acggtgactg gccctggctt cctgggtggc ggtgcggact gatgtggaga tgtgacagcc | 1740 |
| ccgatgagca cctggctggc tgcagggtcc tacctctggg tttcctggaa gtggatttgg | 1800 |
| gccaagaagg agagggagaa aggcccgagc ccctgccttc ccgggccttt ctctcctggg | 1860 |
| ctgtctctgg tctggtcagc ctggctctcg ggaaattcag ccatgagcag ggaaagaact | 1920 |
| ctcccaaccc tggggcctag ctgtatagga ggaattgcct aagggtggcc cactcttgtg | 1980 |
| attgccccat ttcctctggc aacaaaagcc agagtgttgt gggccaagtc cccccacagg | 2040 |
| gcctctgcag ggcatggccc tgatttccct ggtttgagac tcacttcctc atctccctgt | 2100 |
| cctctgagat aatatgagtg agcacttagg tatcatatca gatgctcaag gctggcagct | 2160 |
| acccccttct tgagagtcca agaacctgga gcagaaataa tttttatgta tttttggatt | 2220 |
| aatgaatgtt aaaaacagac tcagctgttt ctttcctttt actactacca gttgctccca | 2280 |
| tgctgctcca ccaggccctg tttcggatgc caactggccc actccccaag cacttgcccc | 2340 |
| cagcttgcga ccattggcac tgggagggcc tggcttctgg gctgatgggt cagttgggcc | 2400 |
| ttcataaaca ctcacctggc tggctttgcc ttccaggagg aagctggctg aagcaagggt | 2460 |
| gtggaatttt aaatgtgtgc acagtctgga aaactgtcag aatcagtttt cccataaaag | 2520 |
| ggtgggctag cattgcagct gcatttggga ccattcaaat ctgtcactct cttgtgtata | 2580 |
| ttcctgtgct attaaatata tcagggcagt gcatgtaaat catcctgata tatrzaatat | 2640 |
| atttattata ttgtcccccg aggtggggac agtgagtgag ttctcttagt ccccccagag | 2700 |
| ctggttgtta aagagcctgg cacctacccg ctctcacttc atctgtgtca tctctgcaca | 2760 |
| ctccagccca ctttctgcct tcagccattg agtggaagct gccccaggcc cttaccaggt | 2820 |
| gcagatgccc aatcttgatg cccagccatc agaactgtga gccaaataaa cctttttctg | 2880 |
| tataaattac ccaaaaaaaa aaaaaaaaaa | 2910 |
| <210> 4 | |
| <211> 2187 | |
| <212> DNA | |
| <213> Homo sapiens | |
| <400> 4 | |
| SEQ ID NO: 4 | |
| gcggcgggag gcgcagcctg ggcagagctc agcttggtcc cgccgcccgg ccggtgctcc | 60 |
| ctggcgcagc cacgcaggcg caccgcagac agacccctct gccatgaacc agtccatccc | 120 |
| agtggccccc accccacccc gccgcgtgcg gctgaagccc tggctggtgg cccaggtgaa | 180 |
| cagctgccag tacccagggc ttcaatgggt caacggggaa aagaaattat tctgcatccc | 240 |
| ctggaggcat gccacaaggc atggtcccag ccaggacgga gataacacca tcttcaaggc | 300 |
| ctgggccaag gagacaggga aatacaccga aggcgtggat gaagccgatc cggccaagtg | 360 |
| gaaggccaac ctgcgctgtg cccttaacaa gagccgggac ttccgcctca tctacgacgg | 420 |
| gccccgggac atgccacctc agccctacaa gatctacgag gtctgctcca atggccctgc | 480 |
| tcccacagac tcccagcccc ctgaggatta ctcttttggt gcaggagagg aggaggaaga | 540 |
| agaggaagag ctgcagagga tgttgccaag cctgagcctc acagatgcag tgcagtctgg | 600 |
| cccccacatg acaccctatt ctttactcaa agaggatgtc aagtggccgc ccactctgca | 660 |
| gccgcccact ctgcagccgc ccgtggtgct gggtccccct gctccagacc ccagccccct | 720 |
| ggctcctccc cctggcaacc ctgctggctt cagggagctt ctctctgagg tcctggagcc | 780 |
| tgggcccctg cctgccagcc tgccccctgc aggcgaacag ctcctgccag acctgctgat | 840 |
| cagcccccac atgctgcctc tgaccgacct ggagatcaag tttcagtacc gggggcggcc | 900 |
| accccgggcc ctcaccatca gcaaccccca tggctgccgg ctcttctaca gccagctgga | 960 |
| ggccacccag gagcaggtgg aactcttcgg ccccataagc ctggagcaag tgcgcttccc | 1020 |
| cagccctgag gacatcccca gtgacaagca gcgcttctac acgaaccagc tgctggatgt | 1080 |
| cctggaccgc gggctcatcc tccagctaca gggccaggac ctttatgcca tccgcctgtg | 1140 |
| tcagtgcaag gtgttctgga gcgggccttg tgcctcagcc catgactcat gccccaaccc | 1200 |
| catccagcgq gaggtcaaga ccaagctttt cagcctggag cattttctca atgagctcat | 1260 |
| cctgttccaa aagggccaga ccaacacccc accacccttc gagatcttct tctgctttgg | 1320 |
| ggaagaatgg cctgaccgca aaccccgaga gaagaagctc attactgtac aggtggtgcc | 1380 |
| tgtagcagct cgactgctgc tggagatgtt ctcaggggag ctatcttggt cagctgatag | 1440 |
| tatccggcta cagatctcaa acccagacct caaagaccgc atggtggagc aattcaagga | 1500 |
| gctccatcac atctggcagt cccagcagcg gttgcagcct gtggcccagg cccctcctgg | 1560 |
| agcaggcctt ggtgttggcc aggggccctg gcctatgcac ccagctggca cgcaataaca | 1620 |
| aggctgcaga cggtgactgg ccctggcttc ctgggtggcg gtgcggactg atgtggagat | 1680 |
| gtgacagccc cgatgagcac ctggctggct gcagggtcct acctctgggt ttcctggaag | 1740 |
| tggatttggg ccaagaagga gagggagaaa ggcccgagcc cctgccttcc cgggcctttc | 1800 |
| tctcctgggc tgtctctggt ctggtcagcc tggctctcgg gaaattcagc catgagcagg | 1860 |
| gaaagaactc tcccaaccct ggggcctagc tgtataggag gaattgccta agggtqgccc | 1920 |
| actcttgtga ttgccccatt tcctctggca acaaaagcca gagtgttgtg ggccaagtcc | 1980 |
| ccccacaggg cctctgcagg gcatggccct gatttccctg gtttgagact cacttcctca | 2040 |
| tctccctgtc ctctgagata atatgagtga gcacttaggt atcatatcag atgctcaagg | 2100 |
| ctggcagcta cccccttctt gagagtccaa gaacctggag cagaaataat ttttatgtat | 2160 |
| ttttggatta ataaatgtta aaaacag | 2187 |
| <210> 5 | |
| <211> 556 | |
| <212> DNA | |
| <213> Homo sapiens | |
| <400> 5 | |
| SEQ ID NO: 5 | |
| gcagaaagcg gaactgagcc cgcgtgttct gaggccaggg cagggctgga gcgttctgaa | 60 |
| cacctccccg tcccagcccc tgggccaggc aagggccggc cttaactctc ctgggttggt | 120 |
| ggcagcagag ctgggctctg agggaggcct gcaatgtgag acagtagcag ctcagaggcg | 180 |
| gcactaggca ggtgcaaccc caaaagaccc ctctgccatg aaccagtcca tcccagtggc | 240 |
| tcccacccca ccccgccgcg tgcggctgaa gccctggctg gtggcccagg tgaacagctg | 300 |
| ccagtaccca gggcttcaat gggtcaacgg ggaaaagaaa ttattctgca tcccctggag | 360 |
| gcatgccaca aggcatggtc ccagccagga cggagataac accatcttca aggcctgggc | 420 |
| caaggagaca gggaaataca ccgaaggcgt ggatgaagcc gatccggcca agtggaaggc | 480 |
| caacctgcgc tgtgccctta acaagagccg ggacttccgc ctcatctacg acgggccccg | 540 |
| ggacatgcca cctcag | 556 |
| <210> 6 | |
| <211> 2862 | |
| <212> DNA | |
| <213> Homo sapiens | |
| <400> 6 | |
| SEQ ID NO: 6 | |
| agtgcccggc aggttggcgg accggcggga ggcgcagcct gggcagagct cagcttggtc | 60 |
| ccgccgcccg gccggtgctc cctggcgcag ccacgcaggc gcaccgcaga cagacccctc | 120 |
| tgccatgaac cagtccatcc cagtggctcc caccccaccc cgccgcgtgc ggctgaagcc | 180 |
| ctggctggtg gcccaggtga acagctgcca gtacccaggg cttcaatggg tcaacgggga | 240 |
| aaagaaatta ttctgcatcc cctggaggca tgccacaagg catggtccca gccaggacgg | 300 |
| agataacacc atcttcaagg cctgggccaa ggagacaggg aaatacaccg aaggcgtgga | 360 |
| tgaagccgat ccggccaagt ggaaggccaa cctgcgctgt gcccttaaca agagccggga | 420 |
| cttccgcctc atctacgaco ggccccggga catgccacct cagccctaca agatctacga | 480 |
| ggtctgctcc aatggccctg ctcccacaga ctcccagccc cctgaggatt actcttttgg | 540 |
| tgcaggagag gaggaggaag aagaggaaga gctgcagagg atgttgccaa gcctgagcct | 600 |
| cacagaggat gtcaagtggc cgcccactct gcagccgccc actctgcggc cgcccactct | 660 |
| gcagccgccc actctgcagc cgcccgtggt gctgggtccc cctgctccag accccagccc | 720 |
| cctggctcct ccccctggca accctgctgg cttcagggag cttctctctg aggtcctgga | 780 |
| gcctgggccc ctgcctgcca gcctgccccc tgcaggcgaa cagctcctgc cagacctgct | 840 |
| gatcagcccc cacatgctgc ctctgaccga cctggagatc aagtttcagt accgggggcg | 900 |
| gccaccccgg gccctcacca tcagcaaccc ccatggctgc cggctcttct acagccagct | 960 |
| ggaggccacc caggagcagg tggaactctt cggccccata agcctggagc aagtgcgctt | 1020 |
| ccccagccct gaggacatcc ccagtgacaa gcagcgcttc tacacgaacc agctgctgga | 1080 |
| tgtcctggac cgcgggctca tcctccagct acagggccag gacctttatg ccatccgcct | 1140 |
| gtgtcagtgc aaggtgttct ggagcgggcc ttgtgcctca gcccatgact catgccccaa | 1200 |
| ccccatccag cgggaggtca agaccaagct tttcagcctg gagcattttc tcaatgagct | 1260 |
| catcctgttc caaaagggcc agaccaacac cccaccaccc ttcgagatct tcttctgctt | 1320 |
| tggggaagaa tggcctgacc gcaaaccccg agagaagaag ctcattactg tacaggtggt | 1330 |
| gcctgtagca gctcgactgc tgctggagat gttctcaggg gagctatctt ggtcagctga | 1440 |
| tagtatccgg ctacagatct caaacccaga cctcaaagac cgcatggtgg agcaattcaa | 1500 |
| ggagctccat cacatctggc agtcccagca gcggttgcag cctgtggccc aggcccctcc | 1560 |
| tggagcaggc cttggtgttg gccaggggcc ctggcctatg cacccagctg gcatgcaata | 1620 |
| acaaggctgc agacggtgac tggccctggc ttcctgggtg gcggtgcgga ctgatgtgga | 1630 |
| gatgtgacag ccccgatgag cacctggctg gctgcagggt cctacctctg gatttcctgg | 1740 |
| aagtggattt gggccaagaa ggagagggag aaaggcccga gcccctgcct tcccgggcct | 1800 |
| ttctctcctg ggctgtctct ggtctggtca gcctggctct cgggaaattc agccatgagc | 1860 |
| agggaaagaa ctctcccaac cctggggcct agctgtatag gaggaattgc ctaagggtgg | 1920 |
| cccactcttg tgattgcccc atttcctctg gcaacaaaag ccagagtgtt gtgggccaag | 1980 |
| tccccccaca gggcctctgc agggcatggc cctgatttcc ctggtttgag actcacttcc | 2040 |
| tcacctccct gtcctctgag ataatatgag tgagcactta ggtatcatat cagatgctca | 2100 |
| aggctggcag ctaccccctt cttgagagtc caagaacctg gagcagaaat aatttttatg | 2160 |
| tatttttgga ttaatgaatg ttaaaaacag actcagctgt ttctttcctt ttactactac | 2220 |
| cagttgctcc catgctgctc caccaggccc tgtttcggat gccaactggc ccactcccca | 2280 |
| agcacttgcc cccagcttgc gaccattggc actgggaggg cctggcttct gggctgatgg | 2340 |
| gtcagttggg ccttcataaa cactcacctg gctggctttg ccttccagga ggaagctggc | 2400 |
| tgaagcaagg gtgtggaatt ttaaatgtgt gcacagtctg gaaaactgtc agaatcagtt | 2460 |
| ttcccataaa agggtgggct agcattgcag ctgcatttgg gaccattcaa atctgtcact | 2520 |
| ctcttgtgta tattcctgtg ctattaaata tatcagggca gtgcatgtaa atcatcctga | 2580 |
| tatatttaat atatttatta tattgtcccc cgaggtgggg acagtgagtg agttctctta | 2640 |
| gtccccccag agctggttgt taaagagcct ggcacctacc cgctctcact tcatctgtgt | 2700 |
| catctctgca cactccagcc cactttctgc cttcagccat tgagtggaag ctgccccagg | 2760 |
| cccttaccag gtgcagatgc ccaatcttga tgcccagcca tcagaactgt gagccaaata | 2820 |
| aacctttttc tgtataaatt acccaaaaaa aaaaaaaaaa aa | 2362 |
| <210> 7 | |
| <211> 2604 | |
| <212> DNA | |
| <213> Homo sapiens | |
| <400> 7 | |
| SEQ ID NO: 7 | |
| agtgcccggc aggttggcgg accggcggga ggcgcagcct gggcagagct cagcttggtc | 60 |
| ccgccgcccg gccggtgctc cctggcgcag ccacgcaggc gcaccgcaga cagacccctc | 120 |
| tgccatgaac cagtccatcc cagtggctcc caccccaccc cgccgcgtgc ggctgaagcc | 180 |
| ctggctggtg gcccaggtga acagctgcca gtacccaggg cttcaatggg tcaacgggga | 240 |
| aaagaaatta ttctgcatcc cctggaggca tgccacaagg cacggtccca gccaggacgg | 300 |
| agataacacc atcttcaagg cctgggccaa ggagacaggg aaatacaccg aaggcgtgga | 360 |
| tgaagccgat ccggccaagt ggaaggccaa cctgcgctgt gcccttaaca agagccggga | 420 |
| cttccgcctc atctacgacg ggccccggga catgccacct cagccctaca agatctacga | 480 |
| ggtctgctcc aatggccctg ctcccacaga ctcccagccc cctgaggatt actcttttgg | 540 |
| tgcaggagag gaggaggaag aagaggaaga gctgcagagg atgttgccaa gcctgagcct | 600 |
| cacagtgacc gacctggaga tcaagtttca gtaccggggg cggccacccc gggccctcac | 660 |
| catcagcaac ccccatggct gccggctctt ctacagccag ctggaggcca cccaggagca | 720 |
| ggtggaactc ttcggcccca taagcctgga gcaagtgcgc ttccccagcc ctgaggacat | 780 |
| ccccagtgac aagcagcgct tctacacgaa ccagctgctg gatgtcctgg accgcgggct | 840 |
| catcctccag ctacagggcc aggaccttta tgccatccgc ctgtgtcagt gcaaggtgtt | 900 |
| ctggagcggg ccttgtgcct cagcccatga ctcatgcccc aaccccatcc agcgggaggt | 960 |
| caagaccaag cttttcagcc tggagcattt ttccaatgag ctcatcctgt tccaaaaggg | 1020 |
| ccagaccaac accccaccac ccttcgagat cttcttctgc tttggggaag aatggcctga | 1080 |
| ccgcaaaccc cgagagaaga agctcattac tgtacaggtg gtgcctgtag cagctcgact | 1140 |
| gctgctggag atgttctcag gggagctatc ttgatcagct gatagtatcc ggctacagat | 1200 |
| ctcaaaccca gacctcaaag accgcatggt ggagcaattc aaggagctcc atcacatctg | 1260 |
| gcagtcccag cagcggttgc agcctgtggc ccaggcccct cctggagcag gccttggtgt | 1320 |
| tggccagggg ccctggccta tgcacccagc tggcatgcaa taacaaggct gcagacggtg | 1380 |
| actggccctg gcttcctggg tggcggtgcg gactgatgtg gagatgtgac agccccgatg | 1440 |
| agcacctggc tggctgcagg gtcctacctc tgggtttcct ggaagtggat ttgggccaag | 1500 |
| aaggagaggg agaaaggccc gagcccctgc cttcccgggc ctttctctcc tgggctgtct | 1560 |
| ctggtctggt cagcctggct ctcgggaaat tcagccatga gcagggaaag aactctccca | 1620 |
| accctggggc ctagctgtat aggaggaatt gcctaagggt ggcccactct tgtgattgcc | 1680 |
| ccatttcctc tggcaacaaa agccagagtg ttgtgggcca agtcccccca cagggcctct | 1740 |
| gcagggcatg gccctgattt ccctggtttg agactcactt cctcatctcc ctgtcctctg | 1800 |
| agataatatg agtgagcact taggtatcat atcagatgct caaggctggc agctaccccc | 1860 |
| ttcttgagag tccaagaacc tggagcagaa ataattttta tgtatttttg gattaatgaa | 1920 |
| tgttaaaaac agactcagct gtttctttcc ttttactact accagttgct cccatgctgc | 1980 |
| tccaccaggc cctgtttcgg atgccaactg gcccactccc caagcacttg cccccagctt | 2040 |
| gcgaccattg gcactgggag ggcctggctt ctgggctgat gggtcagttg ggccttcata | 2100 |
| aacactcacc tggctggctt tgccttccag gaggaagctg gctgaagcaa gggtgtggaa | 2160 |
| ttttaaatgt gtgcacagtc tggaaaactg tcagaatcag ttttcccata aaagggtggg | 2220 |
| ctagcattgc agctgcattt gggaccattc aaatctgtca ctctcttgtg tatattcctg | 2280 |
| tgctattaaa tatatcaggg cagtgcatgt aaatcatcct gatatattta atatatttat | 2340 |
| tatattgtcc cccgaggtgg ggacagtgag tgagttctct tagtcccccc agagctggtt | 2400 |
| gttaaagagc ctggcaccta cccgctctca cttcatctgt gtcatctctg cacactccag | 2460 |
| cccactttct gccttcagcc attgagtgga agctgcccca ggcccttacc aggtgcagat | 2520 |
| gcccaatctt gatgcccagc catcagaact gtgagccaaa taaacctttt tctgtataaa | 2580 |
| ttacccaaaa aaaaaaaaaa aaaa | 2604 |
| <210> 8 | |
| <211> 2778 | |
| <212> DNA | |
| <213> Homo sapiens | |
| <400> 8 | |
| SEQ ID NO: 8 | |
| gtccagctgc gcctggaaag cgagctcgga cccctctgcc atgaaccagt ccatcccagt | 60 |
| ggctcccacc ccaccccgcc gcgtgcggct gaagccctgg ctggtggccc aggtgaacag | 120 |
| ctgccagtac ccagggcttc aatgggtcaa cggggaaaag aaattattct gcatcccctg | 180 |
| gaggcatgcc acaaggcatg gtcccagcca ggacggagat aacaccatct tcaaggcctg | 240 |
| ggccaaggag acagggaaat acaccgaagg cgtggatgaa gccgatccgg ccaagtggaa | 300 |
| ggccaacctg cgctgtgccc ttaacaagag ccgggacttc cgcctcatct acgacgggcc | 360 |
| ccgggacatg ccacctcagc cctacaagat ctacgaggtc tgctccaatg gccctgctcc | 420 |
| cacagactcc cagccccctg aggattactc ttttggtgca ggagaggagg aagaagaaga | 480 |
| ggaagagctg cagaggatgt tgccaagcct gagcctcaca gaggatgtca agtggccgcc | 540 |
| cactctgcag ccgcccactc tgcggccgcc tactctgcag ccgcccactc tgcagccgcc | 600 |
| cgtggtgctg ggtccccctg ctccagaccc cagccccctg gctcctcccc ctggcaaccc | 660 |
| tgctggcttc agggagcttc tctctgaggt cctggagcct gggcccctgc ctgccagcct | 720 |
| gccccctgca ggcgaacagc tcctgccaga cctgctgatc agcccccaca tgctgcctct | 780 |
| gaccgacctg gagatcaagt ttcagtaccg ggggcggcca ccccgggccc tcaccatcag | 840 |
| caacccccat ggctgccggc tcttctacag ccagctggag gccacccagg agcaggtgga | 900 |
| actcttcggc cccataagcc tggagcaagt gcgcttcccc agccctgagg acatccccag | 960 |
| tgacaagcag cgcttctaca cgaaccagct gctggatgtc ctggaccgcg ggctcatcct | 1020 |
| ccagctacag ggccaggacc tttatgccat ccgcctgtgt cagtgcaagg tgttctggag | 1080 |
| cgggccttgt gcctcagccc atgactcatg ccccaacccc atccagcggg aggtcaagac | 1140 |
| caagcttttc agcctggagc attttctcaa tgagctcatc ctgttccaaa agggccagac | 1200 |
| caacacccca ccacccttcg agatcttctt ctgctttggg gaagaatggc ctgaccgcaa | 1260 |
| accccgagag aagaagctca ttactgtaca ggtggtgcct gtagcagctc gactgctgct | 1320 |
| ggagatgttc tcaggggagc tatcttggtc agctgatagt atccggctac agatctcaaa | 1380 |
| cccagacctc aaagaccgca tggtggagca atccaaggag ctccatcaca tctggcagtc | 1440 |
| ccagcagcgg ttgcagcctg tggcccaggc ccctcctgga gcaggccttg gtgttggcca | 1500 |
| ggggccctgg cctatgcacc cagctggcat gcaataacaa ggctgcagac ggtgactggc | 1560 |
| cctggcttcc tgggtggcgg tgcggactga tgtggagatg tgacagcccc gatgagcacc | 1620 |
| tggctggctg cagggtccta cctctgggtt tcctggaagt ggatttgggc caagaaggag | 1680 |
| agggagaaag gcccgagccc ctgccttccc gggcctttct ctcctgggct gtctctggtc | 1740 |
| tggtcagcct ggctctcggg aaattcagcc atgagcaggg aaagaactct cccaaccctg | 1800 |
| gggcctagct gtataggagg aattgcctaa gggtggccca ctcttgtgat tgccccattt | 1860 |
| cctctggcaa caaaagccag agtgttgtgg gccaagtccc cccacagggc ctctgcaggg | 1920 |
| catggccctg atttccctgg tttgagactc acttcctcat ctccctgccc tctgagataa | 1980 |
| tatgagtgag cacttaggta tcatatcaga tgctcaaggc tggcagctac ccccttcttg | 2040 |
| agagtccaag aacctggagc agaaataatt tttatgtatt tttggattaa tgaatgttaa | 2100 |
| aaacagactc agctgtttct ttccttttac tactaccagt tgctcccatg ctgctccacc | 2160 |
| aggccctgtt tcggatgcca actggcccac tccccaagca cttgccccca gcttgcgacc | 2220 |
| attggcactg ggagggcctg gcttctgggc tgatgggtca gttgggcctt cataaacact | 2280 |
| cacctggctg gctttgcctt ccaggaggaa gctggctgaa gcaagggtgt ggaattttaa | 2340 |
| atgtgtgcac agtctggaaa actgtcagaa tcagttttcc cataaaaggg tgggctagca | 2400 |
| ttgcagctgc atttgggacc attcaaatct gtcactctct tgtgtatatt cctgtgctat | 2460 |
| taaatatatc agggcagtgc atgtaaatca tcctgatata tttaatatat ttattatatt | 2520 |
| gtcccccgag gtggggacag tgagtgagtt ctcttagtcc ccccagagct ggttgttaaa | 2580 |
| gagcotggca cctacccgct ctcacttcat ctgtgtcatc tctgcacact ccagcccact | 2640 |
| ttctgccttc agccattgag tggaagctgc cccaggccct taccaggtgc agatgcccaa | 2700 |
| tcttgatgcc cagccatcag aactgtgagc caaataaacc tttttctgta taaattaccc | 2760 |
| aaaaaaaaaa aaaaaaaa | 2778 |
| <210> 9 | |
| <211> 18000 | |
| <212> DNA | |
| <213> Homo sapiens | |
| <400> 9 | |
| SEQ ID NO: 9 | |
| ccctcctccc ccctgctccc caccccagcc accccactct acactcgctc acacagcgta | 60 |
| gcttctactc gagtcagggg agggtgcctc ctaccacacc ctcagtctgt gatattccag | 120 |
| ccaggggaaa tgggggaagg ggccatctca ctgtggggag tggggggccc ggacagacct | 180 |
| ccctgtcccc accgcctcct ggcccatcaa ggggaaatgg gagtgctcac acaagggccg | 240 |
| ggtgtggctt cacttcccct ctacttgccc ttccaccccc aggatggcac tgggaggcca | 300 |
| agggatccca gatcctggaa acactggagt gctgctctct gtccttcctt tctcctgggc | 360 |
| catgtctctg gccccagatg attcttggct caggttcttt cagttgaggg aagtcaggat | 420 |
| ctgagccaag ctgagcccct gcatagcctc tatcccggga gccccatatc aatgctgccg | 480 |
| gcttggtaac atcatccccc ttttagagac gaggaaatga gstttgaggt cctacttaag | 540 |
| gccacttggc tggaatacag caaagccagg tgcaagcccc ttctctgcac gcagcccgtg | 600 |
| ctgcttcttg aggttcagag gctctaagga aggggcctca cctgagcaag atggacagtg | 660 |
| ggaagggggc agagggcaga attctagggt ctcacggtcc ctcagaatgg gtgaggagta | 720 |
| tttgataaca aaagaaggct cttcttagct attgctccag atacgaccag catagaatat | 780 |
| ttttctatta agcataactg acctccagtg aaaaaggttc ccattttgtg gggagccact | 840 |
| tttgaaatag caatctcatt tcattattac tattttgaga taagatctca cttctacgcc | 900 |
| caggctggag tgcggtggtg tgcctcctgg gttcaagcaa ttctcccacc tcagcctccc | 960 |
| aagtaggtgg aactataggt gtgtgtcacc acgccaggct aatttttgta ttttttgtag | 1020 |
| agacaaggtt ttgccatatt gcccaggctg gtctcgaact cttggactca agtgatctgc | 1080 |
| ctgccttagc ctctcaaagt gttgggatta caaatgtgag ccaccgtgcc tggcctagca | 1140 |
| atttcatttt taaagaaaga aatataaaac gatctaccca ttcacatttt ccccatccat | 1200 |
| actctaaatt ttaaaaatat gaaataatga aatactcagc agatacaaat aataaaatac | 1260 |
| ttacatataa tgatatttcg acttcatatt agggcttcaa aggtcaggaa agagggagag | 1320 |
| atacagaaga gaaaaaaaaa aaacgttaac acatgagtgt ctttttttaa cttgttaaag | 1380 |
| gtaaagttca acccaggaga agggagggga gggaaggtca aagttctgat ccatcaacaa | 1440 |
| atactacttt atttgttaaa aatacaaggc tctgcttggc gcagtggctc ccgcctgtaa | 1500 |
| tcccagcact ttgggaggcc aaggcgggtg gatcgcctga ggtcaggagc tcgagaccag | 1560 |
| cctggccaac atggtgaaac cccgtctcta cttgggaggc tgaggcagga gaatcgcttg | 1620 |
| aactggggag gcagaggttg cagtgagctg agatcgcact gctgcactcc agcccgggtg | 1680 |
| acagagcaag actccatatc aaaaaataaa ataaaacaaa atgaacaatg aaaagacagg | 1740 |
| agggaggttc ctgagtaagc tgattcgagt ggaaatcaga catcaaaatt gaaacccgct | 1800 |
| gaattttcca aaaagccaga tgctcataga actgaagctt gagacactta catcagtaca | 1860 |
| cctgctgcct gttgaccaat tcctcttcct tgtccctcct gttttccttc cctgctatat | 1920 |
| aagcccctaa ctttagtcaa ggggagggat ggattggagg tttgtctccc ctctcaacag | 1980 |
| ctcactggct gatgtcaccg gaataaagtc ttccctggca atactcgctg tctcagtgat | 2040 |
| aggctttctg tgcggtgagc agccagacct ggaccaaacc cctggcaatc cataacaaaa | 2100 |
| tcataagcaa aatcaaccag gaagctattt gcaccctgga accccacctg ggaggggaag | 2160 |
| tcaaggcaga ctttccagag aaggcgatgc tgaaaatttc agaaacagaa attttgaaga | 2220 |
| gcaagagtta ccaagcgaag aacattccat gagaaggaac aggaggtgtg tgaaggtgga | 2280 |
| ggttctgggg tgaggttctt tatggaatcg aaaacggttc agaaccacag gtcgacggtc | 2340 |
| aaggtagtag tgagaggagg tgggacaggc gaccacgccg ctgcccctgg gatgactgga | 2400 |
| aggcgactta ggggagctgg ggcgagacag gtgcagggtt tgaggatgag aaaggcacag | 2460 |
| agtgactaga ggattcccgc ctgcaagcac atctggaagg ggtgtctgga tcctgggggc | 2520 |
| agcgactgtg ttctagggcg agagccaccc tcgccagggg tgtaggcagg cgagaggagg | 2580 |
| gcctggagct gtgggtcggc cacactgcgc cctcatttgt gtgcagcccc ggaggaccag | 2640 |
| agtggggaag caccccaccc tctcccaggg cccaactgag cactgcagcg ggaggtacgg | 2700 |
| ggttgtcaaa tgacagtttt gccattccag attgccaaaa gagccagtgg ccagtctagg | 2760 |
| gcaccgcgcc gtctggcatc tccctggagg ccctgggcct ggcccgaggc tcagcccgga | 2820 |
| tctgcagttg ccaggtcagt gcggggcccg gagtggattc gcggggcggg gcggggcact | 2880 |
| gcccgcgccc ggagctcagc agcagctgcc caggggcggg ggcggcaaga cgcggaagtg | 2940 |
| cccggcaggt tggcggaccg gcgggaggcg cagcctgggc agagctcagc ttggtcccgc | 3000 |
| cgcccggccg gtgctccctg gcgcagccac gcaggcgcac cgcagacagg tgggtcccgg | 3060 |
| ccgccgcgct ctcctctctg cgtccgcgcc cggcgcgccc cgagggtggc gggagcggtg | 312 |
| ccggctactg cccccaagtc taggcctaga ctgggccccg cgccccccag gcacctgcgg | 3180 |
| gcggcgggat gaagactgga gtagggcggg gtccgcgtcc agctgcgcct ggaaagcgag | 3240 |
| ctcgggtggg tgcctacagc agggtgcgcc cggccggcct gggacttcca aagcgcctcc | 3300 |
| cacgccccga tcggtttggg gtgctggcgc ccggggagcc cagtgaccca ggcggcggag | 3360 |
| tgggcagcgc tgcggggggc gccggctctg ctgctctccc tccccctcgc catcgcccag | 3420 |
| aatgggggtt cccgggagcc ccgctggagg ctggcttgga ccacagagga gcgaggcccg | 3480 |
| atccttactt tcgatgcact cgcccttgct cttaccgggc caccctcacc ctttcggaaa | 3340 |
| agaggttgag gttaaagcgt tcatcccccg ggatcttcag gccaatggca ggaactgtgc | 3600 |
| aagagtttgg gggaagatgg tgtcaggtag aggctgcgtc cctgggctcg cggccgggaa | 3660 |
| tggcagactc tcgtcccccg agcagcggaa aaggatgggg cgcaatagtt cctgggctgg | 3720 |
| tttcctcagg tcctgtccca gaacttaaga aggcaacaat gaagaggcta aacgtggagg | 3780 |
| aaaagtgagg ctagc&tggc cgggatgcgt ggggagatgg ttgtctccgg accccgggag | 3840 |
| gggcgggagc gggtacctgc gagcagaggc tggagtgggg gactttccca gcctcgcggc | 3900 |
| cacgctgaac acagcagggc gaggaccggg gtgctgctcc tccagcagca ggaggagagg | 3960 |
| tccagaggcc gactctggag gtgggggtgg ctccgcgggc tggcccaggg ggtgtgcccc | 4020 |
| agcggagcac gcgggagggg tgggggcgga ggggaggggg gagcaggggc ggaggactgg | 4080 |
| gctgggcctg ggctcctcga gggcccagaa tggggataag tgaccagaac cagagagggc | 4140 |
| tcggctgtat cgatgtagga aactgtagcc cctcaggagg ccctctggca agacctcccc | 4200 |
| ttcctgcccc cacccccagt agttatgggg cctggggtgc tggggctgag gggttccaag | 4260 |
| agtcaaggga agcactggga aatcacccct ttttatctaa aggccctact ttggggLLct | 4320 |
| tcccctgtac cctggtcttc ccctaccctg accctgggag gaagctgaaa gaagcttctt | 4380 |
| tctgggcacc ttttgcccca gagcctcagc ctgtctggac caggtqggca gcagggccca | 4440 |
| gggtgtgggc agctgacccg gaggggtggg atttgggggt cagggcctgt acagggaacc | 4500 |
| ccttgtcctc tccctgagct gggtgtgggt ttgcaaggag acatgtgacc cagaccsacc | 4560 |
| ctgggagcag cagggcgcct gctgtctggc cactcttact aggactgctg tggcacttcc | 4620 |
| tcccctagtg ggtccctggt gcccatgaat tgcagctcct gggtggtggt gggggcactg | 4680 |
| tctcctggga ctccagcatg gccctggggt gagctgtggg cttaccccac ctcagcaggt | 4740 |
| cctctagggc tgcccactgg atgcttcgct gcctcacaca attgtaggga cttcctcagg | 4800 |
| ctgttggatt tccccacctt ccggggctca ggtccattga cttaggtcta gggctccata | 4860 |
| catttcaccc agagactccg gagcctggca ggcagacctg ttctgacacc gaacttccaa | 4920 |
| agtcatgggc cttgattggg gtggtctgaa ttagacctag cccttttctg ggcagaaggg | 4930 |
| agcttctagg aggatggatg ctgttcgggt tagagctcgt gtggacctag ctgcaggcaa | 5040 |
| aagccttgag gctgagtccc ttctgtggca tggtggacag actctcgctc atcacagccg | 5100 |
| ggcttgtcac gggagctcct cctccacacc cctccctaag ctgcctgtat ggacgcggcc | 5160 |
| ctctgacact gaggtcggag ttatcatttc aaaaccttgc tctgtattaa acagccgtgt | 5220 |
| tgggcagggc cagactgctg gactgacagt agggggcagg cagccggacc ctctgagctc | 5230 |
| cccaacggca ccagcgcctg cacggcctca gcccaggggg tcattaggga aqccctcccc | 5340 |
| gattctgtgc agacagagct tcctctgtcc acccttgctc ggccagaatt gtgtgccgct | 5400 |
| ggtgactggc acccctctat tctagggcca aggcctctca ggggtctaca gatacaacta | 5460 |
| tgggtgggtg cacacccatg ttataaacca cactaaatgc acaaaaactg tctgcaagtt | 5520 |
| tggggtgcgg ggaacagctc tgggtgggag gttggaaatt tggtctgggg gacccactcg | 5580 |
| gctccctccc tcagcccaca gtgagtctgg tttctgagtt gtcccggtct agccactttc | 5640 |
| gtttcccctg gggccgggtg gaggctgggg cagaaagcgg aactgagccc gcgtgttctg | 5700 |
| aggccagggc agggctggag cgttctgaac acctccccgt cccagcccct gggccaggca | 5760 |
| agggccggcc ttacctctcc tgggttggtg gcagcagagc tgggctctga gggaggcctg | 5820 |
| caatgtgaga cagtagcagc tcagaggcgg cactaggcag gtgcaacccc aaaaggtact | 5880 |
| gggcagggaa tttcagggga aactgaggct ccatgctgca aggccaaagc aggcccagac | 5940 |
| acagggagtt cagctcgaac gtttgctcct tacttgctga ccgacacatt cacttctgat | 6000 |
| gggctgtgct gtgtaatgac ccagtccttt ctgtcttcca cccaggggtc tcaagtgaag | 6060 |
| ggccaagggc atgggtaagg ggaggagagg gacagaaggg gactcaggac tgtggagagt | 6120 |
| ttccggttct gggctggaga gggagttggg caggtgaagg ggaggaggca gggtccctct | 6180 |
| ggcctgactc cagggaggca gtgaagctgt ggcctgggag gcggggctcg tgcccctggg | 6240 |
| taggtagcag ttagagctgt ggcttctgtt tcctgtagcc ttcctaacag gctccggcag | 6300 |
| gcgttagggc cttcctaagt gctacccgaa tgcgtgtcca gtgggaggag aagggggagg | 6360 |
| accaagaacc tagatgaatg gccctagaga ggtaacatct gtttggtgcc tgctgtcatt | 6420 |
| ggaggtgggt tgagttggtt gatgggattt ctctaaagat attgagaaac tgctttcctc | 6480 |
| tgagggcagg tgtccttggg agctccagat gagattcttg ttgagggcat ctccggaggg | 6540 |
| aactctcagg ggatggaggg tgagaaaact tgggcaggga agaatcgaac taggggtgta | 6600 |
| gactgagcag gaggctgtct tcgggctgat cccacagagc gctcgctgtc ccaggtagcc | 6660 |
| agcttgagac aagacagccg ggcttttggt gtcaggcagt cactggctgt gggctgcccc | 6720 |
| ccaagagcaa ggggtgggcc taacctgtgg tgtgagtagt ggaaggcggt tctctggcca | 6780 |
| acgggctgct tgctgctgtt agcagttgga gaatggatgc ctctgcccgg taaagggcac | 6840 |
| ctggggccgc gcccgcagca ttcactaact cgtaactctc cttcccttcc tccaaacaca | 6900 |
| aaattccatg acactagaca agaaagctga tgcttggaaa agagaattgg ccttaaatac | 6960 |
| ctagatggac tggagagacc atcctttgtg gtccatagct tgacctctga gtaccctgtc | 7020 |
| cccatccacc tgcagacctg ctgaggctcc cctctggctt tctcctgcag acccctctgc | 7080 |
| catgaaccag tccatcccag tggctcccac cccaccccgc cgcgtgcggc tgaagccctg | 7140 |
| gctggtggcc caggtgaaca gctgccagta cccagggctt caatgggtca acggggaaaa | 7200 |
| gaaattattc tgcatcccct ggaggcatgc ggaggcatgc ggtcccagcc aggacggaga | 7260 |
| taacaccatc ttcaaggcaa gccccgggga ggaggttggc tggacctcca gggcaccctg | 7320 |
| tccccagaag aggagcgcac ataacgcaca caggcagctc ctcgaggctg gccacccgcc | 7380 |
| cagctaccat gctgctgctg atgccgggcc cggactaagg ggatgcagac gtagacacag | 7440 |
| ggtacacctt tttccttttt tttttttttt aagacagagt ctcgctctgt agcccaggct | 7500 |
| ggagtgcagt ggcacgatct cagctcactg caacctctgc ctcctgggtt caagcaattc | 7560 |
| tcctgcctca gcctcctgag tagctgggat tacaggcatg agccaccacg cctggcctag | 7620 |
| ggcacatctt ttctaacctg caccctagag catcgtgggg actgagggtc cccagaaggc | 7680 |
| cttcccataa ctcgtcctac tcaccctttg ctcgtctcac tcctattact catgaggact | 7740 |
| tgttcagtgc acgcatatgc taaaggaagc caacgatcat catctttcta aaaattttat | 7800 |
| ttttaaacta gtatatattt atggggtaca cagtggtgat ttgataagca cagtgatcag | 7860 |
| atcaggtact tagcatatcc ataatctcaa acatttgtca tttctttgtg ttgggaacaa | 7920 |
| actattttta atataagatt cagatacatc atcaatcttt caattgttta aattttctaa | 7980 |
| ttttttttag agacagggcc tcacactgtt gaccagattc caattttttt tttttttttt | 8040 |
| tttttttttt tgagacaggg tctcactctg tcacccaggc tggagtgcag tggcttgatc | 8100 |
| tcagctcact gcagcctcga cctcccaggc tcaggtgagt ctcccatctc aacctccttg | 8160 |
| agtagctggg attacaggtg cctgccacca caactggcta attttttgta cttttagtag | 8220 |
| agactttgcc atgttgccca ggctggtctt gaactcctgg actcaagcaa tccacccacc | 8280 |
| tcagcctccc agagtgctgg gattacaggt gtgagccacc atgcccggcc caatcttttt | 8340 |
| ttttaattga tgtactacaa ctgtacatat atactctttt tttttttttt tgagatgagt | 8400 |
| tgttgctctg ttgcctagtg ttgcaccatt ggtgagcagt ggtgcagtca tagcacaacc | 8460 |
| tccaactcag ctcaagtgat cctccagtct cagcttcctg agtagccgtg actacaggtg | 8520 |
| catgccacca tgcccagcta attttttttt ttttttttga gatggagtct tgctttttca | 8580 |
| ccctagctgg agtgcaatgg cacaatctca gctcactgca acctctgcca cctgggttca | 8640 |
| agcaattctc ctgcctcagc ctcccgagta gctaggatta caggcacctg ccaccatgcc | 8700 |
| cggctaattt tttttttttt tttttttttt tttttttttt ttgtattttt ggtaaagaca | 8760 |
| gggtttcacc ctgttggcca ggctggtctt gaactcctga cctcatgatc cacctgcctt | 8820 |
| ggcctcccaa agtgctggga ttacaggcgt gagccaccgt gcccagccta attttttgat | 8880 |
| tttgatattt gtaaagatga ggtctcactt tgttgcccag gctggtctca agctcatggg | 8940 |
| ctcaagtgat cctcccacct cagcctcctg agtagctggt atcacaggcg caagccactg | 9000 |
| tgctctctcc aataatctta atgctaggat gtacctcgca taatagtttt tgcttacatt | 9060 |
| ttagtttttt tgcacatgtg tgtatatgga atgcaaaatt ggaatcaagt gaatatcttg | 9120 |
| atttatagcc tggatttttt tcacccaata tcaggatcag ctttcctttc caacatacgt | 9180 |
| ccacatcact tttcgtgact gtagaatttc cactggaatg gtttagcaca atttggtcac | 9240 |
| tgagtcttgg tttttagata cttgagattg cttttaaaac ctgaacttaa aaaccacatt | 9300 |
| gtcatgtagg ctgtcttaat gcttcccttt ttttcatcct caattatttt gggggttgat | 9360 |
| ttcctagagt tgaaaattgc tgggtcaaag gcaatgctta ttttttaaaa ctttgggtac | 9420 |
| atattttctt agtttttggg cttgatcctt acctttccaa aattcttttg ctaatccatt | 9480 |
| tattttgtat agqtaataca gtcacaaaat tcaacactaa aaggcataga atggtttaga | 9540 |
| gcaaaaagtc tccctccttc cctggttcat caccacccac tcccctccct agcctttccc | 9600 |
| tagaggcagc cgtgttgtca ttttcttgtt tagacttcct gagagatttt tatgcacatg | 9660 |
| taagcaaata tggaactatc ctttcctcta cttttcataa gaaatgctag atatctgcat | 9720 |
| atttgtctag acttaatact tcttgacgat tgctacatgt cagcttataa acagtttcct | 9780 |
| gctttttctt tttttgtgct gcccagtatt tttcattcaa tgggttggcc gtaatttcac | 9840 |
| cagcccctca ttgatggatg ttgcattggg tattttgggt catcttttac acacagcact | 9900 |
| gctgcgaata actttgtgtg tgcaatattt tgtgtgaatg cttctatgat atttaggatg | 9960 |
| aattcctaga aatcaaatag ctgggttaaa aggtagaaag aaatgttggt aaccctcacc | 9720 |
| tcacctaatt gccattcaaa ataataccaa cagctccagt gtctcgagcc tgtcctatgt | 10080 |
| gcaggtttag ctctgagtgc tttacggaca tcaactcccc tcatcttttt tgagatsggg | 10140 |
| tatcactctg tcgtccaggc tggagtgcag agcataatca tggctcactg cagccttgaa | 10200 |
| ctccccagct caagcastcc tctcacctca gcctcctgag tagctgggac cacaggcacc | 10260 |
| caccactatg cccagctaat tttttgtaaa gacaggagtc tcgttatgtt gcctaggctg | 10320 |
| gactccaact cctgggctca agcagtcctc ccaccttggc ctcccaaaat gttgggatta | 10380 |
| caggtgcgag ccactgtgcc cagccaacat caaccccttg agtcttcgct gccactccat | 10440 |
| gaggtgggca actgtgagga gatcaagaca aagggaggca gtgacatggc ctggcacaca | 10500 |
| gctggcgggg ggcacttcca gattcaaacc cagcctggct ccagggtgct cacccttatc | 10560 |
| ccgcacgctg tcctccttag agatcacact gtttcaccct cccaggagca gggactatgg | 10620 |
| atgcatctca tagcccctag atttcatgta gcttgctggc agcctgaggg ctggggcagg | 10680 |
| actgtggcag actcccacgc tgcaaaccag gtctggggct cagcgaggct cagcctgtag | 10740 |
| ccgaagttct ccccacacag tagagtctcc tatgggacag gaggcagact ggggctgtgg | 10800 |
| cagggatgag gttctctgtg gtcggctatt tcttcctgcc ccaggcctgg gccaaggaga | 10860 |
| cagggaaata caccgaaggc gtggatgaag ccgatccggc caagtggaag gccaacctgc | 10920 |
| gctgtgccct taacaagagc cgggacttcc gcctcatcta cgacgggccc cgggacatgc | 10980 |
| cacctcagcc ctacaagatc tacgaggtct gctccaatgg ccctgctccc acaggtatca | 11040 |
| ggcctagccc tctgtgggcc acctgggagg ctgtgcaatg tcctggcccc cagccatgag | 11100 |
| ctcttgggtg caggcaggcc aagggcccct ctagcaggca gtggtccagg aaacgatgcg | 11160 |
| ggggctcccg ctaggtcatg acacccaggg cttccaggag tggctgggat gggtcactgg | 11220 |
| catatcagga atggcttggc gtgcagtcag ggacctgggt gcttcttcct taccattgcc | 11280 |
| ctcgttttgg cttctggctc cagcctaggt ctcatggccc atggagtcgg ggaggtcttt | 11340 |
| cccaatcctg gtggctgtgc cctccacctc gccctgtgtt gggggcagct ttggggaagg | 11400 |
| cagaagctgc ataggagcta caggcagcct ctcaggggat cttgcttctc ctccgacatt | 11460 |
| gactccttta ctgccctgct tttctctccc tgctgtgcag actcccagcc ccctgaggat | 11520 |
| tactcttttg gtgcaggaga ggaggaggaa gaagaggaag aggtgagtgt gggttgagga | 11580 |
| ggcaggtgga gccctggacg agctctctgc tgtccccatc ggccttaggt ttccgcagcc | 11640 |
| ccactcccat ggagccccgt ggccctctca atagttctcc ttgtttcttc tcctgggatt | 11700 |
| ctgaacgata ggagcacagt ccccacctgc tccttcccag ggcattgtca ttaccctgtg | 11760 |
| tgtgtgaccc acgcagcagt tggggcttgg taggtctgac tccctgcaga aggcaaatga | 11820 |
| ggaaagtgag gcaaagggct tttctgacct gcctgggatg gacgagctgg gaccggaggc | 11880 |
| agggtcttgc ctgagctaaa ctgaggctag gggagttgcc tcatagttct cgcctgttat | 11940 |
| ttccccagcc ccaggtcagt ggaataacct gtcctccttt ctctcccatc tcttccctcc | 12000 |
| cttgctggtg gtgtcccttc agctgcagag gatgttgcca agcctgagcc tcacaggtgg | 12060 |
| ggccgggagg tggtggttgg gggtctagta tacagagaag ctataggtac cataggtacc | 12120 |
| tggaaggggg ctgatgggag gctagggtgg cccagggctg ggaggaggtg tgcctgggag | 12180 |
| gcagttcgtg gaggtggcac tgacagccgt ccacacgcac tctctgtaga tgcagtgcag | 12240 |
| tccggccccc acatgacacc ctattcttta ctcaaagagg atgtcaagtg gccgcccact | 12300 |
| ctgcagccgc ccactctgcg gccgcctact ctgcagccgc ccactctgca gccgcccgtg | 12360 |
| gtgctgggtc cccctgctcc agaccccagc cccctggctc ctccccctgg caaccctgct | 12420 |
| ggcttcaggg agcttctctc tgaggtcctg gagcctgggc ccctgcctgc cagcctgccc | 12480 |
| cctgcaggcg aacagctcct gccagacctg ctgatcagcc cccacatgct gcctcgtaag | 12540 |
| gacccatggc tgggcacggg gaagcagtgc tgggggattg gggtaggatt ggcaaggagg | 12600 |
| gtggagggtg ctggactccc ttgggtggga aaagtgggag ggcggatggg gctgggcctg | 12660 |
| gccactgggc tgcagaatgg ggaggcgtgg ggctcaagga cgggatgggc ctgccttctg | 12720 |
| ccccacagtg accgacctgg agatcaagtt tcagtaccgg gggcggccac cccgggccct | 12780 |
| caccatcagc aacccccatg gctgccggct cttctacagc cagctggagg ccacccagga | 12840 |
| gcaggtggaa ctcttcggcc ccataagcct ggagcaagtg cgcttcccca gccctgagga | 12900 |
| catccccagt gacaagcagc gcttctacac gaaccagctg ctggatgtcc tggaccgcgg | 12960 |
| gctcatcctc cagctacagg gccaggacct ttatgccatc cgcctgtgtc agtgcaaggt | 13020 |
| gttctggagc gggccttgtg cctcagccca tgactcatgc cccaacccca tccagcggga | 13080 |
| ggtcaagacc aagcttttca gcctggagca ttctctcaat ggtgagggcc caaagctgtg | 13140 |
| atcctcctgg ctgcctcttg cccagggcat ggttccagcc tctgactagg gaccttgatt | 13200 |
| ttgatgcaga gctcatcctg ttccaaaagg gccagaccaa caccccacca cccttcgaga | 13260 |
| tcttcttctg ctttggggaa gaatggcctg accgcaaacc ccgagagaag aagctcatta | 13320 |
| ctgtacaggt acatctcccc tatcccaaag tcggccttgg cttgaaaact ggggaatcct | 13380 |
| ggggctaggc ccttgcccca ggctggaggc tcagggctcc ctgagcagtg tgaacttggc | 13440 |
| ggccagagac catcaaggct cagagccgga gaatgcggtc tattactcac ccctgatggc | 13500 |
| tgtcctcatg cacagctgga tctggcagcc ctgccacagg tctccctgtc tcatctcctc | 13560 |
| tttgcctccc aggtggtgcc tgtagcagct cgactgctgc tggagatgtt ctcaggggag | 13620 |
| ctatcttggt cagctgatag tatccggcta cagatctcaa acccagacct caaagaccgc | 13680 |
| atggtggagc aattcaagga gctccatcac atctggcagt cccagcagcg gttgcagcct | 13740 |
| gtggcccagg cccctcctaa agcaggcctt ggtgttggcc aggggccctg gcctatgcac | 13800 |
| ccagctggca tgcaataaca aggctgcaga cggtgactgg ccctggcttc ctgggtggcg | 13860 |
| gtgcggactg atgtggagat gtgacagccc cgatgagcac ctggctggct gcaggatcct | 13920 |
| acctctgggt ttcctggaag tggatttggg ccaagaagga gagggagaaa ggcccgagcc | 13980 |
| cctgccttcc cgggcctttc tctcctgggc tgtctctggt ctggtcagcc tggctctcgg | 14040 |
| gaaattcagc catgagcagg gaaagaactc tcccaaccct ggggcctagc tgtataggag | 14100 |
| gaattgccta agggtggccc actcttgtga ttgccccatt tcctctggca acaaaagcca | 14160 |
| gagtgttgtg ggccaagtcc ccccacaggg cctctgcagg gcatggccct gatttccctg | 14220 |
| gtttgagact cacttcctca tctccctgtc ctctgagata atatgagtca gcacttaggt | 14280 |
| atcatatcag atgctcaagg ctggcagcta cccccttctt gagagtccaa gaacctggag | 14340 |
| cagaaataat ttttatgtat ctttggatta atgaatgtta aaaacagact cagctgtctc | 14400 |
| tttcctttta ctactaccag ttgctcccat gctgctccac caggccctgt ttcggatgcc | 14460 |
| aactggccca ctccccaagc acttgccccc agcttgcgac cattggcact gggagggcct | 14520 |
| ggcttctggg ctgatgggtc agttgggcct tcataaacac tcacctggct ggctttgcct | 14580 |
| tccaggagga agctggctga agcaagggtg tggaatttta aatgtgtgca cagtctggaa | 14640 |
| aactgtcaga atcagttttc ccataaaagg gtgggctagc attgcagctg catttgggac | 14700 |
| cattcaaatc tgtcactctc ttgtgtatat tcctgtgcta ttaaatatat cagggcagtg | 14760 |
| catgtaaatc atcctgatat atttaatata tttatxatat tgtcccccga ggtggggaca | 14820 |
| gtgagtgagt tctcttagtc cccccagagc tggttgttaa agagcctggc acctacccgc | 14880 |
| tctcacttca tctgtgtcat ctctgcacac tccagcccac tttctgcctt cagccattga | 14940 |
| gtggaagctg ccccaggccc ttaccaggtg cagatgccca atcttgatgc ccagccatca | 15000 |
| gaactgtgag ccaaataaac ctttttctgt ataaaccacc cagcctcggg tcttcgttta | 15060 |
| cagcaacgca aaatagatta aacccccata aatgttcaag gataccttgc cccacagcct | 15120 |
| cgtccacaga atatattgtc actgtttgga tttttgccaa cctgacaggt gagacagtat | 15180 |
| ctcagtgcca cttctcatta tcagcaaggc tgagtagctt ttcacatggt taagtggcct | 15240 |
| gtacagattt ttttaaataa ttttagaatg gttttagatt tatggaaaag ctcctaatag | 15300 |
| agttcctatg gacccacact ttctccaatt gttaacatct tacattacta tggcacactt | 15360 |
| gtgacaataa tgaaaccatg tqaacaatta ctatgaactc aacttcttta tttggatttc | 15420 |
| atgagttttt cggatatcct ttttctgttc caggatacta tccaagacat cacattacat | 15480 |
| tcacttgtca catctcctta gactcctctg gtctatgaca gtttcttaaa cttttcttgt | 15540 |
| tgttggtctt gatggttttg aggaatactg gccatttttt aaagcatgtc ccccagttcg | 15600 |
| ggcttatctg atgtatttct catttgtttt tagagatagg gtctttgtgt tgcccaggct | 15660 |
| ggagtgccat ggtgcaacct tgaactcctg ggctcagatg atcctcccac gtagctggga | 15720 |
| ctacgggtgc acaccaccac acctggcttt ttttttttga gacctggtct caatatattg | 15780 |
| cccaggctgg tctcaaactc ctggcctcaa gtgatcttcc caccgtggcc tcccaaacag | 15840 |
| ctgagattac aggtgtgaac cactgtgccc cgcctgtttc tcatggtttt tgtatggaag | 15900 |
| accacagagg tgaagcatca ttcccactac atcatatcaa gggtgcatac tattaacctg | 15960 |
| atttatcact gtttctgact gaggtagtga taggttcctc tatcatagtt actcttcatc | 16020 |
| ctccctgatc tatatgtaca caggtaccgt ggttttagta ttattcacct gcatcctctt | 16080 |
| aaacttcacc aactagagta cagtgattac gtagtgctcc ttttgtctta gttttcaatc | 16140 |
| atttacaaag gtgtgcacct ttttcttcac aggcctgtgc agttctactc cctcttgcaa | 16200 |
| ttgtcaatgt tttgagatta ggagaaggga gatgtcacat atctggtttc cagcttcttc | 16260 |
| caaaaaactg gcaaatatga aaacactgtc atacattcct caaaggcaaa aatcagctgg | 16320 |
| aactctggat ggggaatgtt ctcccagttc aatacggtcc tctcctggcc tgcttccttt | 16380 |
| cactgotctc cgtgtcccta agggcatttg agtttgcaac ccttgatgaa tggtggagca | 16440 |
| gggagactga gtgtagatga attggggaag cttggaaggg ccaggtgccc ctcgcgacca | 16500 |
| cgagtgggct gacttcccca gtactgctag cagttgagta actgccaaaa atcttccctg | 16560 |
| aatttgtatt aattggtttc ctaaaactaa ctaaaactaa gcaatgaaaa cctggctgtc | 16620 |
| acttacagat ctctgtgttg tagtagccag ctgtggatga acagtatcca gaagtgacag | 16680 |
| atgtaacaaa ctccagaggt aaaggcaggc cagtgtgagg tacaattcac tgctcccaag | 16740 |
| ccagactagg gctccccttg gtaatagctt ttggctgctc tggtggcctg tgctggagct | 16800 |
| taagttggaa cccacaggac cttgatgccc acatcctggg gatgaatgca tccctttagc | 16860 |
| cccagcagtt tctctccctt ctttcaagct ccttgtcccc accaagttca tccttataac | 16920 |
| caactaaata tacccttttt aagactctca gctctttgac ataaagaacc taccctggcc | 16980 |
| gggtgcagtg gctcacgcct gtagtcccag cactttggga ggccgaggtg ggcagatcac | 17040 |
| gaggtcagga gttcgagacc agcctggcca acatggtgaa accccatctc cactaasaat | 17100 |
| acaaatatta gctggtcatg gtggtgggtg cctgtaatcc tagctgctca ggaggctgag | 17160 |
| gcaggagaat cgctggaccc catcagacag aggttgcagt gagctgaaat cccaccattg | 17220 |
| cactccagcc tgggcaacag agcaaggctc cctctcaaaa aaaaaaaaac aaaaaaacaa | 17280 |
| aacaaaacaa aacaaaacaa aaggaaaaaa agaaaaaacg tacccttttc agtctctgta | 17340 |
| gagtagaaga tatcaatggc ttttcccatt tttgctgaaa gagcattctt ggaaggcaat | 17400 |
| gttctcattt gctggtaatc tatcccaaag aatcaggcac atcttgttgc aaaatcttga | 17460 |
| acctttaagg tctcttggtt ttggctttca tcttatgatt cacttaaatc agggccacac | 17520 |
| aaggtagaca tttaaataaa ctcatgaatg ttgatgccat tcaaaagcac agtttccttt | 17580 |
| atgtcttagg tgtgcattct cagttactgt tctttataac aattcccttc agatacataa | 17640 |
| ggattacaag ttactgtgtt ttagtacagt cttgctggaa atagccccaa tgctgatgat | 17700 |
| ctgtgttcca cataatcatg gccaacattt catggctaat cctatcacgg aacagacaat | 17760 |
| tctgtgggga gatgacttag gcaagttcag gtgcagttag ctgacctgcc cacatggacg | 17820 |
| ggagatactc atcctatttc tactagacaa tgccatgcag aaacacagca tctccattag | 17880 |
| gtgagctgat cctgaacagt tcctttttaa attaaaatct agatgggcat ccttgctctg | 17940 |
| ccgaaagttt cagagcagcc ctttcaaata atctgtaagg cttaagtaag cagtgaggtt | 18000 |
| <210> 10 | |
| <211> 16 | |
| <212> DNA | |
| <213> Artificial sequence | |
| <220> | |
| <223> Synthetic o1igonuc1eotide | |
| <400> 10 | |
| SEQ ID NO: 10 | |
| cctatacagc taggcc | 16 |
| <210> 11 | |
| <211> 16 | |
| <212> DNA | |
| <213> Artificial sequence | |
| <220> | |
| <223> Synthetic oligonucleotide | |
| SEQ ID NO: 11 | |
| <400> 11 tctgatatga taccta | 16 |
| <210> 12 | |
| <211> 16 | |
| <212> DNA | |
| <213> Artificial sequence | |
| <220> | |
| <223> Synthetic oligonucleotide | |
| <400> 12 | |
| SEQ ID NO: 12 | |
| tatttctgct ccaggt | |
| <210> 13 | |
| <211> 16 | |
| <212> DNA | |
| <213> Artificial, sequence | |
| <220> | |
| <223> Synthetic oligonucleotide | |
| <400> 13 | |
| SEQ ID NO: 13 | |
| ctgatatgat acctaa | 16 |
| <210> 14 | |
| <211> 16 | |
| <212> DNA | |
| <213> Artificial sequence | |
| <220> | |
| <223> Synthetic oligonucleotide | |
| <400> 14 | |
| SEQ ID NO: 14 | |
| acgagttatg ggaagg | 16 |
| <210> 15 | |
| <211> 16 | |
| <212> DNA | |
| <213> Artificial sequence | |
| <220> | |
| <223> Synthetic oligonucleotide | |
| <400> 15 | |
| SEQ ID NO: 15 | |
| atgagtaata ggagtg | 16 |
| <210> 16 | |
| <211> 16 | |
| <212> DNA | |
| <213> Artificial sequence | |
| <220> | |
| <223> Synthetic oligonucleotide | |
| <400> 16 | |
| SEQ ID NO: 16 | |
| tgtctagtgt catgga | 16 |

1. A method of treating a human subject having a disease associated with Interferon Regulatory Factor 5 (IRF5), comprising:
a. administering to the human subject a safe and effective amount of an antisense oligomer to an IRF 5 nucleic acid.

2. The method of claim 1, wherein the antisense oligomer is administered in an amount of about 20 mg/day to about 1500 mg/day.

3. The method of claim 1, wherein the antisense oligomer is administered as a once daily oral administration of 120 mg/day, 360 mg/day, or 720 mg/day.

4. (canceled)

5. (canceled)

6. The method of claim 1, wherein the antisense oligomer is administered once every 2, 3, 4, 5, 6, 7, 8, 9, 10, 11, 12, 12 or 14 days.

7. The method of claim 1, wherein the antisense oligomer is administered twice or three times daily.

8. The method of claim 1, wherein the antisense oligomer is administered for a treatment cycle of about 1 week, 2 weeks or about 3 weeks.

9. The method of claim 8, wherein the treatment cycle is repeated after a break period in a range of about 6 to 10 weeks.

10. The method of claim 8, wherein the treatment cycle is repeated two times, three times, four times, or five times.

11. (canceled)

12. (canceled)

13. The method of claim 1, wherein the oligonucleotide comprises at least 8 contiguous nucleobases that are complementary to an IRF5 nucleic acid.

14. The method of claim 1, wherein the antisense oligomer comprises an oligonucleotide having a nucleobase sequence that is complementary to an IRF5 nucleic acid selected from the group consisting of: SEQ ID NO: 10, SEQ ID NO: 11, SEQ ID NO: 12, SEQ ID NO: 13, SEQ ID NO: 14, SEQ ID NO: 15, and SEQ ID NO: 16.

15. The method of claim 14, wherein the oligonucleotide is a modified oligonucleotide.

16. The method of claim 15, wherein the modified oligonucleotide comprises at least one modification selected from the group consisting of: a modified sugar, a modified internucleoside linkage, a modified nucleobase, and combinations thereof.

17. The method of claim 15, wherein the modified oligonucleotide comprises a modified sugar selected from the group consisting of: a 2′-F and a 2′-O-methoxyethyl modified sugar.

18. The method of claim 15, wherein the modified oligonucleotide comprises a modified sugar selected from the group consisting of: a 4′-CH(CH₃)—O-2′, a 4′-CH₂—O-2′, and a 4′-(CH₂)₂—O-2′ bicyclic sugar.

19. The method of claim 15, wherein the modified oligonucleotide comprises a modified internucleoside linkage selected from the group consisting of: a phosphoramide, a phosphorothioate, and a phosphorodithioate internucleoside linkage.

20. The method of claim 15, wherein the modified oligonucleotide comprises a 5-methylcytosine.

21. The method of claim 15, wherein the modified oligonucleotide comprises a gapmer motif.

22. The method of claim 21, wherein the gapmer motif comprises: a 5′ wing segment consisting of three linked nucleosides, a 3′ wing segment consisting of three linked nucleosides, and a gap segment consisting of ten linked deoxynucleosides that is positioned between the 5′ wing segment and the 3′ wing segment, and wherein each nucleoside of the wing segments comprises a cEt sugar, each internucleoside linkage is a phosphorothioate linkage, and each cytosine is a 5-methylcytosine.

23. The method of claim 21, wherein the modified oligonucleotide has the chemical structure: 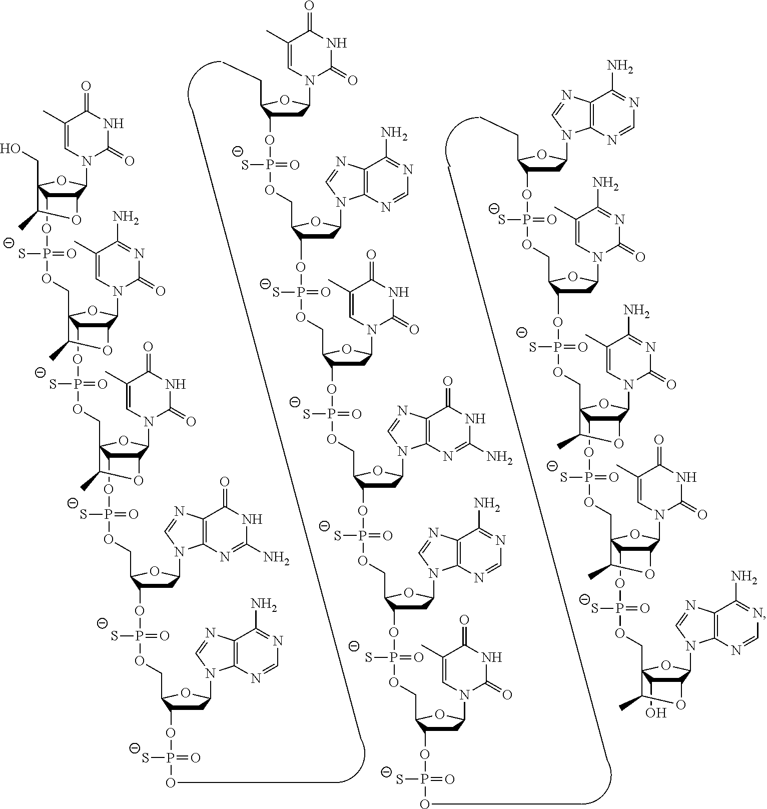
or the sodium salt thereof.

24. The method of claim 1, wherein the disease is ulcerative colitis or Crohn's disease.

25. The method of claim 1, wherein the disease is inflammatory bowel disease, systemic lupus erythematosus, rheumatoid arthritis, primary biliary cirrhosis, systemic sclerosis, Sjogren's syndrome, multiple sclerosis, scleroderma, interstitial lung disease, polycystic kidney disease, chronic kidney disease, nonalcoholic steatohepatitis, liver fibrosis, asthma, or severe asthma.

26. The method of claim 1, wherein the human subject has, or is at risk of having, inflammation, cirrhosis, fibrosis, proteinuria, joint inflammation, autoantibody production, inflammatory cell infiltration, collagen deposits, or inflammatory cytokine production.

27. The method of claim 1, wherein the human subject has, or is at risk of having, inflammation in the gastrointestinal tract, diarrhea, pain, fatigue, abdominal cramping, blood in the stool, intestinal inflammation, disruption of the epithelial barrier of the gastrointestinal tract, dysbiosis, increased bowel frequency, tenesmus or painful spasms of the anal sphincter, constipation, or unintended weight loss.


SEQUENCE LISTING (16 sequences)

SEQ ID NO: 1 - DNA, 2955 bp, Homo sapiens
gcagaaagcggaactgagcccgcgtgttctgaggccagggcagggctggagcgttctgaacacctccccgtcccagcccctgggccaggcaagggccggccttacctctcctgggttggtggcagcagagctgggctctgagggaggcctgcaatgtgagacagtagcagctcagaggcggcactaggcaggtgcaaccccaaaagacccctctgccatgaaccagtccatcccagtggctcccaccccaccccgccgcgtgcggctgaagccctggctggtggcccaggtgaacagctgccagtacccagggcttcaatgggtcaacggggaaaagaaattattctgcatcccctggaggcatgccacaaggcatggtcccagccaggacggagataacaccatcttcaaggcctgggccaaggagacagggaaatacaccgaaggcgtggatgaagccgatccggccaagtggaaggccaacctgcgctgtgcccttaacaagagccgggacttccgcctcatctacgacgggccccgggacatgccacctcagccctacaagatctacgaggtctgctccaatggccctgctcccacagactcccagccccctgaggattactcttttggtgcaggagaggaggaggaagaagaggaagagctgcagaggatgttgccaagcctgagcctcacagaggatgtcaagtggccgcccactctgcagccgcccactctgcggccgcctactctgcagccgcccactctgcagccgcccgtggtgctgggtccccctgctccagaccccagccccctggctcctccccctggcaaccctgctggcttcagggagcttctctctgaggtcctggagcctgggcccctgcctgccagcctgccccctgcaggcgaacagctcctgccagacctgctgatcagcccccacatgctgcctctgaccgacctggagatcaagtttcagtaccgggggcggccaccccgggccctcaccatcagcaacccccatggctgccggctcttctacagccagctggaggccacccaggagcaggtggaactcttcggccccataagcctggagcaagtgcgcttccccagccctgaggacatccccagtgacaagcagcgcttctacacgaaccagctgctggatgtcctggaccgcgggctcatcctccagctacagggccaggacctttatgccatccgcctgtgtcagtgcaaggtgttctggagcgggccttgtgcctcagcccatgactcatgccccaaccccatccagcgggaggtcaagaccaagcttttcagcctggagcattttctcaatgagctcatcctgttccaaaagggccagaccaacaccccaccacccttcgagatcttcttctgctttggggaagaatggcctgaccgcaaaccccgagagaagaagctcattactgtacaggtggtgcctgtagcagctcgactgctgctggagatgttctcaggggagctatcttggtcagctgatagtatccggctacagatctcaaacccagacctcaaagaccgcatggtggagcaattcaaggagctccatcacatctggcagtcccagcagcggttgcagcctgtggcccaggcccctcctggagcaggccttggtgttggccaggggccctggcctatgcacccagctggcatgcaataacaaggctgcagacggtgactggccctggcttcctgggtggcggtgcggactgatgtggagatgtgacagccccgatgagcacctggctggctgcagggtcctacctctgggtttcctggaagtggatttgggccaagaaggagagggagaaaggcccgagcccctgccttcccgggcctttctctcctgggctgtctctggtctggtcagcctggctctcgggaaattcagccatgagcagggaaagaactctcccaaccctggggcctagctgtataggaggaattgcctaagggtggcccactcttgtgattgccccatttcctctggcaacaaaagccagagtgttgtgggccaagtccccccacagggcctctgcagggcatggccctgatttccctggtttgagactcacttcctcatctccctgtcctctgagataatatgagtgagcacttaggtatcatatcagatgctcaaggctggcagctacccccttcttgagagtccaagaacctggagcagaaataatttttatgtatttttggattaatgaatgttaaaaacagactcagctgtttctttccttttactactaccagttgctcccatgctgctccaccaggccctgtttcggatgccaactggcccactccccaagcacttgcccccagcttgcgaccattggcactgggagggcctggcttctgggctgatgggtcagttgggccttcataaacactcacctggctggctttgccttccaggaggaagctggctgaagcaagggtgtggaattttaaatgtgtgcacagtctggaaaactgtcagaatcagttttcccataaaagggtgggctagcattgcagctgcatttgggaccattcaaatctgtcactctcttgtgtatattcctgtgctattaaatatatcagggcagtgcatgtaaatcatcctgatatatttaatatatttattatattgtcccccgaggtggggacagtgagtgagttctcttagtccccccagagctggttgttaaagagcctggcacctacccgctctcacttcatctgtgtcatctctgcacactccagcccactttctgccttcagccattgagtggaagctgccccaggcccttaccaggtgcagatgcccaatcttgatgcccagccatcagaactgtgagccaaataaacctttttctgtataaattacccaaaaaaaaaaaaaaaaaa

SEQ ID NO: 2 - DNA, 12896 bp, Homo sapiens
gagagccaccctcgccaggggtgtaggcaggcgagaggagggcctggagctgtgggtcggccacactgcgccctcatttgtgtgcagccccggaggaccagagtggggaagcaccccaccctctcccagggcccaactgagcactgcagcgggaggtacggggttgtcaaatgacagttttgccattccagattgccaaaagagccagtggccagtctagggcaccgcgccgtctggcatctccctggaggccctgggcctggcccgaggctcagcccggatctgcagttgccaggtcagtgcggggcccggagtggattcgcggggcggggcggggcactgcccgcgcccggagctcagcagcagctgcccaggggcgggggcggcaagacgcggaagtgcccggcaggttggcggaccggcgggaggcgcagcctgggcagagctcagcttggtcccgccgcccggccggtgctccctggcgcagccacgcaggcgcaccgcagacaggtgggtcccggccgccgcgctctcctctctgcgtccgcgcccggcgcgccccgagggtggcgggagcggtgccggctactgcccccaagtctaggcctagactgggccccgcgccccccaggcacctgcgggcggcgggatgaagactggagtagggcggggtccgcgtccagctgcgcctggaaagcgagctcgggggggtgcctacagcagggtgcgcccggccggcctgggacttccaaagcgcctcccacgccccgatcggtttggggtgctggcgcccggggagcccagtgacccaggcggcggagtgggcagcgctgcggggggcgccggctctgctgctctccctccccctcgccatcgcccagaatgggggttcccgggagccccgctggaggctggcttggaccacagaggagcgaggcccgatccttactttcgatgcactcgcccttgctcttaccgggccaccctcaccctttcggaaaagaggttgaggttaaagcgttcatcccccgggatcttcaggccaatggcaggaactgtgcaagagtttgggggaagatggtgtcaggtagaggctgcgtccctgggctcgcggccgggaatggcagactctcgtcccccgagcagcggaaaaggatggggcgcaatagttcctgggctggtttcctcaggtcctgtcccagaacttaagaaggcaacaatgaagaggctaaacgtggaggaaaagtgaggctagcatggccgggatgcgtggggagatggttgtctccggaccccgggaggggcgggagcgggtacctgggagcagaggctggagtgggggactttcccagcctcgcggccacgctgaacacagcagggcgaggaccggggtgctgctcctccagcagcaggaggagaggtccagaggccgactctggaggtgggggtggctccgcgggctggcccagggggtgtgccccagcggagcacgcgggaggggtgggggcggaggggaggggggagcaggggcggaggactgggctgggcctgggctcctcgagggcccagaatggggataagtgaccagaaccagagagggctcggctgtatcgatgtaggaaactgtagcccctcaggaggccctctggcaagacctccccttcctgcccccacccccagtagttatggggcctggggtgctggggctgaggggttccaagagtcaagggaagcactgggaaatcacccctttttatctaaaggccctactttggggtttttcccctgtaccctggtcttcccctaccctgaccctgggaggaagctgaaagaagcttctttctgggcaccttttgccccagagcctcagcctgtctggaccaggtgggcagcagggcccagggtgtgggcagctgacccggaggggtgggatttgggggtcagggcctgtacagggaaccccttgtcctctccctgagctgggtgtgggtttgcaaggagacatgtgacccagaccaaccctgggagcagcagggcgcctgctgtctggccactcttactaggactgctgtggcacttcctcccctagtgggtccctggtgcccatgaattgcagctcctgggtggtggtgggggcactgtctcctgggactccagcatggccctggggtgagctgtgggcttaccccacctcagcaggtcctctagggctgcccactggatgcttcgctgcctcacacaattgtagggacttcctcaggctgttggatttccccaccttccggggctcaggtccattgacttaggtctagggctccatacatttcacccagagactccggagcctggcaggcagacctgttctgacaccgaacttccaaagtcatgggccttgattggggtggtctgaattagacctagcccttttctgggcagaagggagcttctaggaggatggatgctgttcgggttagagctcgtgtggacctagctgcaggcaaaagccttgaggctgagtcccttctgtggcatggtggacagactctcgctcatcacagccgggcttgtcacgggagctcctcctccacacccctccctaagctgcctgtatggacgcggccctctgacactgaggtcggagttatcatttcaaaaccttgctctgtattaaacagccgtgttgggcagggccagactgctggactgacagtagggggcaggcagccggaccctctgagctccccaacggcaccagcgcctgcacggcctcagcccagggggtcattagggaagctctccccgattctgtgcagacagagcttcctctgtccacccttgctcggccagaattgtgtgccgctggtgactggcacccctctattctagggccaaggcctctcaggggtctacagatacaactatgggtgggtgcacacccatgttataaaccacactaaatgcacaaaaactgtctgcaagtttggggtgcggggaacagctctgggtgggaggttggaaatttggtctgggggacccactcggctccctccctcagcccacagtgagtctggtttctgagttgtcccggtctagccactttcgtttcccctggggccgggtggaggctggggcagaaagcggaactgagcccgcgtgttctgaggccagggcagggctggagcgttctgaacacctccccgtcccagcccctgggccaggcaagggccggccttacctctcctgggttggtggcagcagagctgggctctgagggaggcctgcaatgtgagacagtagcagctcagaggcggcactaggcaggtgcaaccccaaaaggtactgggcagggaatttcaggggaaactgaggctccatgctgcaaggccaaagcaggcccagacacagggagttcagctcgaacgtttgctccttacttgctgaccgacacattcacttctgatgggctgtgctgtgtaatgacccagtcctttctgtcttccacccaggggtctcaagtgaagggccaagggcatgggtaaggggaggagagggacagaaggggactcaggactgtggagagtttccggttctgggctggagagggagttgggcaggtgaaggggaggaggcagggtccctctggcctgactccagggaggcagtgaagctgtggcctgggaggcggggctcgtgcccctgggtaggtagcagttagagctgtggcttctgtttcctgtagccttcctaacaggctccggcaggcgttagggccttcctaagtgctacccgaatgcgtgtccagtgggaggagaagggggaggaccaagaacctagatgaatggccctagagaggtaacatctgtttggtgcctgctgtcattggaggtgggttgagttggttgatgggatttctctaaagatattgagaaactgctttcctctgagggcaggtgtccttgggagctccagatgagattcttgttgagggcatctccggagggaactctcaggggatggagggtgagaaaacttgggcagggaagaatcgaactaggggtgtagactgagcaggaggctgtcttcgggctgatcccacagagcgctcgctgtcccaggtagccagcttgagacaagacagccgggcttttggtgtcaggcagtcactggctgtgggctgccccccaagagcaaggggtgggcctaacctgtggtgtgagtagtggaaggcggttctctggccaacgggctgcttgctgctgttagcagttggagaatggatgcctctgcccggtaaagggcacctggggccgcgcccgcagcattcactaactcgtaactctccttcccttcctccaaacacaaaattccatgacactagacaagaaagctgatgcttggaaaagagaattggccttaaatacctagatggactggagagaccatcctttgtggtccatagcttgacctctgagtaccctgtccccatccacctgcagacctgctgaggctcccctctggctttctcctgcagacccctctgccatgaaccagtccatcccagtggctcccaccccaccccgccgcgtgcggctgaagccctggctggtggcccaggtgaacagctgccagtacccagggcttcaatgggtcaacggggaaaagaaattattctgcatcccctggaggcatgccacaaggcatggtcccagccaggacggagataacaccatcttcaaggtaagccccggggaggaggttggctggacctccagggcaccctgtccccagaagaggagcgcacataacgcacacaggcagctcctcgaggctggccacccgcccagctaccatgctgctgctgatgccgggcccggactaaggggatgcagacgtagacacagggtacacctttttcctttttttttttttttaagacagagtctcgctctgtagcccaggctggagtgcagtggcacgatctcagctcactgcaacctctgcctcctgggttcaagcaattctcctgcctcagcctcctgagtagctgggattacaggcatgagccaccacgcctggcctagggcacatcttttctaacctgcaccctagagcatcgtggggactgagggtccccagaaggccttcccataactcgtcctactcaccctttgctcgtctcactcctattactcatgaggacttgttcagtgcacgcatatgctaaaggaagccaacgatcatcatctttctaaaaattttatttttaaattagtatatatttatggggtacacagtggtgatttgataagcacagtgatcagatcaggtacttagcatatccataatctcaaacatttgtcatttctttgtgttgggaacaaactatttttaatataagattcagatacatcatcaatctttcaattgtttaaattttctaattttttttagagacagggcctcacactgttgaccagattccaattttttttttttttttttttttttttttgagacagggtctcactctgtcacccaggctggagtgcagtggcttgatctcagctcactgcagcctcgacctcccaggctcaggtgagtctcccatctcaacctccttgagtagctgggattacaggtgcctgccaccacaactggctaattttttgtacttttagtagagactttgccatgttgcccaggctggtcttgaactcctggactcaagcaatccacccacctcagcctcccagagtgctgggattacaggtgtgagccaccatgcccggcccaatctttttttttaattgatgtactacaactgtacatatatactctttttttttttttttgagatgagttgttgctctgttgcctagtgttgcaccattggtgagcagtggtgcagtcatagcacaacctccaactcagctcaagtgatcctccagtctcagcttcctgagtagccgtgactacaggtgcatgccaccatgcccagctaatttttttttttttttttgagatggagtcttgctttttcaccctagctggagtgcaatggcacaatctcagctcactgcaacctctgccacctgggttcaagcaattctcctgcctcagcctcccgagtagctaggattacaggcacctgccaccatgcccggctaatttttttttttttttttttttttttttttttttttgtatttttggtaaagacagggtttcaccctgttggccaggctggtcttgaactcctgacctcatgatccacctgccttggcctcccaaagtgctgggattacaggcgtgagccaccgtgcccagcctaattttttgattttgatatttgtaaagatgaggtctcactttgttgcccaggctggtctcaagctcatgggctcaagtgatcctcccacctcagcctcctgagtagctggtatcacaggcgcaagccactgtgctctctccaataatcttaatgctaggatgtacctcgcataatagtttttgcttacattttagtttttttgcacatgtgtgtatatggaatgcaaaattggaatcaagtgaatatcttgatttatagcctggatttttttcacccaatatcaggatcagctttcctttccaacatacgtccacatcacttttcgtgactgtagaatttccactggaatggtttagcacaatttggtcactgagtcttggtttttagatacttgagattgcttttaaaacctgaacttaaaaaccacattgtcatgtaggctgtcttaatgcttccctttttttcatcctcaattattttgggggttgatttcctagagttgaaaattgctgggtcaaaggcaatgcttattttttaaaactttgggtacatattttcttagtttttgggcttgatccttacctttccaaaattcttttgctaatccatttattttgtataggtaatacagtcacaaaattcaacattaaaaggcatagaatggtttagagcaaaaagtctccctccttccctggttcatcaccacccactcccctccctagcctttccctagaggcagccgtgttgtcattttcttgtttagccttcctgagagatttttatgcacatgtaagcaaatatggaactatcctttcctctacttttcataagaaatgctagatatctgcatatttgtctagacttaatacttcttgacgattgctacatgtcagcttataaacagtttcctgctttttctttttttgtgctgcccagtatttttcattcaatgggttggccgtaatttcaccagcccctcattgatggatgttgcattgggtattttgggtcatcttttacacacagcactgctgcgaataactttgtgtgtgcaatattttgtgtgaatgcttctatgatatttaggatgaattcctagaaatcaaatagctgggttaaaaggtagaaagaaatgttggtaaccctcacctcacctaattgccattcaaaataataccaacagctccagtgtctcgagcctgtcctatgtgcaggtttagctctgagtgctttacggacatcaactcccctcatcttttttgagatagggtatcactctgtcgtccaggctggagtgcagagcataatcatggctcactgcagccttgaactccccagctcaagcaatcctctcacctcagcctcctgagtagctgggaccacaggcacccaccactatgcccagctaattttttgtaaagacaggagtctcgttatgttgcctaggctggactccaactcctgggctcaagcagtcctcccaccttggcctcccaaaatgttgggattacaggtgcgagccactgtgcccagccaacatcaaccccttgagtcttcgctgccactccatgaggtgggcaactgtgaggagatcaagacaaagggaggcagtgacatggcctggcacacagctggcggggggcacttccagattcaaacccagcctggctccagggtgctcacccttatcccgcacgctgtcctccttagagatcacactgtttcaccctcccaggagcagggactatggatgcatctcatagcccctagatttcatgtagcttgctggcagcctgagggctggggcaggactgtggcagactcccacgctgcaaaccaggtctggggctcagcgaggctcagcctgtagccgaagttctccccacacagtagagtctcctatgggacaggaggcagactggggctgtggcagggatgaggttctctgtggtcggctatttcttcctgccccaggcctgggccaaggagacagggaaatacaccgaaggcgtggatgaagccgatccggccaagtggaaggccaacctgcgctgtgcccttaacaagagccgggacttccgcctcatctacgacgggccccgggacatgccacctcagccctacaagatctacgaggtctgctccaatggccctgctcccacaggtatcaggcctagccctctgtgggccacctgggaggctgtgcaatgtcctggcccccagccatgagctcttgggtgcaggcaggccaagggcccctctagcaggcagtggtccaggaaacgatgcgggggctcccgctaggtcatgacacccagggcttccaggagtggctgggatgggtcactggcatatcaggaatggcttggcgtgcagtcagggacctgggtgcttcttccttaccattgccctcgttttggcttctggctccagcctaggtctcatggcccatggagtcggggaggtctttcccaatcctggtggctgtgccctccacctcgccctgtgttgggggcagctttggggaaggcagaagctgcataggagctacaggcagcctctcaggggatcttgcttctcctccgacattgactcctttactgccctgcttttctctccctgctgtgcagactcccagccccctgaggattactcttttggtgcaggagaggaggaggaagaagaggaagaggtgagtgtgggttgaggaggcaggtggagccctggacgagctctctgctgtccccatcggccttaggtttccgcagccccactcccatggagccccgtggccctctcaatagttctccttgtttcttctcctgggattctgaacgataggagcacagtccccacctgctccttcccagggcattgtcattaccctgtgtgtgtgacccacgcagcagttggggcttggtaggtctgactccctgcagaaggcaaatgaggaaagtgaggcaaagggcttttctgacctgcctgggatggacgagctgggaccggaggcagggtcttgcctgagctaaactgaggctaggggagttgcctcatagttctcgcctgttatttccccagccccaggtcagtggaataacctgtcctcctttctctcccatctcttccctcccttgctggtggtgtcccttcagctgcagaggatgttgccaagcctgagcctcacaggtggggccgggaggtggtggttgggggtctagtatacagagaagctataggtaccataggtacctggaagggggctgatgggaggctagggtggcccagggctgggaggaggtgtgcctgggaggcagttcgtggaggtggcactgacagccgtccacacgcactctctgtagatgcagtgcagtctggcccccacatgacaccctattctttactcaaagaggatgtcaagtggccgcccactctgcagccgcccactctgcggccgcctactctgcagccgcccactctgcagccgcccgtggtgctgggtccccctgctccagaccccagccccctggctcctccccctggcaaccctgctggcttcagggagcttctctctgaggtcctggagcctgggcccctgcctgccagcctgccccctgcaggcgaacagctcctgccagacctgctgatcagcccccacatgctgcctcgtaaggacccatggctgggcacggggaagcagtgctgggggattggggtaggattggcaaggagggtggagggtgctggactcccttgggtgggaaaagtgggagggcggatggggctgggcctggccactgggctgcagaatggggaggcgtggggctcaaggacgggatgggcctgccttctgccccacagtgaccgacctggagatcaagtttcagtaccgggggcggccaccccgggccctcaccatcagcaacccccatggctgccggctcttctacagccagctggaggccacccaggagcaggtggaactcttcggccccataagcctggagcaagtgcgcttccccagccctgaggacatccccagtgacaagcagcgcttctacacgaaccagctgctggatgtcctggaccgcgggctcatcctccagctacagggccaggacctttatgccatccgcctgtgtcagtgcaaggtgttctggagcgggccttgtgcctcagcccatgactcatgccccaaccccatccagcgggaggtcaagaccaagcttttcagcctggagcattttctcaatggtgagggcccaaagctgtgatcctcctggctgcctcttgcccagggcatggttccagcctctgactagggaccttgattttgatgcagagctcatcctgttccaaaagggccagaccaacaccccaccacccttcgagatcttcttctgctttggggaagaatggcctgaccgcaaaccccgagagaagaagctcattactgtacaggtacatctcccctatcccaaagtcggccttggcttgaaaactggggaatcctggggctaggcccttgccccaggctggaggctcagggctccctgagcagtgtgaacttggcggccagagaccatcaaggctcagagccggagaatgcggtctattactcacccctgatggctgtcctcatgcacagctggatctggcagccctgccacaggtctccctgtctcatctcctctttgcctcccaggtggtgcctgtagcagctcgactgctgctggagatgttctcaggggagctatcttggtcagctgatagtatccggctacagatctcaaacccagacctcaaagaccgcatggtggagcaattcaaggagctccatcacatctggcagtcccagcagcggttgcagcctgtggcccaggcccctcctggagcaggccttggtgttggccaggggccctggcctatgcacccagctggcatgcaataacaaggctgcagacggtgactggccctggcttcctgggtggcggtgcggactgatgtggagatgtgacagccccgatgagcacctggctggctgcagggtcctacctctgggtttcctggaagtggatttgggccaagaaggagagggagaaaggcccgagcccctgccttcccgggcctttctctcctgggctgtctctggtctggtcagcctggctctcgggaaattcagccatgagcagggaaagaactctcccaaccctggggcctagctgtataggaggaattgcctaagggtggcccactcttgtgattgccccatttcctctggcaacaaaagccagagtgttgtgggccaagtccccccacagggcctctgcagggcatggccctgatttccctggtttgagactcacttcctcatctccctgtcctctgagataatatgagtgagcacttaggtatcatatcagatgctcaaggctggcagctacccccttcttgagagtccaagaacctggagcagaaataatttttatgtatttttggattaatgaatgttaaaaacagactcagctgtttctttccttttactactaccagttgctcccatgctgctccaccaggccctgtttcggatgccaactggcccactccccaagcacttgcccccagcttgcgaccattggcactgggagggcctggcttctgggctgatgggtcagttgggccttcataaacactcacctggctggctttgccttccaggaggaagctggctgaagcaagggtgtggaattttaaatgtgtgcacagtctggaaaactgtcagaatcagttttcccataaaagggtgggctagcattgcagctgcatttgggaccattcaaatctgtcactctcttgtgtatattcctgtgctattaaatatatcagggcagtgcatgtaaatcatcctgatatatttaatatatttattatattgtcccccgaggtggggacagtgagtgagttctcttagtccccccagagctggttgttaaagagcctggcacctacccgctctcacttcatctgtgtcatctctgcacactccagcccactttctgccttcagccattgagtggaagctgccccaggcccttaccaggtgcagatgcccaatcttgatgcccagccatcagaactgtgagccaaataaacctttttctgtataaattacccagcctcgggtcttcgtttacagcaacgcaaaatagattaaacccccataaatgttcaaggataccttgccccacagcctcgtccacagaatatattgtcactgtttggatttttgccaacctgacaggtgagatagtatctcagtgccacttctcattatcagcaaggctgagtagcttttcacatggttaagtggcctgtacagatttttttaaataattttagaatggttttagatttatggaaaagttcctaatagagttcctatggacccacactttctccaattgttaacatcttacattactatggcacacttgtgacaataatgaaaccatgtggacaattactatgaactcaacttctttatttggatttcatgagtttttcggat

SEQ ID NO: 3 - DNA, 2910 bp, Homo sapiens
agtgcccggcaggttggcggaccggcgggaggcgcagcctgggcagagctcagcttggtcccgccgcccggccggtgctccctggcgcagccacgcaggcgcaccgcagacagacccctctgccatgaaccagtccatcccagtggctcccaccccaccccgccgcgtgcggctgaagccctggctggtggcccaggtgaacagctgccagtacccagggcttcaatgggtcaacggggaaaagaaattattctgcatcccctggaggcatgccacaaggcatggtcccagccaggacggagataacaccatcttcaaggcctgggccaaggagacagggaaatacaccgaaggcgtggatgaagccgatccggccaagtggaaggccaacctgcgctgtgcccttaacaagagccgggacttccgcctcatctacgacgggccccgggacatgccacctcagccctacaagatctacgaggtctgctccaatggccctgctcccacagactcccagccccctgaggattactcttttggtgcaggagaggaggaggaagaagaggaagagctgcagaggatgttgccaagcctgagcctcacagatgcagtgcagtctggcccccacatgacaccctattctttactcaaagaggatgtcaagtggccgcccactctgcagccgcccactctgcggccgcctactctgcagccgcccactctgcagccgcccgtggtgctgggtccccctgctccagaccccagccccctggctcctccccctggcaaccctgctggcttcagggagcttctctctgaggtcctggagcctgggcccctgcctgccagcctgccccctgcaggcgaacagctcctgccagacctgctgatcagcccccacatgctgcctctgaccgacctggagatcaagtttcagtaccgggggcggccaccccgggccctcaccatcagcaacccccatggctgccggctcttctacagccagctggaggccacccaggagcaggtggaactcttcggccccataagcctggagcaagtgcgcttccccagccctgaggacatccccagtgacaagcagcgcttctacacgaaccagctgctggatgtcctggaccgcgggctcatcctccagctacagggccaggacctttatgccatccgcctgtgtcagtgcaaggtgttctggagcgggccttgtgcctcagcccatgactcatgccccaaccccatccagcgggaggtcaagaccaagcttttcagcctggagcattttctcaatgagctcatcctgttccaaaagggccagaccaacaccccaccacccttcgagatcttcttctgctttggggaagaatggcctgaccgcaaaccccgagagaagaagctcattactgtacaggtggtgcctgtagcagctcgactgctgctggagatgttctcaggggagctatcttggtcagctgatagtatccggctacagatctcaaacccagacctcaaagaccgcatggtggagcaattcaaggagctccatcacatctggcagtcccagcagcggttgcagcctgtggcccaggcccctcctggagcaggccttggtgttggccaggggccctggcctatgcacccagctggcatgcaataacaaggctgcagacggtgactggccctggcttcctgggtggcggtgcggactgatgtggagatgtgacagccccgatgagcacctggctggctgcagggtcctacctctgggtttcctggaagtggatttgggccaagaaggagagggagaaaggcccgagcccctgccttcccgggcctttctctcctgggctgtctctggtctggtcagcctggctctcgggaaattcagccatgagcagggaaagaactctcccaaccctggggcctagctgtataggaggaattgcctaagggtggcccactcttgtgattgccccatttcctctggcaacaaaagccagagtgttgtgggccaagtccccccacagggcctctgcagggcatggccctgatttccctggtttgagactcacttcctcatctccctgtcctctgagataatatgagtgagcacttaggtatcatatcagatgctcaaggctggcagctacccccttcttgagagtccaagaacctggagcagaaataatttttatgtatttttggattaatgaatgttaaaaacagactcagctgtttctttccttttactactaccagttgctcccatgctgctccaccaggccctgtttcggatgccaactggcccactccccaagcacttgcccccagcttgcgaccattggcactgggagggcctggcttctgggctgatgggtcagttgggccttcataaacactcacctggctggctttgccttccaggaggaagctggctgaagcaagggtgtggaattttaaatgtgtgcacagtctggaaaactgtcagaatcagttttcccataaaagggtgggctagcattgcagctgcatttgggaccattcaaatctgtcactctcttgtgtatattcctgtgctattaaatatatcagggcagtgcatgtaaatcatcctgatatatttaatatatttattatattgtcccccgaggtggggacagtgagtgagttctcttagtccccccagagctggttgttaaagagcctggcacctacccgctctcacttcatctgtgtcatctctgcacactccagcccactttctgccttcagccattgagtggaagctgccccaggcccttaccaggtgcagatgcccaatcttgatgcccagccatcagaactgtgagccaaataaacctttttctgtataaattacccaaaaaaaaaaaaaaaaaa

SEQ ID NO: 4 - DNA, 2187 bp, Homo sapiens
gcggcgggaggcgcagcctgggcagagctcagcttggtcccgccgcccggccggtgctccctggcgcagccacgcaggcgcaccgcagacagacccctctgccatgaaccagtccatcccagtggctcccaccccaccccgccgcgtgcggctgaagccctggctggtggcccaggtgaacagctgccagtacccagggcttcaatgggtcaacggggaaaagaaattattctgcatcccctggaggcatgccacaaggcatggtcccagccaggacggagataacaccatcttcaaggcctgggccaaggagacagggaaatacaccgaaggcgtggatgaagccgatccggccaagtggaaggccaacctgcgctgtgcccttaacaagagccgggacttccgcctcatctacgacgggccccgggacatgccacctcagccctacaagatctacgaggtctgctccaatggccctgctcccacagactcccagccccctgaggattactcttttggtgcaggagaggaggaggaagaagaggaagagctgcagaggatgttgccaagcctgagcctcacagatgcagtgcagtctggcccccacatgacaccctattctttactcaaagaggatgtcaagtggccgcccactctgcagccgcccactctgcagccgcccgtggtgctgggtccccctgctccagaccccagccccctggctcctccccctggcaaccctgctggcttcagggagcttctctctgaggtcctggagcctgggcccctgcctgccagcctgccccctgcaggcgaacagctcctgccagacctgctgatcagcccccacatgctgcctctgaccgacctggagatcaagtttcagtaccgggggcggccaccccgggccctcaccatcagcaacccccatggctgccggctcttctacagccagctggaggccacccaggagcaggtggaactcttcggccccataagcctggagcaagtgcgcttccccagccctgaggacatccccagtgacaagcagcgcttctacacgaaccagctgctggatgtcctggaccgcgggctcatcctccagctacagggccaggacctttatgccatccgcctgtgtcagtgcaaggtgttctggagcgggccttgtgcctcagcccatgactcatgccccaaccccatccagcgggaggtcaagaccaagcttttcagcctggagcattttctcaatgagctcatcctgttccaaaagggccagaccaacaccccaccacccttcgagatcttcttctgctttggggaagaatggcctgaccgcaaaccccgagagaagaagctcattactgtacaggtggtgcctgtagcagctcgactgctgctggagatgttctcaggggagctatcttggtcagctgatagtatccggctacagatctcaaacccagacctcaaagaccgcatggtggagcaattcaaggagctccatcacatctggcagtcccagcagcggttgcagcctgtggcccaggcccctcctggagcaggccttggtgttggccaggggccctggcctatgcacccagctggcatgcaataacaaggctgcagacggtgactggccctggcttcctgggtggcggtgcggactgatgtggagatgtgacagccccgatgagcacctggctggctgcagggtcctacctctgggtttcctggaagtggatttgggccaagaaggagagggagaaaggcccgagcccctgccttcccgggcctttctctcctgggctgtctctggtctggtcagcctggctctcgggaaattcagccatgagcagggaaagaactctcccaaccctggggcctagctgtataggaggaattgcctaagggtggcccactcttgtgattgccccatttcctctggcaacaaaagccagagtgttgtgggccaagtccccccacagggcctctgcagggcatggccctgatttccctggtttgagactcacttcctcatctccctgtcctctgagataatatgagtgagcacttaggtatcatatcagatgctcaaggctggcagctacccccttcttgagagtccaagaacctggagcagaaataatttttatgtatttttggattaataaatgttaaaaacag

SEQ ID NO: 5 - DNA, 556 bp, Homo sapiens
gcagaaagcggaactgagcccgcgtgttctgaggccagggcagggctggagcgttctgaacacctccccgtcccagcccctgggccaggcaagggccggccttacctctcctgggttggtggcagcagagctgggctctgagggaggcctgcaatgtgagacagtagcagctcagaggcggcactaggcaggtgcaaccccaaaagacccctctgccatgaaccagtccatcccagtggctcccaccccaccccgccgcgtgcggctgaagccctggctggtggcccaggtgaacagctgccagtacccagggcttcaatgggtcaacggggaaaagaaattattctgcatcccctggaggcatgccacaaggcatggtcccagccaggacggagataacaccatcttcaaggcctgggccaaggagacagggaaatacaccgaaggcgtggatgaagccgatccggccaagtggaaggccaacctgcgctgtgcccttaacaagagccgggacttccgcctcatctacgacgggccccgggacatgccacctcag

SEQ ID NO: 6 - DNA, 2862 bp, Homo sapiens
agtgcccggcaggttggcggaccggcgggaggcgcagcctgggcagagctcagcttggtcccgccgcccggccggtgctccctggcgcagccacgcaggcgcaccgcagacagacccctctgccatgaaccagtccatcccagtggctcccaccccaccccgccgcgtgcggctgaagccctggctggtggcccaggtgaacagctgccagtacccagggcttcaatgggtcaacggggaaaagaaattattctgcatcccctggaggcatgccacaaggcatggtcccagccaggacggagataacaccatcttcaaggcctgggccaaggagacagggaaatacaccgaaggcgtggatgaagccgatccggccaagtggaaggccaacctgcgctgtgcccttaacaagagccgggacttccgcctcatctacgacgggccccgggacatgccacctcagccctacaagatctacgaggtctgctccaatggccctgctcccacagactcccagccccctgaggattactcttttggtgcaggagaggaggaggaagaagaggaagagctgcagaggatgttgccaagcctgagcctcacagaggatgtcaagtggccgcccactctgcagccgcccactctgcggccgcctactctgcagccgcccactctgcagccgcccgtggtgctgggtccccctgctccagaccccagccccctggctcctccccctggcaaccctgctggcttcagggagcttctctctgaggtcctggagcctgggcccctgcctgccagcctgccccctgcaggcgaacagctcctgccagacctgctgatcagcccccacatgctgcctctgaccgacctggagatcaagtttcagtaccgggggcggccaccccgggccctcaccatcagcaacccccatggctgccggctcttctacagccagctggaggccacccaggagcaggtggaactcttcggccccataagcctggagcaagtgcgcttccccagccctgaggacatccccagtgacaagcagcgcttctacacgaaccagctgctggatgtcctggaccgcgggctcatcctccagctacagggccaggacctttatgccatccgcctgtgtcagtgcaaggtgttctggagcgggccttgtgcctcagcccatgactcatgccccaaccccatccagcgggaggtcaagaccaagcttttcagcctggagcattttctcaatgagctcatcctgttccaaaagggccagaccaacaccccaccacccttcgagatcttcttctgctttggggaagaatggcctgaccgcaaaccccgagagaagaagctcattactgtacaggtggtgcctgtagcagctcgactgctgctggagatgttctcaggggagctatcttggtcagctgatagtatccggctacagatctcaaacccagacctcaaagaccgcatggtggagcaattcaaggagctccatcacatctggcagtcccagcagcggttgcagcctgtggcccaggcccctcctggagcaggccttggtgttggccaggggccctggcctatgcacccagctggcatgcaataacaaggctgcagacggtgactggccctggcttcctgggtggcggtgcggactgatgtggagatgtgacagccccgatgagcacctggctggctgcagggtcctacctctgggtttcctggaagtggatttgggccaagaaggagagggagaaaggcccgagcccctgccttcccgggcctttctctcctgggctgtctctggtctggtcagcctggctctcgggaaattcagccatgagcagggaaagaactctcccaaccctggggcctagctgtataggaggaattgcctaagggtggcccactcttgtgattgccccatttcctctggcaacaaaagccagagtgttgtgggccaagtccccccacagggcctctgcagggcatggccctgatttccctggtttgagactcacttcctcatctccctgtcctctgagataatatgagtgagcacttaggtatcatatcagatgctcaaggctggcagctacccccttcttgagagtccaagaacctggagcagaaataatttttatgtatttttggattaatgaatgttaaaaacagactcagctgtttctttccttttactactaccagttgctcccatgctgctccaccaggccctgtttcggatgccaactggcccactccccaagcacttgcccccagcttgcgaccattggcactgggagggcctggcttctgggctgatgggtcagttgggccttcataaacactcacctggctggctttgccttccaggaggaagctggctgaagcaagggtgtggaattttaaatgtgtgcacagtctggaaaactgtcagaatcagttttcccataaaagggtgggctagcattgcagctgcatttgggaccattcaaatctgtcactctcttgtgtatattcctgtgctattaaatatatcagggcagtgcatgtaaatcatcctgatatatttaatatatttattatattgtcccccgaggtggggacagtgagtgagttctcttagtccccccagagctggttgttaaagagcctggcacctacccgctctcacttcatctgtgtcatctctgcacactccagcccactttctgccttcagccattgagtggaagctgccccaggcccttaccaggtgcagatgcccaatcttgatgcccagccatcagaactgtgagccaaataaacctttttctgtataaattacccaaaaaaaaaaaaaaaaaa

SEQ ID NO: 7 - DNA, 2604 bp, Homo sapiens
agtgcccggcaggttggcggaccggcgggaggcgcagcctgggcagagctcagcttggtcccgccgcccggccggtgctccctggcgcagccacgcaggcgcaccgcagacagacccctctgccatgaaccagtccatcccagtggctcccaccccaccccgccgcgtgcggctgaagccctggctggtggcccaggtgaacagctgccagtacccagggcttcaatgggtcaacggggaaaagaaattattctgcatcccctggaggcatgccacaaggcatggtcccagccaggacggagataacaccatcttcaaggcctgggccaaggagacagggaaatacaccgaaggcgtggatgaagccgatccggccaagtggaaggccaacctgcgctgtgcccttaacaagagccgggacttccgcctcatctacgacgggccccgggacatgccacctcagccctacaagatctacgaggtctgctccaatggccctgctcccacagactcccagccccctgaggattactcttttggtgcaggagaggaggaggaagaagaggaagagctgcagaggatgttgccaagcctgagcctcacagtgaccgacctggagatcaagtttcagtaccgggggcggccaccccgggccctcaccatcagcaacccccatggctgccggctcttctacagccagctggaggccacccaggagcaggtggaactcttcggccccataagcctggagcaagtgcgcttccccagccctgaggacatccccagtgacaagcagcgcttctacacgaaccagctgctggatgtcctggaccgcgggctcatcctccagctacagggccaggacctttatgccatccgcctgtgtcagtgcaaggtgttctggagcgggccttgtgcctcagcccatgactcatgccccaaccccatccagcgggaggtcaagaccaagcttttcagcctggagcattttctcaatgagctcatcctgttccaaaagggccagaccaacaccccaccacccttcgagatcttcttctgctttggggaagaatggcctgaccgcaaaccccgagagaagaagctcattactgtacaggtggtgcctgtagcagctcgactgctgctggagatgttctcaggggagctatcttggtcagctgatagtatccggctacagatctcaaacccagacctcaaagaccgcatggtggagcaattcaaggagctccatcacatctggcagtcccagcagcggttgcagcctgtggcccaggcccctcctggagcaggccttggtgttggccaggggccctggcctatgcacccagctggcatgcaataacaaggctgcagacggtgactggccctggcttcctgggtggcggtgcggactgatgtggagatgtgacagccccgatgagcacctggctggctgcagggtcctacctctgggtttcctggaagtggatttgggccaagaaggagagggagaaaggcccgagcccctgccttcccgggcctttctctcctgggctgtctctggtctggtcagcctggctctcgggaaattcagccatgagcagggaaagaactctcccaaccctggggcctagctgtataggaggaattgcctaagggtggcccactcttgtgattgccccatttcctctggcaacaaaagccagagtgttgtgggccaagtccccccacagggcctctgcagggcatggccctgatttccctggtttgagactcacttcctcatctccctgtcctctgagataatatgagtgagcacttaggtatcatatcagatgctcaaggctggcagctacccccttcttgagagtccaagaacctggagcagaaataatttttatgtatttttggattaatgaatgttaaaaacagactcagctgtttctttccttttactactaccagttgctcccatgctgctccaccaggccctgtttcggatgccaactggcccactccccaagcacttgcccccagcttgcgaccattggcactgggagggcctggcttctgggctgatgggtcagttgggccttcataaacactcacctggctggctttgccttccaggaggaagctggctgaagcaagggtgtggaattttaaatgtgtgcacagtctggaaaactgtcagaatcagttttcccataaaagggtgggctagcattgcagctgcatttgggaccattcaaatctgtcactctcttgtgtatattcctgtgctattaaatatatcagggcagtgcatgtaaatcatcctgatatatttaatatatttattatattgtcccccgaggtggggacagtgagtgagttctcttagtccccccagagctggttgttaaagagcctggcacctacccgctctcacttcatctgtgtcatctctgcacactccagcccactttctgccttcagccattgagtggaagctgccccaggcccttaccaggtgcagatgcccaatcttgatgcccagccatcagaactgtgagccaaataaacctttttctgtataaattacccaaaaaaaaaaaaaaaaaa

SEQ ID NO: 8 - DNA, 2778 bp, Homo sapiens
gtccagctgcgcctggaaagcgagctcggacccctctgccatgaaccagtccatcccagtggctcccaccccaccccgccgcgtgcggctgaagccctggctggtggcccaggtgaacagctgccagtacccagggcttcaatgggtcaacggggaaaagaaattattctgcatcccctggaggcatgccacaaggcatggtcccagccaggacggagataacaccatcttcaaggcctgggccaaggagacagggaaatacaccgaaggcgtggatgaagccgatccggccaagtggaaggccaacctgcgctgtgcccttaacaagagccgggacttccgcctcatctacgacgggccccgggacatgccacctcagccctacaagatctacgaggtctgctccaatggccctgctcccacagactcccagccccctgaggattactcttttggtgcaggagaggaggaggaagaagaggaagagctgcagaggatgttgccaagcctgagcctcacagaggatgtcaagtggccgcccactctgcagccgcccactctgcggccgcctactctgcagccgcccactctgcagccgcccgtggtgctgggtccccctgctccagaccccagccccctggctcctccccctggcaaccctgctggcttcagggagcttctctctgaggtcctggagcctgggcccctgcctgccagcctgccccctgcaggcgaacagctcctgccagacctgctgatcagcccccacatgctgcctctgaccgacctggagatcaagtttcagtaccgggggcggccaccccgggccctcaccatcagcaacccccatggctgccggctcttctacagccagctggaggccacccaggagcaggtggaactcttcggccccataagcctggagcaagtgcgcttccccagccctgaggacatccccagtgacaagcagcgcttctacacgaaccagctgctggatgtcctggaccgcgggctcatcctccagctacagggccaggacctttatgccatccgcctgtgtcagtgcaaggtgttctggagcgggccttgtgcctcagcccatgactcatgccccaaccccatccagcgggaggtcaagaccaagcttttcagcctggagcattttctcaatgagctcatcctgttccaaaagggccagaccaacaccccaccacccttcgagatcttcttctgctttggggaagaatggcctgaccgcaaaccccgagagaagaagctcattactgtacaggtggtgcctgtagcagctcgactgctgctggagatgttctcaggggagctatcttggtcagctgatagtatccggctacagatctcaaacccagacctcaaagaccgcatggtggagcaattcaaggagctccatcacatctggcagtcccagcagcggttgcagcctgtggcccaggcccctcctggagcaggccttggtgttggccaggggccctggcctatgcacccagctggcatgcaataacaaggctgcagacggtgactggccctggcttcctgggtggcggtgcggactgatgtggagatgtgacagccccgatgagcacctggctggctgcagggtcctacctctgggtttcctggaagtggatttgggccaagaaggagagggagaaaggcccgagcccctgccttcccgggcctttctctcctgggctgtctctggtctggtcagcctggctctcgggaaattcagccatgagcagggaaagaactctcccaaccctggggcctagctgtataggaggaattgcctaagggtggcccactcttgtgattgccccatttcctctggcaacaaaagccagagtgttgtgggccaagtccccccacagggcctctgcagggcatggccctgatttccctggtttgagactcacttcctcatctccctgtcctctgagataatatgagtgagcacttaggtatcatatcagatgctcaaggctggcagctacccccttcttgagagtccaagaacctggagcagaaataatttttatgtatttttggattaatgaatgttaaaaacagactcagctgtttctttccttttactactaccagttgctcccatgctgctccaccaggccctgtttcggatgccaactggcccactccccaagcacttgcccccagcttgcgaccattggcactgggagggcctggcttctgggctgatgggtcagttgggccttcataaacactcacctggctggctttgccttccaggaggaagctggctgaagcaagggtgtggaattttaaatgtgtgcacagtctggaaaactgtcagaatcagttttcccataaaagggtgggctagcattgcagctgcatttgggaccattcaaatctgtcactctcttgtgtatattcctgtgctattaaatatatcagggcagtgcatgtaaatcatcctgatatatttaatatatttattatattgtcccccgaggtggggacagtgagtgagttctcttagtccccccagagctggttgttaaagagcctggcacctacccgctctcacttcatctgtgtcatctctgcacactccagcccactttctgccttcagccattgagtggaagctgccccaggcccttaccaggtgcagatgcccaatcttgatgcccagccatcagaactgtgagccaaataaacctttttctgtataaattacccaaaaaaaaaaaaaaaaaa

SEQ ID NO: 9 - DNA, 18000 bp, Homo sapiens
ccctcctcccccctgctccccaccccagccaccccactctacactcgctcacacagcgtagcttctactcgagtcaggggagggtgcctcctaccacaccctcagtctgtgatattccagccaggggaaatgggggaaggggccatctcactgtggggagtggggggcccggacagacctccctgtccccaccgcctcctggcccatcaaggggaaatgggagtgctcacacaagggccgggtgtggcttcacttcccctctacttgcccttccacccccaggatggcactgggaggccaagggatcccagatcctggaaacactggagtgctgctctctgtccttcctttctcctgggccatgtctctggccccagatgattcttggctcaggttctttcagttgagggaagtcaggatctgagccaagctgagcccctgcatagcctctatcccgggagccccatatcaatgctgccggcttggtaacatcatcccccttttagagacgaggaaatgagatttgaggtcctacttaaggccacttggctggaatacagcaaagccaggtgcaagccccttctctgcacgcagcccgtgctgcttcttgaggttcagaggctctaaggaaggggcctcacctgagcaagatggacagtgggaagggggcagagggcagaattctagggtctcacggtccctcagaatgggtgaggagtatttgataacaaaagaaggctcttcttagctattgctccagatacgaccagcatagaatatttttctattaagcataactgacctccagtgaaaaaggttcccattttgtggggagccacttttgaaatagcaatttcattttattattattattttgagataagatctcacttctacgcccaggctggagtgcggtggtgtgcctcctgggttcaagcaattctcccacctcagcctcccaagtaggtggaactataggtgtgtgtcaccacgccaggctaatttttgtattttttgtagagacaaggttttgccatattgcccaggctggtctcgaactcttggactcaagtgatctgcctgccttagcctctcaaagtgttgggattacaaatgtgagccaccgtgcctggcctagcaatttcatttttaaagaaagaaatataaaacgatctacccattcacattttccccatccatactttaaattttaaaaatatgaaataatgaaatactcagcagatacaaataataaaatacttatatataatgatatttcgacttcatattagggcttcaaaggtcaggaaagagggagagatacagaagagaaaaaaaaaaaacgttaacacatgagtgtctttttttaacttgttaaaggtaaagttcaacccaggagaagggaggggagggaaggtcaaagttctgattcatcaacaaatactactttatttgttaaaaatacaaggctctgcttggcgcagtggctcccgcctgtaatcccagcactttgggaggccaaggcgggtggatcgcctgaggtcaggagctcgagaccagcctggccaacatggtgaaaccccgtctctacttgggaggctgaggcaggagaatcgcttgaactggggaggcagaggttgcagtgagctgagatcgcactgctgcactccagcccgggtgacagagcaagactccatatcaaaaaataaaataaaataaaatgaacaatgaaaagacaggagggaggttcctgagtaagctgattcgagtggaaatcagacatcaaaattgaaacccgctgaattttccaaaaagccagatgctcatagaactgaagcttgagacacttacatcagtacacctgctgcctgttgaccaattcctcttccttgtccctcctgttttccttccctgctatataagcccctaactttagtcaaggggagggatggattggaggtttgtctcccctctcaacagctcactggctgatgtcaccggaataaagtcttccctggcaatactcgctgtctcagtgataggctttctgtgcggtgagcagccagacctggaccaaacccctggcaatccataacaaaatcataagcaaaatcaaccaggaagctatttgcaccctggaaccccacctgggaggggaagtcaaggcagactttccagagaaggcgatgctgaaaatttcagaaacagaaattttgaagagcaagagttaccaagcgaagaacattccatgagaaggaacaggaggtgtgtgaaggtggaggttctggggtgaggttctttatggaatcgaaaacggttcagaaccacaggtcgacggtcaaggtagtagtgagaggaggtgggacaggcgaccacgccgctgcccctgggatgactggaaggcgacttaggggagctggggcgagacaggtgcagggtttgaggatgagaaaggcacagagtgactagaggattcccgcctgcaagcacatctggaaggggtgtctggatcctgggggcagcgactgtgttctagggcgagagccaccctcgccaggggtgtaggcaggcgagaggagggcctggagctgtgggtcggccacactgcgccctcatttgtgtgcagccccggaggaccagagtggggaagcaccccaccctctcccagggcccaactgagcactgcagcgggaggtacggggttgtcaaatgacagttttgccattccagattgccaaaagagccagtggccagtctagggcaccgcgccgtctggcatctccctggaggccctgggcctggcccgaggctcagcccggatctgcagttgccaggtcagtgcggggcccggagtggattcgcggggcggggcggggcactgcccgcgcccggagctcagcagcagctgcccaggggcgggggcggcaagacgcggaagtgcccggcaggttggcggaccggcgggaggcgcagcctgggcagagctcagcttggtcccgccgcccggccggtgctccctggcgcagccacgcaggcgcaccgcagacaggtgggtcccggccgccgcgctctcctctctgcgtccgcgcccggcgcgccccgagggtggcgggagcggtgccggctactgcccccaagtctaggcctagactgggccccgcgccccccaggcacctgcgggcggcgggatgaagactggagtagggcggggtccgcgtccagctgcgcctggaaagcgagctcgggtgggtgcctacagcagggtgcgcccggccggcctgggacttccaaagcgcctcccacgccccgatcggtttggggtgctggcgcccggggagcccagtgacccaggcggcggagtgggcagcgctgcggggggcgccggctctgctgctctccctccccctcgccatcgcccagaatgggggttcccgggagccccgctggaggctggcttggaccacagaggagcgaggcccgatccttactttcgatgcactcgcccttgctcttaccgggccaccctcaccctttcggaaaagaggttgaggttaaagcgttcatcccccgggatcttcaggccaatggcaggaactgtgcaagagtttgggggaagatggtgtcaggtagaggctgcgtccctgggctcgcggccgggaatggcagactctcgtcccccgagcagcggaaaaggatggggcgcaatagttcctgggctggtttcctcaggtcctgtcccagaacttaagaaggcaacaatgaagaggctaaacgtggaggaaaagtgaggctagcatggccgggatgcgtggggagatggttgtctccggaccccgggaggggcgggagcgggtacctgggagcagaggctggagtgggggactttcccagcctcgcggccacgctgaacacagcagggcgaggaccggggtgctgctcctccagcagcaggaggagaggtccagaggccgactctggaggtgggggtggctccgcgggctggcccagggggtgtgccccagcggagcacgcgggaggggtgggggcggaggggaggggggagcaggggcggaggactgggctgggcctgggctcctcgagggcccagaatggggataagtgaccagaaccagagagggctcggctgtatcgatgtaggaaactgtagcccctcaggaggccctctggcaagacctccccttcctgcccccacccccagtagttatggggcctggggtgctggggctgaggggttccaagagtcaagggaagcactgggaaatcacccctttttatctaaaggccctactttggggtttttcccctgtaccctggtcttcccctaccctgaccctgggaggaagctgaaagaagcttctttctgggcaccttttgccccagagcctcagcctgtctggaccaggtgggcagcagggcccagggtgtgggcagctgacccggaggggtgggatttgggggtcagggcctgtacagggaaccccttgtcctctccctgagctgggtgtgggtttgcaaggagacatgtgacccagaccaaccctgggagcagcagggcgcctgctgtctggccactcttactaggactgctgtggcacttcctcccctagtgggtccctggtgcccatgaattgcagctcctgggtggtggtgggggcactgtctcctgggactccagcatggccctggggtgagctgtgggcttaccccacctcagcaggtcctctagggctgcccactggatgcttcgctgcctcacacaattgtagggacttcctcaggctgttggatttccccaccttccggggctcaggtccattgacttaggtctagggctccatacatttcacccagagactccggagcctggcaggcagacctgttctgacaccgaacttccaaagtcatgggccttgattggggtggtctgaattagacctagcccttttctgggcagaagggagcttctaggaggatggatgctgttcgggttagagctcgtgtggacctagctgcaggcaaaagccttgaggctgagtcccttctgtggcatggtggacagactctcgctcatcacagccgggcttgtcacgggagctcctcctccacacccctccctaagctgcctgtatggacgcggccctctgacactgaggtcggagttatcatttcaaaaccttgctctgtattaaacagccgtgttgggcagggccagactgctggactgacagtagggggcaggcagccggaccctctgagctccccaacggcaccagcgcctgcacggcctcagcccagggggtcattagggaagctctccccgattctgtgcagacagagcttcctctgtccacccttgctcggccagaattgtgtgccgctggtgactggcacccctctattctagggccaaggcctctcaggggtctacagatacaactatgggtgggtgcacacccatgttataaaccacactaaatgcacaaaaactgtctgcaagtttggggtgcggggaacagctctgggtgggaggttggaaatttggtctgggggacccactcggctccctccctcagcccacagtgagtctggtttctgagttgtcccggtctagccactttcgtttcccctggggccgggtggaggctggggcagaaagcggaactgagcccgcgtgttctgaggccagggcagggctggagcgttctgaacacctccccgtcccagcccctgggccaggcaagggccggccttacctctcctgggttggtggcagcagagctgggctctgagggaggcctgcaatgtgagacagtagcagctcagaggcggcactaggcaggtgcaaccccaaaaggtactgggcagggaatttcaggggaaactgaggctccatgctgcaaggccaaagcaggcccagacacagggagttcagctcgaacgtttgctccttacttgctgaccgacacattcacttctgatgggctgtgctgtgtaatgacccagtcctttctgtcttccacccaggggtctcaagtgaagggccaagggcatgggtaaggggaggagagggacagaaggggactcaggactgtggagagtttccggttctgggctggagagggagttgggcaggtgaaggggaggaggcagggtccctctggcctgactccagggaggcagtgaagctgtggcctgggaggcggggctcgtgcccctgggtaggtagcagttagagctgtggcttctgtttcctgtagccttcctaacaggctccggcaggcgttagggccttcctaagtgctacccgaatgcgtgtccagtgggaggagaagggggaggaccaagaacctagatgaatggccctagagaggtaacatctgtttggtgcctgctgtcattggaggtgggttgagttggttgatgggatttctctaaagatattgagaaactgctttcctctgagggcaggtgtccttgggagctccagatgagattcttgttgagggcatctccggagggaactctcaggggatggagggtgagaaaacttgggcagggaagaatcgaactaggggtgtagactgagcaggaggctgtcttcgggctgatcccacagagcgctcgctgtcccaggtagccagcttgagacaagacagccgggcttttggtgtcaggcagtcactggctgtgggctgccccccaagagcaaggggtgggcctaacctgtggtgtgagtagtggaaggcggttctctggccaacgggctgcttgctgctgttagcagttggagaatggatgcctctgcccggtaaagggcacctggggccgcgcccgcagcattcactaactcgtaactctccttcccttcctccaaacacaaaattccatgacactagacaagaaagctgatgcttggaaaagagaattggccttaaatacctagatggactggagagaccatcctttgtggtccatagcttgacctctgagtaccctgtccccatccacctgcagacctgctgaggctcccctctggctttctcctgcagacccctctgccatgaaccagtccatcccagtggctcccaccccaccccgccgcgtgcggctgaagccctggctggtggcccaggtgaacagctgccagtacccagggcttcaatgggtcaacggggaaaagaaattattctgcatcccctggaggcatgccacaaggcatggtcccagccaggacggagataacaccatcttcaaggtaagccccggggaggaggttggctggacctccagggcaccctgtccccagaagaggagcgcacataacgcacacaggcagctcctcgaggctggccacccgcccagctaccatgctgctgctgatgccgggcccggactaaggggatgcagacgtagacacagggtacacctttttcctttttttttttttttaagacagagtctcgctctgtagcccaggctggagtgcagtggcacgatctcagctcactgcaacctctgcctcctgggttcaagcaattctcctgcctcagcctcctgagtagctgggattacaggcatgagccaccacgcctggcctagggcacatcttttctaacctgcaccctagagcatcgtggggactgagggtccccagaaggccttcccataactcgtcctactcaccctttgctcgtctcactcctattactcatgaggacttgttcagtgcacgcatatgctaaaggaagccaacgatcatcatctttctaaaaattttatttttaaattagtatatatttatggggtacacagtggtgatttgataagcacagtgatcagatcaggtacttagcatatccataatctcaaacatttgtcatttctttgtgttgggaacaaactatttttaatataagattcagatacatcatcaatctttcaattgtttaaattttctaattttttttagagacagggcctcacactgttgaccagattccaattttttttttttttttttttttttttttgagacagggtctcactctgtcacccaggctggagtgcagtggcttgatctcagctcactgcagcctcgacctcccaggctcaggtgagtctcccatctcaacctccttgagtagctgggattacaggtgcctgccaccacaactggctaattttttgtacttttagtagagactttgccatgttgcccaggctggtcttgaactcctggactcaagcaatccacccacctcagcctcccagagtgctgggattacaggtgtgagccaccatgcccggcccaatctttttttttaattgatgtactacaactgtacatatatactctttttttttttttttgagatgagttgttgctctgttgcctagtgttgcaccattggtgagcagtggtgcagtcatagcacaacctccaactcagctcaagtgatcctccagtctcagcttcctgagtagccgtgactacaggtgcatgccaccatgcccagctaatttttttttttttttttgagatggagtcttgctttttcaccctagctggagtgcaatggcacaatctcagctcactgcaacctctgccacctgggttcaagcaattctcctgcctcagcctcccgagtagctaggattacaggcacctgccaccatgcccggctaatttttttttttttttttttttttttttttttttttgtatttttggtaaagacagggtttcaccctgttggccaggctggtcttgaactcctgacctcatgatccacctgccttggcctcccaaagtgctgggattacaggcgtgagccaccgtgcccagcctaattttttgattttgatatttgtaaagatgaggtctcactttgttgcccaggctggtctcaagctcatgggctcaagtgatcctcccacctcagcctcctgagtagctggtatcacaggcgcaagccactgtgctctctccaataatcttaatgctaggatgtacctcgcataatagtttttgcttacattttagtttttttgcacatgtgtgtatatggaatgcaaaattggaatcaagtgaatatcttgatttatagcctggatttttttcacccaatatcaggatcagctttcctttccaacatacgtccacatcacttttcgtgactgtagaatttccactggaatggtttagcacaatttggtcactgagtcttggtttttagatacttgagattgcttttaaaacctgaacttaaaaaccacattgtcatgtaggctgtcttaatgcttccctttttttcatcctcaattattttgggggttgatttcctagagttgaaaattgctgggtcaaaggcaatgcttattttttaaaactttgggtacatattttcttagtttttgggcttgatccttacctttccaaaattcttttgctaatccatttattttgtataggtaatacagtcacaaaattcaacattaaaaggcatagaatggtttagagcaaaaagtctccctccttccctggttcatcaccacccactcccctccctagcctttccctagaggcagccgtgttgtcattttcttgtttagccttcctgagagatttttatgcacatgtaagcaaatatggaactatcctttcctctacttttcataagaaatgctagatatctgcatatttgtctagacttaatacttcttgacgattgctacatgtcagcttataaacagtttcctgctttttctttttttgtgctgcccagtatttttcattcaatgggttggccgtaatttcaccagcccctcattgatggatgttgcattgggtattttgggtcatcttttacacacagcactgctgcgaataactttgtgtgtgcaatattttgtgtgaatgcttctatgatatttaggatgaattcctagaaatcaaatagctgggttaaaaggtagaaagaaatgttggtaaccctcacctcacctaattgccattcaaaataataccaacagctccagtgtctcgagcctgtcctatgtgcaggtttagctctgagtgctttacggacatcaactcccctcatcttttttgagatagggtatcactctgtcgtccaggctggagtgcagagcataatcatggctcactgcagccttgaactccccagctcaagcaatcctctcacctcagcctcctgagtagctgggaccacaggcacccaccactatgcccagctaattttttgtaaagacaggagtctcgttatgttgcctaggctggactccaactcctgggctcaagcagtcctcccaccttggcctcccaaaatgttgggattacaggtgcgagccactgtgcccagccaacatcaaccccttgagtcttcgctgccactccatgaggtgggcaactgtgaggagatcaagacaaagggaggcagtgacatggcctggcacacagctggcggggggcacttccagattcaaacccagcctggctccagggtgctcacccttatcccgcacgctgtcctccttagagatcacactgtttcaccctcccaggagcagggactatggatgcatctcatagcccctagatttcatgtagcttgctggcagcctgagggctggggcaggactgtggcagactcccacgctgcaaaccaggtctggggctcagcgaggctcagcctgtagccgaagttctccccacacagtagagtctcctatgggacaggaggcagactggggctgtggcagggatgaggttctctgtggtcggctatttcttcctgccccaggcctgggccaaggagacagggaaatacaccgaaggcgtggatgaagccgatccggccaagtggaaggccaacctgcgctgtgcccttaacaagagccgggacttccgcctcatctacgacgggccccgggacatgccacctcagccctacaagatctacgaggtctgctccaatggccctgctcccacaggtatcaggcctagccctctgtgggccacctgggaggctgtgcaatgtcctggcccccagccatgagctcttgggtgcaggcaggccaagggcccctctagcaggcagtggtccaggaaacgatgcgggggctcccgctaggtcatgacacccagggcttccaggagtggctgggatgggtcactggcatatcaggaatggcttggcgtgcagtcagggacctgggtgcttcttccttaccattgccctcgttttggcttctggctccagcctaggtctcatggcccatggagtcggggaggtctttcccaatcctggtggctgtgccctccacctcgccctgtgttgggggcagctttggggaaggcagaagctgcataggagctacaggcagcctctcaggggatcttgcttctcctccgacattgactcctttactgccctgcttttctctccctgctgtgcagactcccagccccctgaggattactcttttggtgcaggagaggaggaggaagaagaggaagaggtgagtgtgggttgaggaggcaggtggagccctggacgagctctctgctgtccccatcggccttaggtttccgcagccccactcccatggagccccgtggccctctcaatagttctccttgtttcttctcctgggattctgaacgataggagcacagtccccacctgctccttcccagggcattgtcattaccctgtgtgtgtgacccacgcagcagttggggcttggtaggtctgactccctgcagaaggcaaatgaggaaagtgaggcaaagggcttttctgacctgcctgggatggacgagctgggaccggaggcagggtcttgcctgagctaaactgaggctaggggagttgcctcatagttctcgcctgttatttccccagccccaggtcagtggaataacctgtcctcctttctctcccatctcttccctcccttgctggtggtgtcccttcagctgcagaggatgttgccaagcctgagcctcacaggtggggccgggaggtggtggttgggggtctagtatacagagaagctataggtaccataggtacctggaagggggctgatgggaggctagggtggcccagggctgggaggaggtgtgcctgggaggcagttcgtggaggtggcactgacagccgtccacacgcactctctgtagatgcagtgcagtctggcccccacatgacaccctattctttactcaaagaggatgtcaagtggccgcccactctgcagccgcccactctgcggccgcctactctgcagccgcccactctgcagccgcccgtggtgctgggtccccctgctccagaccccagccccctggctcctccccctggcaaccctgctggcttcagggagcttctctctgaggtcctggagcctgggcccctgcctgccagcctgccccctgcaggcgaacagctcctgccagacctgctgatcagcccccacatgctgcctcgtaaggacccatggctgggcacggggaagcagtgctgggggattggggtaggattggcaaggagggtggagggtgctggactcccttgggtgggaaaagtgggagggcggatggggctgggcctggccactgggctgcagaatggggaggcgtggggctcaaggacgggatgggcctgccttctgccccacagtgaccgacctggagatcaagtttcagtaccgggggcggccaccccgggccctcaccatcagcaacccccatggctgccggctcttctacagccagctggaggccacccaggagcaggtggaactcttcggccccataagcctggagcaagtgcgcttccccagccctgaggacatccccagtgacaagcagcgcttctacacgaaccagctgctggatgtcctggaccgcgggctcatcctccagctacagggccaggacctttatgccatccgcctgtgtcagtgcaaggtgttctggagcgggccttgtgcctcagcccatgactcatgccccaaccccatccagcgggaggtcaagaccaagcttttcagcctggagcattttctcaatggtgagggcccaaagctgtgatcctcctggctgcctcttgcccagggcatggttccagcctctgactagggaccttgattttgatgcagagctcatcctgttccaaaagggccagaccaacaccccaccacccttcgagatcttcttctgctttggggaagaatggcctgaccgcaaaccccgagagaagaagctcattactgtacaggtacatctcccctatcccaaagtcggccttggcttgaaaactggggaatcctggggctaggcccttgccccaggctggaggctcagggctccctgagcagtgtgaacttggcggccagagaccatcaaggctcagagccggagaatgcggtctattactcacccctgatggctgtcctcatgcacagctggatctggcagccctgccacaggtctccctgtctcatctcctctttgcctcccaggtggtgcctgtagcagctcgactgctgctggagatgttctcaggggagctatcttggtcagctgatagtatccggctacagatctcaaacccagacctcaaagaccgcatggtggagcaattcaaggagctccatcacatctggcagtcccagcagcggttgcagcctgtggcccaggcccctcctggagcaggccttggtgttggccaggggccctggcctatgcacccagctggcatgcaataacaaggctgcagacggtgactggccctggcttcctgggtggcggtgcggactgatgtggagatgtgacagccccgatgagcacctggctggctgcagggtcctacctctgggtttcctggaagtggatttgggccaagaaggagagggagaaaggcccgagcccctgccttcccgggcctttctctcctgggctgtctctggtctggtcagcctggctctcgggaaattcagccatgagcagggaaagaactctcccaaccctggggcctagctgtataggaggaattgcctaagggtggcccactcttgtgattgccccatttcctctggcaacaaaagccagagtgttgtgggccaagtccccccacagggcctctgcagggcatggccctgatttccctggtttgagactcacttcctcatctccctgtcctctgagataatatgagtgagcacttaggtatcatatcagatgctcaaggctggcagctacccccttcttgagagtccaagaacctggagcagaaataatttttatgtatttttggattaatgaatgttaaaaacagactcagctgtttctttccttttactactaccagttgctcccatgctgctccaccaggccctgtttcggatgccaactggcccactccccaagcacttgcccccagcttgcgaccattggcactgggagggcctggcttctgggctgatgggtcagttgggccttcataaacactcacctggctggctttgccttccaggaggaagctggctgaagcaagggtgtggaattttaaatgtgtgcacagtctggaaaactgtcagaatcagttttcccataaaagggtgggctagcattgcagctgcatttgggaccattcaaatctgtcactctcttgtgtatattcctgtgctattaaatatatcagggcagtgcatgtaaatcatcctgatatatttaatatatttattatattgtcccccgaggtggggacagtgagtgagttctcttagtccccccagagctggttgttaaagagcctggcacctacccgctctcacttcatctgtgtcatctctgcacactccagcccactttctgccttcagccattgagtggaagctgccccaggcccttaccaggtgcagatgcccaatcttgatgcccagccatcagaactgtgagccaaataaacctttttctgtataaattacccagcctcgggtcttcgtttacagcaacgcaaaatagattaaacccccataaatgttcaaggataccttgccccacagcctcgtccacagaatatattgtcactgtttggatttttgccaacctgacaggtgagatagtatctcagtgccacttctcattatcagcaaggctgagtagcttttcacatggttaagtggcctgtacagatttttttaaataattttagaatggttttagatttatggaaaagttcctaatagagttcctatggacccacactttctccaattgttaacatcttacattactatggcacacttgtgacaataatgaaaccatgtggacaattactatgaactcaacttctttatttggatttcatgagtttttcggatatcctttttctgttccaggatactatccaagatatcacattacattcacttgtcacatctccttagactcctctggtctatgacagtttcttagacttttcttgttgttggtcttgatggttttgaggaatactggccattttttaaagcatgtcccccagtttgggcttatctgatgtatttctcatttgtttttagagatagggtctttgtgttgcccaggctggagtgccatggtgcaaccttgaactcctgggctcagatgatcctcccacgtagctgggactacgggtgcacaccaccacacctggctttttttttttgagacctggtctcaatatattgcccaggctggtctcaaactcctggcctcaagtgatcttcccaccgtggcctcccaaacagctgagattacaggtgtgaaccactgtgccccgcctgtttctcatggtttttgtatggaagaccacagaggtgaagcatcattcccactacatcatatcaagggtgcatactattaacctgatttatcactgtttctgactgaggtagtgataggttcctctatcatagttactcttcatcctccctgatctatatgtacacaggtaccgtggttttagtattattcacctgcatcctcttaaacttcaccaactagagtacagtgattacgtagtgctccttttgtcttagttttcaatcatttacaaaggtgtgcacctttttcttcacaggcctgtgcagttctactccctcttgcaattgtcaatgttttgagattaggagaagggagatgtcacatatctggtttccagcttcttccaaaaaactggcaaatatgaaaacactgtcatacattcctcaaaggcaaaaatcagctggaactctggatggggaatgttctcccagttcaatacggtcctctcctggcctgcttcctttcactgctctccgtgtccctaagggcatttgagtttgcaacccttgatgaatggtggagcagggagactgagtgtagatgaattggggaagcttggaagggccaggtgcccctcgcgaccacgagtgggctgacttccccagtactgctagcagttgagtaactgccaaaaatcttccctgaatttgtattaattggtttcctaaaactaaaatgaaaacagcaatgaaaacctggctgtcacttacagatctctgtgttgtagtagccagctgtggatgaacagtatccagaagtgacagatgtaacaaactccagaggtaaaggcaggccagtgtgaggtacaattcactgctcccaagccaggctagggctccccttggtaatagcttttggctgctctggtggcctgtgctggagcttaagttggaacccacaggaccttgatgcccacatcctggggatgaatgcatccctttagccccagcagtttctctcccttctttcaagctccttgtccccaccaagttcatccttataaccaactaaatataccctttttaagactctcagctctttgacataaagaacctaccctggccgggtgcagtggctcacgcctgtagtcccagcactttgggaggccgaggtgggcagatcacgaggtcaggagttcgagaccagcctggccaacatggtgaaaccccatctctactaaaaatacaaatattagctggtcatggtggtgggtgcctgtaatcctagctgctcaggaggctgaggcaggagaatcgctggaccccatcagacagaggttgcagtgagctgaaatcccaccattgcactccagcctgggcaacagagcaaggctccctctcaaaaaaaaaaaaacaaaaaaacaaaacaaaacaaaacaaaacaaaaggaaaaaaagaaaaaacgtacccttttcagtctctgtagagtagaagatatcaatggcttttcccatttttgctgaaagagcattcttggaaggcaatgttctcatttgctggtaatctatcccaaagaatcaggcacatcttgttgcaaaatcttgaacctttaaggtctcttggttttggctttcatcttatgattcacttaaatcagggccacacaaggtagacatttaaataaactcatgaatgttgatgccattcaaaagcacagtttcctttatgtcttaggtgtgcattctcagttactgttctttataacaattcccttcagatacataaggattacaagttactgtgttttagtacagtcttgctggaaatagccccaatgctgatgatctgtgttccacataatcatggccaacatttcatggctaatcctatcacggaacagacaattctgtggggagatgacttaggcaagttcaggtgcagttagctgacctgcccacatggacgggagatactcatcctatttctactagacaatgccatgcagaaacacagcatctccattaggtgagctgatcctgaacagttcctttttaaattaaaatctagatgggcatccttgctctgccgaaagtttcagagcagccctttcaaataatctgtaaggcttaagtaagcagtgaggtt

SEQ ID NO: 10 - DNA, 16 bp, Artificial Sequence
  Description of Artificial Sequence Synthetic oligonucleotide
cctatacagctaggcc

SEQ ID NO: 11 - DNA, 16 bp, Artificial Sequence
  Description of Artificial Sequence Synthetic oligonucleotide
tctgatatgataccta

SEQ ID NO: 12 - DNA, 16 bp, Artificial Sequence
  Description of Artificial Sequence Synthetic oligonucleotide
tatttctgctccaggt

SEQ ID NO: 13 - DNA, 16 bp, Artificial Sequence
  Description of Artificial Sequence Synthetic oligonucleotide
ctgatatgatacctaa

SEQ ID NO: 14 - DNA, 16 bp, Artificial Sequence
  Description of Artificial Sequence Synthetic oligonucleotide
acgagttatgggaagg

SEQ ID NO: 15 - DNA, 16 bp, Artificial Sequence
  Description of Artificial Sequence Synthetic oligonucleotide
atgagtaataggagtg

SEQ ID NO: 16 - DNA, 16 bp, Artificial Sequence
  Description of Artificial Sequence Synthetic oligonucleotide
tgtctagtgtcatgga